(19) United States
(12) Patent Application Publication
(10) Pub. No.: US 2021/0261962 A1
(43) Pub. Date: Aug. 26, 2021

(54) CORRECTION OF DYSTROPHIN EXON 43, EXON 45, OR EXON 52 DELETIONS IN DUCHENNE MUSCULAR DYSTROPHY

(71) Applicant: The Board of Regents of the University of Texas System, Austin, TX (US)

(72) Inventors: Yi-Li MIN, Dallas, TX (US); Rhonda BASSEL-DUBY, Dallas, TX (US); Eric OLSON, Dallas, TX (US)

(73) Assignee: The Board of Regents of the University of Texas System, Austin, TX (US)

(21) Appl. No.: 17/253,606

(22) PCT Filed: Jun. 21, 2019
(86) PCT No.: PCT/US2019/038398

§ 371 Date: Dec. 17, 2020

Related U.S. Application Data
(60) Provisional application No. 62/854,131, filed on May 29, 2019.
(60) Provisional application No. 62/688,003, filed on Jun. 21, 2018.

(51) Int. Cl. C12N 15/113; C12N 9/22; C12N 15/86; A01K 29/00

(52) U.S. Cl. CPC C12N 15/113; C12N 9/22; C12N 15/86; A01K 29/005; C12N 2750/14143; A01K 2207/05; C12N 2320/33; A01K 2267/0306; A01K 2227/105; A01K 2217/206; C12N 2310/20; C12N 2830/50

(57) ABSTRACT

Duchenne muscular dystrophy (DMD), which affects 1 in 5,000 male births, is one of the most common genetic disorders of children. This disease is caused by an absence or deficiency of dystrophin protein in striated muscle. The major DMD deletion “hot spots” are found between exon 6 to 8, and exons 45 to 53. Here, three DMD mouse models are provided that can be used to test a variety of DMD exon skipping and refraining strategies. Among these are, CRISPR/Cas9 oligonucleotides, small molecules or other therapeutic modalities that promote exon skipping or exon refraining or micro dystrophin mini genes or cell based therapies. Methods for restoring the reading frame of exon 43, exon 45, and exon 52 deletion via CRISPR-mediated exon skipping and refraining in the humanized DMD mouse model, in patient-derived iPSCs and ultimately, in patients using various delivery systems are also contemplated. The impact of CRISPR technology on DMD is that gene editing can permanently correct mutations.

### CROSS-REFERENCE TO RELATED APPLICATIONS

This application claims priority to U.S. Provisional Application Ser. No. 62/854,131, filed May 29, 2019, and U.S. Provisional Application Ser. No. 62/688,003, filed Jun. 21, 2018, each of which is incorporated by reference herein in its entirety for all purposes.

### FEDERAL FUNDING SUPPORT CLAUSE

This invention was made with government support under grant no. U54 HD 087351 awarded by National Institutes of Health. The government has certain rights in the invention.

### SEQUENCE LISTING

The instant application contains a Sequence Listing which has been submitted electronically is hereby incorporated by reference in its entirety. The sequence listing was created on Jun. 20, 2019, is named UTFD_P3391WO.txt and is ˜6,540,494 bytes in size.

### FIELD

The present disclosure relates to the fields of molecular biology, medicine and genetics. More particularly, the disclosure relates to the use of genome editing to create humanized animal models for different forms of Duchenne muscular dystrophy (DMD), each containing distinct DMD mutations.

### BACKGROUND

Muscular dystrophies (MD) are a group of more than 30 genetic diseases characterized by progressive weakness and degeneration of the skeletal muscles that control movement. Duchenne muscular dystrophy (DMD) is one of the most severe forms of MD that affects approximately 1 in 5000 boys and is characterized by progressive muscle weakness and premature death. Cardiomyopathy and heart failure are common, incurable and lethal features of DMD. The disease is caused by mutations in the gene encoding dystrophin (DMD), a large intracellular protein that links the dystroglycan complex at the cell surface with the underlying cytoskeleton, thereby maintaining integrity of the muscle cell membrane during contraction. Mutations in the dystrophin gene result in loss of expression of dystrophin causing muscle membrane fragility and progressive muscle wasting.

There remains a need in the art for treatments and cures for DMD, and for mouse models to be used for developing the same.

### SUMMARY

In accordance with the present disclosure, there is provided a nucleic acid comprising a sequence encoding a single guide RNA (sgRNA) comprising a spacer sequence and a scaffold sequence; wherein the spacer sequence comprises the sequence of any one of SEQ ID NOs: 135-146, 170-260, 337-339, or 617. The scaffold sequence may comprise, for example, the sequence of any one of SEQ ID NO: 147-153. In some embodiments, the nucleic acid comprises one copy of the sequence encoding the sgRNA. In some embodiments, the nucleic acid comprises two, three, four, or five copies of the sequence encoding the sgRNA. The nucleic acid may comprise a sequence encoding a promoter, wherein the promoter drives expression of the sgRNA. In some embodiments, the nucleic acid comprises three copies of the sequence encoding the sgRNA, wherein the nucleic acid comprises a sequence encoding a first promoter and expression of the first copy of the sgRNA is driven by the first promoter, wherein the nucleic acid comprises a sequence encoding a second promoter and expression of the second copy of the sgRNA is driven by the second promoter, and wherein the nucleic acid comprises a sequence encoding a third promoter and expression of the third copy of the sgRNA is driven by the third promoter. In some embodiments, the nucleic acid further comprises a sequence encoding a nuclease. In some embodiments, the nuclease is a Type II, Type V-A, Type V-B, Type V-C, Type V-U, or Type VI-B nuclease. In some embodiments, the nuclease is a TAL nuclease, a meganuclease, or a zinc-finger nuclease. In some embodiments, the nuclease is a Cas9, Cas12a, Cas12b, Cas12c, Tnp-B like, Cas13a (C2c2), Cas13b, or Cas14 nuclease. The Cas9 nuclease may be, for example, a Streptococcus pyogenes or Streptococcus aureus Cas9. In some embodiments, the Cas9 nuclease is a modified Cas9 nuclease, such as a modified Streptococcus pyogenes Cas9 or a modified Streptococcus aureus Cas9.

Also provided is a recombinant vector comprising a nucleic acid of the disclosure. In some embodiments, the recombinant vector is an expression vector. In some embodiments, the recombinant vector is a viral vector. In some embodiments, the viral vector is a lentiviral vector, a retroviral vector, an adenoviral vector, or an adeno-associated virus (AAV) vector. In some embodiments, the viral vector is an adeno-associated virus (AAV) vector. The AAV vector may have a serotype of, for example, AAV1, AAV2, AAV3, AAV4, AAV5, AAV6, AAV7, AAV8, AAV9, AAV10, AAV11, AAV12, AAVRh74, AAV2i8, AAVRh10, AAV39, AAV43, AAVRh8, avian AAV, bovine AAV, canine AAV, equine AAV, or ovine AAV. In some embodiments, the serotype of the AAV vector is AAV9. In some embodiments, the AAV vector is replication-defective or conditionally replication defective.

Also provided is a non-viral vector comprising a nucleic acid of the disclosure. The non-viral vector may comprise calcium phosphate, liposomes, nanoparticles, and/or lipid emulsions.

Also provided is an AAV expression cassette comprising a first inverted terminal repeat (ITR); a first promoter; a nucleic acid of the disclosure; and a second ITR. In some embodiments, the AAV expression cassette further comprises a polyadenosine (polyA) sequence. In some embodiments, one or both of the first ITR and the second ITR are isolated or derived from any one of AAV1, AAV2, AAV3, AAV4, AAV5, AAV6, AAV7, AAV8, AAV9, AAV10, AAV11, AAV12, AAVRh74, AAV2i8, AAVRh10, AAV39, AAV43, AAVRh8, avian AAV, bovine AAV, canine AAV, equine AAV, or ovine AAV.

Also provided is an AAV vector comprising a nucleic acid or an AAV expression cassette of the disclosure. In some embodiments, the AAV vector has the serotype of AAV1, AAV2, AAV3, AAV4, AAV5, AAV6, AAV7, AAV8, AAV9, AAV10, AAV11, AAV12, AAVRh74, AAV2i8, AAVRh10, AAV39, AAV43, AAVRh8, avian AAV, bovine AAV, canine AAV, equine AAV, or ovine AAV. In some embodiments, the serotype of the AAV vector is AAV9. In some embodiments, the AAV vector is replication-defective or conditionally replication defective.

Also provided is a composition comprising a nucleic acid, an AAV expression cassette, or an AAV vector of the disclosure. In some embodiments, the composition further comprises a pharmaceutically acceptable carrier.

Also provided is a cell comprising a nucleic acid, an expression cassette, an AAV vector, or a composition of the disclosure. In some embodiments, the cell is a stem cell. In some embodiments, the cell is a mammalian cell such as a human cell.

Also provided is a method of correcting a gene defect in a cell, the method comprising contacting the cell with a nucleic acid, a recombinant vector, a non-viral vector, an AAV vector, or a composition of the disclosure. In some embodiments, the cell is a stem cell. In some embodiments, the cell is a mammalian cell such as a human cell.

Also provided is a method of treating a subject suffering from Duchenne muscular dystrophy, the method comprising administering to the subject a therapeutically effective amount of a nucleic acid, a recombinant vector, a non-viral vector, an AAV vector, or a composition of the disclosure.

Also provided is a method of treating a subject suffering from Duchenne muscular dystrophy, the method comprising administering to the subject a first vector (e.g., a recombinant vector or non-viral vector of the disclosure), and a second vector, wherein the second vector encodes a nuclease. In some embodiments, the nuclease is a Type II, Type V-A, Type V-B, Type V-C, Type V-U, or Type VI-B nuclease. In some embodiments, the nuclease is a TAL nuclease, a meganuclease, or a zinc-finger nuclease. In some embodiments, the nuclease is a Cas9, Cas12a, Cas12b, Cas12c, Tnp-B like, Cas13a (C2c2), Cas13b, or Cas14 nuclease. The Cas9 nuclease may be, for example, a Streptococcus pyogenes or Streptococcus aureus Cas9. In some embodiments, the Cas9 is a modified Cas9 nuclease, such as a modified Streptococcus pyogenes Cas9 or a modified Streptococcus aureus Cas9. In some embodiments, the second vector is a plasmid. In some embodiments, the second vector is an expression vector. In some embodiments, the second vector is a viral vector. In embodiments wherein the second vector is a viral vector, it may be a lentiviral vector, a retroviral vector, an adenoviral vector, or an adeno-associated virus (AAV) vector. In some embodiments, the viral vector is an adeno-associated virus (AAV) vector. The serotype of the AAV vector may be selected from AAV1, AAV2, AAV3, AAV4, AAV5, AAV6, AAV7, AAV8, AAV9, AAV10, AAV11, AAV12, AAVRh74, AAV2i8, AAVRh10, AAV39, AAV43, AAVRh8, avian AAV, bovine AAV, canine AAV, equine AAV, and ovine AAV. In some embodiments, the second vector is a non-viral vector, wherein the non-viral vector comprises calcium phosphate, liposomes, nanoparticles, and/or lipid emulsions. In some embodiments, the administering induces a frameshift mutation in a target nucleic acid sequence in a cell of the patient. In some embodiments, the frameshift mutation comprises a deletion of at least one nucleotide, wherein the number of nucleotides deleted is not a multiple of 3 (e.g., a deletion of 1, 2, 4, 5, 7, 8, 10, 11, 13, 14, 16, 17, 19 or 20 nucleotides.) In some embodiments, the frameshift mutation comprises an insertion of at least one nucleotide, wherein the number of nucleotides inserted is not a multiple of 3 (e.g., an insertion of 1, 2, 4, 5, 7, 8, 10, 11, 13, 14, 16, 17, 19 or 20 nucleotides.) In some embodiments, the frameshift mutation comprises an insertion of 1 nucleotide. The first vector and the second vector may be administered simultaneously, or may be administered sequentially. In some embodiments, the first vector and the second vector may be administered locally (e.g., to a muscle tissue), or may be administered systemically. In some embodiments, the first vector and the second vector are administered by an oral, rectal, transmucosal, topical, transdermal, inhalation, intravenous, subcutaneous, intradermal, intramuscular, intra-articular, intrathecal, intraventricular, intravenous, intraperitoneal, intranasal, or intraocular route of administration. The subject suffering from DMD may be greater than or equal to 18 years old, less than 18 years old, or less than 2 years old. In some embodiments, the subject is a human. In some embodiments, the ratio of the first vector to the second vector is 1:1 to 1:100. In some embodiments, the ratio of the second vector to the first vector is 1:1 to 1:100.

Also provided is a combination therapy comprising a first composition comprising a first vector comprising a nucleic acid of the disclosure, and a second composition comprising a second vector comprising a nucleic acid that encodes a nuclease. The first and/or the second composition may comprise a pharmaceutically acceptable carrier. In some embodiments, the nuclease is a Type II, Type V-A, Type V-B, Type V-C, Type V-U, or Type VI-B nuclease. In some embodiments, the nuclease is a TAL nuclease, a meganuclease, or a zinc-finger nuclease. In some embodiments, the nuclease is a Cas9, Cas12a, Cas12b, Cas12c, Tnp-B like, Cas13a (C2c2), Cas13b, or Cas14 nuclease. In embodiments wherein the nuclease is a Cas9 nuclease it may be, for example, a Streptococcus pyogenes or Streptococcus aureus Cas9. In some embodiments, the nuclease is a modified Cas9 nuclease, such as a modified Streptococcus pyogenes Cas9 or a modified Streptococcus aureus Cas9.

Also provided is a composition, a recombinant vector, or a non-viral vector of the disclosure for use as a medicament.

Also provided is a composition, a recombinant vector, or a non-viral vector of the disclosure for use in the treatment of Duchenne muscular dystrophy.

Also provided are three different mouse models, wherein the genome of each mouse model comprises a deletion of exon 43, exon 45, or exon 52 of the dystrophin gene, resulting in an out of frame shift and a premature stop codon in exon 44, exon 46, and exon 53, respectively. These mutations are similar to mutations found in approximately 18% of human DMD patients, and correction of these deletions through exon skipping or reframing of surrounding exons can be used to treat DMD. The genome of these mice may further comprise a reporter gene located downstream of and in frame with exon 79 of the dystrophin gene, and upstream of a dystrophin 3′-UTR, wherein said reporter gene is expressed when exon 79 is translated in frame with exon 45, exon 47, or exon 54. The reporter gene may be luciferase. The genome of the mouse may further comprise a protease coding sequence upstream of and in frame with said reporter gene, and downstream of and in frame with exon 79. The protease may be autocatalytic, such as 2A protease. The mouse may be heterozygous for said deletion, or homozygous for said deletion. The mouse may exhibit increased creatine kinase levels, and/or may not exhibit detectable dystrophin protein in heart or skeletal muscle.

Also provided is a method of producing the mouse described above comprising (a) contacting a fertilized oocyte with CRISPR/Cas9 elements and two single guide RNA (sgRNA) targeting sequences flanking exon 43, exon 45, or exon 52, thereby creating a modified oocyte, wherein deletion of exon 43, exon 45, or exon 52 by CRISPR/Cas9 results in an out of frame shift and a premature stop codon in exon 44, exon 46, or exon 53; (b) transferring said modified oocyte into a recipient female. The oocyte genome may comprise a dystrophin gene having a reporter gene located downstream of and in frame with exon 79 of said dystrophin gene, and upstream of a dystrophin 3′-UTR, wherein said reporter gene is expressed when exon 79 is translated in frame with exon 45, exon 47, or exon 54. The reporter gene may be luciferase. The oocyte genome may further comprise a protease coding sequence upstream of and in frame with said reporter gene, and downstream of and in frame with exon 79. The protease may be autocatalytic, such as 2A protease. The mouse may be heterozygous for said deletion, or homozygous for said deletion. The mouse may exhibit increased creatine kinase levels and/or may not exhibit detectable dystrophin protein in heart or skeletal muscle.

In another embodiment, there is provided an isolated cell obtained from the mouse described above. The genome of the cell may further comprise a reporter gene located downstream of and in frame with exon 79 of the dystrophin gene, and upstream of a dystrophin 3′-UTR, wherein said reporter gene is expressed when exon 79 is translated in frame with exon 45, exon 47, or exon 54. The reporter gene may be luciferase. The genome of the cell may further comprise a protease coding sequence upstream of and in frame with said reporter gene, and downstream of and in frame with exon 79. The protease may be autocatalytic, such as 2A protease. The cell may be heterozygous for said deletion, or homozygous for said deletion.

In a further embodiment, there is provided a mouse produced by a method comprising the steps of (a) contacting a fertilized oocyte with CRISPR/Cas9 elements and two single guide RNA (sgRNA) targeting sequences flanking exon 43, exon 45, or exon 52, thereby creating a modified oocyte, wherein deletion of exon 43, exon 45, or exon 52 by CRISPR/Ca9 results in an out of frame shift and a premature stop codon in exon 44, exon 46, or exon 53; (b) transferring said modified oocyte into a recipient female.

These mice provide an important system for assessing the efficacy of a variety of therapeutic analogues for correction of DMD mutations. In one embodiment, CRISPR/Cas9 can be used to skip or reframe exon 44, exon 46 or exon 53, putting the dystrophin protein back in frame. The mice allow for rapid optimization of the method. In another embodiment, the mice can be used to test exon-skipping oligonucleotides or small molecules or other therapeutic modalities in a “humanized” system. In still a further embodiment, there is provided a method of screening a candidate substance for DMD exon-skipping activity comprising (a) treating a mouse (e.g., a mouse from one of the mouse models described herein) with a candidate substance; and (b) assessing in frame transcription and/or translation of exon 79, wherein the presence of in frame transcription and/or translation of exon 79 indicates said candidate substance exhibits exon-skipping activity.

A further embodiment comprises an isolated nucleic acid comprising a sequence of any one of SEQ ID NO: 1-72, 340-359, or 360-515. Also provided is a double-stranded nucleic acid formed by hybridization of SEQ ID NO: 1 and 2, SEQ ID NO: 3 and 4, SEQ ID NO: 5 and 6, SEQ ID NO: 7 and 8, SEQ ID NO: 9 and 10, SEQ ID NO: 11 and 12, SEQ ID NO: 13 and 14, SEQ ID NO: 15 and 16, SEQ ID NO: 17 and 18, SEQ ID NO: 19 and 20, SEQ ID NO: 21 and 22, SEQ ID NO: 23 and 24, SEQ ID NO: 25 and 26, SEQ ID NO: 27 and 28, SEQ ID NO: 29 and 30, SEQ ID NO: 31 and 32, SEQ ID NO: 33 and 34, SEQ ID NO: 35 and 36, SEQ ID NO: 37 and 38, SEQ ID NO: 39 and 40, SEQ ID NO: 41 and 42, SEQ ID NO: 43 and 44, SEQ ID NO: 45 and 46, SEQ ID NO: 47 and 48, SEQ ID NO: 49 and 50, SEQ ID NO: 51 and 52, SEQ ID NO: 53 and 54, SEQ ID NO: 55 and 56, SEQ ID NO: 57 and 58, SEQ ID NO: 59 and 60, SEQ ID NO: 61 and 62, SEQ ID NO: 63 and 64, SEQ ID NO: 65 and 66, SEQ ID NO: 67 and 68, SEQ ID NO: 69 and 70, and SEQ ID NO: 71 and 72, and an expression construct comprising a nucleic acid formed by hybridization of SEQ ID NO: 1 and 2, SEQ ID NO: 3 and 4, SEQ ID NO: 5 and 6, SEQ ID NO: 7 and 8, SEQ ID NO: 9 and 10, SEQ ID NO: 11 and 12, SEQ ID NO: 13 and 14, SEQ ID NO: 15 and 16, SEQ ID NO: 17 and 18, SEQ ID NO: 19 and 20, SEQ ID NO: 21 and 22, SEQ ID NO: 23 and 24, SEQ ID NO: 25 and 26, SEQ ID NO: 27 and 28, SEQ ID NO: 29 and 30, SEQ ID NO: 31 and 32, SEQ ID NO: 33 and 34, SEQ ID NO: 35 and 36, SEQ ID NO: 37 and 38, SEQ ID NO: 39 and 40, SEQ ID NO: 41 and 42, SEQ ID NO: 43 and 44, SEQ ID NO: 45 and 46, SEQ ID NO: 47 and 48, SEQ ID NO: 49 and 50, SEQ ID NO: 51 and 52, SEQ ID NO: 53 and 54, SEQ ID NO: 55 and 56, SEQ ID NO: 57 and 58, SEQ ID NO: 59 and 60, SEQ ID NO: 61 and 62, SEQ ID NO: 63 and 64, SEQ ID NO: 65 and 66, SEQ ID NO: 67 and 68, SEQ ID NO: 69 and 70, and SEQ ID NO: 71 and 72, such as a viral or non-viral vector. Additionally, a kit comprising one or more isolated nucleic acids comprising the sequence of any one of SEQ ID NO: 1-72, 340-359, or 360-515 is provided.

Still a further embodiment comprises a method of correcting a dystrophin gene defect in exon 44, exon 46, or exon 53 of the DMD gene in a subject comprising contacting a cell in said subject with Cpf1 or Cas9 and a DMD guide RNA as defined above, resulting in selective skipping of a mutant DMD exon. The cell may be a muscle cell, a satellite cell, or an induced pluripotent stem cell (iPSC) or iPSC-derived cardiomyocyte (iPSC-CM). Cpf1 and/or DMD guide RNA may be provided to said cell through expression from one or more expression vectors coding therefore, such as a viral vector (e.g., adeno-associated viral vector) or as a non-viral vector. Cpf1 or Cas9 may be provided to said cell as naked plasmid DNA or chemically-modified mRNA.

The method of may further comprise contacting said cell with a single-stranded DMD oligonucleotide to effect homology directed repair or nonhomologous end joining (NHEJ). Cpf1 or Cas9, DMD guide RNA and/or single-stranded DMD oligonucleotide, or expression vectors coding therefor, may be provided to said cell in one or more nanoparticles. Cpf1 or Cas9, DMD guide RNA and/or single-stranded DMD oligonucleotide may be delivered directly to a muscle tissue, such as tibialis anterior, quadricep, soleus, diaphragm or heart. Cpf1 or Cas9, DMD guide RNA and/or single-stranded DMD oligonucleotide may be delivered systemically.

The subject may exhibit normal dystrophin-positive myofibers and/or mosaic dystrophin-positive myofibers containing centralized nuclei. The subject may exhibit a decreased serum CK level as compared to a serum CK level prior to contacting. The subject may exhibit improved grip strength as compared to a serum CK level prior to contacting. The correction may be permanent skipping of said mutant DMD exon, or more than one mutant DMD exon. The Cpf1 or Cas9 and/or DMD guide RNA may be delivered to a human iPSC with an adeno-associated viral vector.

It is contemplated that any method or composition described herein can be implemented with respect to any other method or composition described herein.

Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIGS. 1A-1D. Generation of mice with DMD exon 43, exon 45, or exon 52 deletion. (FIG. 1A) Outline of the CRISPR/Cas9 strategy used for generation of the exon 43, exon 45, and exon 52 deleted mice. (FIG. 1B) Hematoxylin and eosin (H&E) immunostaining of TA, diaphragm and cardiac muscle in exon 43, exon 45, and exon 52 deleted mice. (FIG. 1C) Dystrophin staining of TA, diaphragm and cardiac muscle in exon 43, exon 45, and exon 52 deleted and wild type (WT) mice. Dystrophin stains in red. Nucleus marks by DAPI stains in blue. (FIG. 1D) Serum creatine kinase (CK), a marker of muscle dystrophy that reflects muscle damage and membrane leakage was measured in WT and Δ44, Δ43, Δ45, and Δ52 DMD mice.

FIGS. 2A-2C. Identification of optimal sgRNAs for CRISPR/Cas9 correction of DMD exon 43, exon 45, and exon 52 deletions. (FIG. 2A) Illustration of correction strategies for exon 43, exon 45, and exon 52 deletions. (FIG. 2B) T7E1 assay using 10T ½ mouse cells transfected with SpCas9 and exon 44, exon 46, or exon 53 targeting sgRNAs shows cleavage of the DMD locus. (FIG. 2C) T7E1 assay using 293 human cells transfected with SpCas9 and exon 44, exon 46, or exon 53 targeting sgRNAs shows cleavage of the DMD locus. gRNA spacer sequences used to perform these experiments are listed in Table 2 and Table 3, and the gRNA scaffold sequence used in combination with each spacer sequence corresponds to SEQ ID NO: 147.

FIGS. 3A-3C. DMD patient iPSC-derived cardiomyocytes express dystrophin after CRISPR/Cas9 mediated genome editing by exon skipping (FIG. 3A) Western blot analysis shows restoration of dystrophin expression in cardiomyocytes differentiated from exon 44-skipped iPSCs with exon 43 deletion. Vinculin is loading control. (FIG. 3B) Western blot analysis shows restoration of dystrophin expression in cardiomyocytes differentiated from exon 53-skipped iPSCs with exon 52 deletion. Vinculin is loading control. (FIG. 3C) Immunostaining shows restoration of dystrophin expression in exon 44-edited and exon 53-edited cells. Dystrophin stains in red. Cardiac troponin I stains in green. Nucleus marked by DAPI stains in blue. For ΔE43, the hDMD-E44g1 and hDMD-E44g4 spacers were used, and for ΔE52, the hDMD-E53g4 spacer was used. The gRNA scaffold sequence used in combination with each spacer sequence corresponds to SEQ ID NO: 147.

FIGS. 4A-4C. Identification of optimal sgRNAs for CRISPR/Cas9 correction of DMD exon 52 deletions. (FIG. 4A) Illustration of sgRNA selection strategies for targeting exon 53. (FIG. 4B) Location of sgRNAs targeting exon 53. (FIG. 4C) Western blot analysis shows restoration of dystrophin expression in cardiomyocytes differentiated from exon 53-skipped iPSCs with exon 52 deletion. Vinculin is loading control.

FIGS. 5A-5B. Identification of optimal sgRNAs for CRISPR/Cas9 correction of DMD exon 43 deletions. (FIG. 5A) Illustration of sgRNA selection strategies for targeting exon 44. (FIG. 5B) Location of sgRNAs targeting exon 44.

FIGS. 6A-6D. Identification of optimal sgRNAs for CRISPR/Cas9 correction of DMD exon 45 deletions. (FIG. 6A) Illustration of sgRNA selection strategies for targeting exon 44. (FIG. 6B) Location of sgRNAs targeting exon 44. (FIG. 6C) Western blot analysis shows restoration of dystrophin expression in cardiomyocytes differentiated from exon 44-skipped (SK) or reframed (RF) iPSCs with exon 45 deletion. The numbers #8, #44, #17, and #28 refer to single clones of the corrected iPSC line, with different indels. #8 and #44 were corrected by reframing (−1 nucleotide), and #17 and #28 were corrected by exon skipping. Vinculin is loading control. (FIG. 6D) Immunostaining shows restoration of dystrophin expression in exon 44-edited cells with exon 45 deletion. Dystrophin stains in red. Cardiac troponin I stains in green. Nucleus marked by DAPI stains in blue. For ΔE45, the hDMD-E44g4 spacer was used and the gRNA scaffold sequence corresponds to SEQ ID NO: 147.

FIGS. 7A-7B. Identification of optimal sgRNAs for targeting exon 46. (FIG. 7A) Illustration of sgRNA selection strategies for targeting exon 46. (FIG. 7B) Location of sgRNAs targeting exon 46.

FIGS. 8A-8B. Editing in DMD exons 44, 46, and 53. (FIG. 8A) Diagram of the exon editing strategy for DMD exon 43, exon 45 and exon 52 deletion (FIG. 8B) TIDE analysis using 293 human cells transfected with SpCas9 and exon 44, exon 45, exon 46, or exon 53 targeting sgRNAs. Sequences of the identified gRNA spacer sequences used to perform these experiments are listed in Table 2. For 4E43 and 4E45, the gRNA spacer sequences used were hDMD-E44g4, hDMD-E44g8, hDMD-E44g11, hDMD-E46g2, hDMD-E46g8, hDMD-E53g14, hDMD-E53g15, and hDMD-E53g23, and the gRNA scaffold sequence used in combination with each spacer sequence corresponds to SEQ ID NO: 147.

### DETAILED DESCRIPTION

DMD is a new mutation syndrome, and more than 4,000 independent causative mutations that have been identified in humans (world-wide web at dmd.nl). The majority of patient mutations carry deletions that cluster in a hotspot, and thus a therapeutic approach for skipping certain exon applies to large group of patients. The rationale of the exon skipping approach is based on the genetic difference between DMD and Becker muscular dystrophy (BMD) patients. In DMD patients, the reading frame of dystrophin mRNA is disrupted resulting in prematurely truncated, non-functional dystrophin proteins. BMD patients have mutations in the DMD gene that maintain the reading frame allowing the production of internally deleted, but partially functional dystrophins leading to much milder disease symptoms compared to DMD patients.

One the most common mutational hot spots in DMD is the genetic region between exons 44 and 51. Therapeutic approaches involving skipping or reframing of exon 44, exon 46, and exon 53 would treat approximately 18% of the DMD population. Here, the efficiency of CRISPR/Cas9 mediated correction of DMD mutations in patient-derived iPSCs is shown. To further assess the efficiency and optimize CRISPR/Cas9-mediated exon skipping in vivo, a mimic of the human “hot spot” region was generated in mouse models by deleting the exon 43, exon 45, or exon 52 using CRISPR/Cas9 system directed by two single guide RNAs (sgRNAs). The Δ43, Δ45, and Δ52 mouse models exhibit dystrophic myofibers, increased serum creatine kinase level, and reduced muscle function, thus providing a new set of representative models of DMD. These and other aspects of the disclosure are reproduced below.

Unless otherwise defined, all technical and scientific terms used herein have the same meaning as commonly understood by one of ordinary skill in the art to which this disclosure belongs. The terminology used in the detailed description herein is for the purpose of describing particular embodiments only and is not intended to be limiting.

All publications, patent applications, patents, GenBank or other accession numbers and other references mentioned herein are each incorporated by reference herein in their entirety.

The singular forms “a,” “an” and “the” are intended to include the plural forms as well, unless the context clearly indicates otherwise.

Furthermore, the terms “about” and “approximately” as used herein when referring to a measurable value such as an amount of the length of a polynucleotide or polypeptide sequence, dose, time, temperature, and the like, is meant to encompass variations of ±20%, ±10%, ±5%, ±1%, ±0.5%, or even ±0.1% of the specified amount.

Also as used herein, “and/or” refers to and encompasses any and all possible combinations of one or more of the associated listed items, as well as the lack of combinations when interpreted in the alternative (“or”).

Reference to a vector or other DNA sequences as “recombinant” merely acknowledges the operable linkage of DNA sequences which are not typically operably linked as isolated from or found in nature.

The terms “gRNA” and “sgRNA” are used interchangeably herein, and refer to a short synthetic RNA composed of a “spacer” (or “targeting”) sequence and a “scaffold” sequence. In some embodiments, the gRNA may further comprise a polyA tail.

In accordance with some embodiments of the disclosure, a “frameshift mutation” (or “frame-shift mutation” or “frameshift”) is caused by a deletion or insertion in a DNA sequence that shifts the reading frame of the DNA sequence.

In accordance with some embodiments of the disclosure, “exon skipping” (or “exon-skipping”) refers to a strategy which causes sections (e.g. mutated sections) of a gene to be “skipped” during RNA splicing, allowing the expression of a partially or fully functional protein.

Unless the context indicates otherwise, it is specifically intended that the various features described herein can be used in any combination.

### I. GENE EDITING SYSTEMS, E.G., CRISPR SYSTEMS

Provided herein are gene editing systems which produce an insertion, deletion, or replacement of DNA at a specific site in the genome of an organism or cell. In some embodiments, the genome editing systems introduce a loss of function mutation or a gain of function mutation. In some embodiments, the genome editing systems of the disclosure are capable of modulating splicing or causing a frameshift in a target DNA sequence. In some embodiments, the genome editing systems correct DNA mutations in vitro and/or in vivo.

The genome editing systems of the disclosure may comprise at least one nuclease (or catalytic domain thereof) and at least one gRNA, or nucleic acids encoding the at least one nuclease (or catalytic domain thereof) and the at least one gRNA. A sequence encoding the at least one nuclease and a sequence encoding the at least one gRNA may be delivered using the same vector (e.g., an AAV vector), or using different vectors (e.g., a first AAV vector for delivering the sequence encoding the nuclease, and a second AAV vector for delivering the sequence encoding the at least one gRNA).

In some embodiments, the nuclease is a Type II, Type V-A, Type V-B, Type V-C, Type V-U, Type VI-B nuclease. In some embodiments, the nuclease is a transcription activator-like effector nuclease (TALEN), a meganuclease, or a zinc-finger nuclease. In some embodiments, the nuclease is a Cas9, Cas12a (Cpf1), Cas12b, Cas12c, Tnp-B like, Cas13a (C2c2), Cas13b, or Cas14 nuclease. For example, in some embodiments, the nuclease is a Cas9 nuclease or a Cpf1 nuclease.

In some embodiments, the nuclease is a modified form or variant of a Cas9, Cas12a (Cpf1), Cas12b, Cas12c, Tnp-B like, Cas13a (C2c2), Cas13b, or Cas14 nuclease. In some embodiments, the nuclease is a modified form or variant of a TAL nuclease, a meganuclease, or a zinc-finger nuclease. A “modified” or “variant” nuclease is one that is, for example, truncated, fused to another protein (such as another nuclease), catalytically inactivated, etc. In some embodiments, the nuclease may have at least 80%, at least 85%, at least 90%, at least 95%, at least 98%, at least 99%, or 100% sequence identity to a naturally occurring Cas9, Cas12a (Cpf1), Cas12b, Cas12c, Tnp-B like, Cas13a (C2c2), Cas13b, Cas14 nuclease, or a TALEN, meganuclease, or zinc-finger nuclease.

In embodiments, the nuclease is a Cas9 nuclease derived from S. pyogenes (SpCas9). An exemplary SpCas9 sequence is provided in SEQ ID NO: 166. In some embodiments, the nuclease has at least 90%, at least 95%, at least 96%, at least 97%, at least 98%, or at least 99% sequence identity to SEQ ID NO: 166, shown below:

(SEQ ID NO: 166)
MDKKYSIGLDIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESILPKRNSDKLIARKKDWDPKKYGGFDSPTVAYSVLVVAKVE
KGKSKKLKSVKELLGITIMERSSFEKNPIDFLEAKGYKEVKKDLIIKLPK
YSLFELENGRKRMLASAGELQKGNELALPSKYVNFLYLASHYEKLKGSPE
DNEQKQLFVEQHKHYLDEIIEQISEFSKRVILADANLDKVLSAYNKHRDK
PIREQAENIIHLFTLTNLGAPAAFKYFDTTIDRKRYTSTKEVLDATLIHQ
SITGLYETRIDLSQLGGD

In embodiments, the nuclease is a Cas9 derived from S. aureus (SaCas9). An exemplary SaCas9 sequence is provided in SEQ ID NO: 167. In some embodiments, the nuclease has at least 90%, at least 95%, at least 96%, at least 97%, at least 98%, or at least 99% sequence identity to SEQ ID NO: 167, shown below:

(SEQ ID NO: 167)
MKRNYILGLDIGITSVGYGIIDYETRDVIDAGVRLFKEANVENNEGRRSK
RGARRLKRRRRHRIQRVKKLLFDYNLLTDHSELSGINPYEARVKGLSQKL
SEEEFSAALLHLAKRRGVHNVNEVEEDTGNELSTKEQISRNSKALEEKYV
AELQLERLKKDGEVRGSINRFKTSDYVKEAKQLLKVQKAYHQLDQSFIDT
YIDLLETRRTYYEGPGEGSPFGWKDIKEWYEMLMGHCTYFPEELRSVKYA
YNADLYNALNDLNNLVITRDENEKLEYYEKFQIIENVFKQKKKPTLKQIA
KEILVNEEDIKGYRVTSTGKPEFTNLKVYHDIKDITARKEIIENAELLDQ
IAKILTIYQSSEDIQEELTNLNSELTQEEIEQISNLKGYTGTHNLSLKAI
NLILDELWHTNDNQIAIFNRLKLVPKKVDLSQQKEIPTTLVDDFILSPVV
KRSFIQSIKVINAIIKKYGLPNDIIIELAREKNSKDAQKMINEMQKRNRQ
TNERIEEIIRTTGKENAKYLIEKIKLHDMQEGKCLYSLEAIPLEDLLNNP
FNYEVDHIIPRSVSFDNSFNNKVLVKQEENSKKGNRTPFQYLSSSDSKIS
YETFKKHILNLAKGKGRISKTKKEYLLEERDINRFSVQKDFINRNLVDTR
YATRGLMNLLRSYFRVNNLDVKVKSINGGFTSFLRRKWKFKKERNKGYKH
HAEDALIIANADFIFKEWKKLDKAKKVMENQMFEEKQAESMPEIETEQEY
KEIFITPHQIKHIKDFKDYKYSHRVDKKPNRELINDTLYSTRKDDKGNTL
IVNNLNGLYDKDNDKLKKLINKSPEKLLMYHHDPQTYQKLKLIMEQYGDE
KNPLYKYYEETGNYLTKYSKKDNGPVIKKIKYYGNKLNAHLDITDDYPNS
RNKVVKLSLKPYRFDVYLDNGVYKFVTVKNLDVIKKENYYEVNSKCYEEA
KKLKKISNQAEFIASFYNNDLIKINGELYRVIGVNNDLLNRIEVNMIDIT
YREYLENMNDKRPPRIIKTIASKTQSIKKYSTDILGNLYEVKSKKHPQII

In embodiments, the nuclease is a Cpf1 enzyme from Acidaminococcus (species BV3L6, UniProt Accession No. U2UMQ6). For example, the Cpf1 enzyme may have the sequence set forth below (SEQ ID NO: 168), or a sequence with at least 90%, at least 95%, at least 96%, at least 97%, at least 98%, or at least 99% sequence identity thereto:

(SEQ ID NO: 168)
MTQFEGFTNLYQVSKTLRFELIPQGKTLKHIQEQGFIEEDKARNDHYKEL
KPIIDRIYKTYADQCLQLVQLDWENLSAAIDSYRKEKTEETRNALIEEQA
TYRNAIHDYFIGRTDNLTDAINKRHAEIYKGLFKAELFNGKVLKQLGTVT
TTEHENALLRSFDKFTTYFSGFYENRKNVFSAEDISTAIPHRIVQDNFPK
FKENCHIFTRLITAVPSLREHFENVKKAIGIFVSTSIEEVFSFPFYNQLL
TQTQIDLYNQLLGGISREAGTEKIKGLNEVLNLAIQKNDETAHIIASLPH
RFIPLFKQILSDRNTLSFILEEFKSDEEVIQSFCKYKTLLRNENVLETAE
ALFNELNSIDLTHIFISHKKLETISSALCDHWDTLRNALYERRISELTGK
ITKSAKEKVQRSLKHEDINLQEIISAAGKELSEAFKQKTSEILSHAHAAL
DQPLPTTLKKQEEKEILKSQLDSLLGLYHLLDWFAVDESNEVDPEFSARL
TGIKLEMEPSLSFYNKARNYATKKPYSVEKFKLNFQMPTLASGWDVNKEK
NNGAILFVKNGLYYLGIMPKQKGRYKALSFEPTEKTSEGFDKMYYDYFPD
AAKMIPKCSTQLKAVTAHFQTHTTPILLSNNFIEPLEITKEIYDLNNPEK
EPKKFQTAYAKKTGDQKGYREALCKWIDFTRDFLSKYTKTTSIDLSSLRP
SSQYKDLGEYYAELNPLLYHISFQRIAEKEIMDAVETGKLYLFQIYNKDF
AKGHHGKPNLHTLYWTGLFSPENLAKTSIKLNGQAELFYRPKSRMKRMAH
RLGEKMLNKKLKDQKTPIPDTLYQELYDYVNHRLSHDLSDEARALLPNVI
TKEVSHEIIKDRRFTSDKFFFHVPITLNYQAANSPSKFNQRVNAYLKEHP
ETPIIGIDRGERNLIYITVIDSTGKILEQRSLNTIQQFDYQKKLDNREKE
RVAARQAWSVVGTIKDLKQGYLSQVIHEIVDLMIHYQAVVVLENLNFGFK
SKRTGIAEKAVYQQFEKMLIDKLNCLVLKDYPAEKVGGVLNPYQLTDQFT
SFAKMGTQSGFLFYVPAPYTSKIDPLTGFVDPFVWKTIKNHESRKHFLEG
FDFLHYDVKTGDFILHFKMNRNLSFQRGLPGFMPAWDIVFEKNETQFDAK
GTPFIAGKRIVPVIENHRFTGRYRDLYPANELIALLEEKGIVFRDGSNIL
PKLLENDDSHAIDTMVALIRSVLQMRNSNAATGEDYINSPVRDLNGVCFD
SRFQNPEWPMDADANGAYHIALKGQLLLNHLKESKDLKLQNGISNQDWLA
YIQELRN

In some embodiments, the nuclease is a Cpf1 enzyme from Lachnospiraceae (species ND2006, UniProt Accession No. A0A182DWE3). An exemplary Lachnospiraceae Cpf1 sequence is provided in SEQ ID NO: 169. In some embodiments, the nuclease has at least 90%, at least 95%, at least 96%, at least 97%, at least 98%, or at least 99% sequence identity to SEQ ID NO: 169, which is provided below:

(SEQ ID NO: 169)
AASKLEKFTNCYSISKTLRFKAIPVGKTQENIDNKRLLVEDEKRAEDYKG
VKKLLDRYYLSFINDVLHSIKLKNLNNYISLFRKKTRTEKENKELENLEI
NLRKEIAKAFKGAAGYKSLFKKDIIETILPEAADDKDEIALVNSFNGFTT
AFTGFFDNRENMFSEEAKSTSIAFRCINENLTRYISNMDIFEKVDAIFDK
HEVQEIKEKILNSDYDVEDFFEGEFFNFVLTQEGIDVYNAIIGGFVTESG
EKIKGLNEYINLYNAKTKQALPKFKPLYKQVLSDRESLSFYGEGYTSDEE
VLEVFRNTLNKNSEIFSSIKKLEKLFKNFDEYSSAGIFVKNGPAISTISK
DIFGEWNLIRDKWNAEYDDIHLKKKAVVTEKYEDDRRKSFKKIGSFSLEQ
LQEYADADLSVVEKLKEIIIQKVDEIYKVYGSSEKLFDADFVLEKSLKKN
DAVVAIMKDLLDSVKSFENYIKAFFGEGKETNRDESFYGDEVLAYDILLK
VDHIYDAIRNYVTQKPYSKDKFKLYFQNPQFMGGWDKDKETDYRATILRY
GSKYYLAIMDKKYAKCLQKIDKDDVNGNYEKINYKLLPGPNKMLPKVFFS
KKWMAYYNPSEDIQKIYKNGTFKKGDMFNLNDCHKLIDFFKDSISRYPKW
SNAYDFNFSETEKYKDIAGFYREVEEQGYKVSFESASKKEVDKLVEEGKL
YMFQIYNKDFSDKSHGTPNLHTMYFKLLFDENNHGQIRLSGGAELFMRRA
SLKKEELVVHPANSPIANKNPDNPKKTTTLSYDVYKDKRFSEDQYELHIP
IAINKCPKNIFKINTEVRVLLKHDDNPYVIGIDRGERNLLYIVVVDGKGN
IVEQYSLNEIINNFNGIRIKTDYHSLLDKKEKERFEARQNWTSIENIKEL
KAGYISQVVHKICELVEKYDAVIALEDLNSGFKNSRVKVEKQVYQKFEKM
LIDKLNYMVDKKSNPCATGGALKGYQITNKFESFKSMSTQNGFIFYIPAW
LTSKIDPSTGFVNLLKTKYTSIADSKKFISSFDRIMYVPEEDLFEFALDY
KNFSRTDADYIKKWKLYSYGNRIRIFAAAKKNNVFAWEEVCLTSAYKELF
NKYGINYQQGDIRALLCEQSDKAFYSSFMALMSLMLQMRNSITGRTDVDF
LISPVKNSDGIFYDSRNYEAQENAILPKNADANGAYNIARKVLWAIGQFK
KAEDEKLDKVKIAISNKEWLEYAQTSVK

In some embodiments, a sequence encoding the nuclease is codon optimized for expression in mammalian cells. In some embodiments, the sequence encoding the nuclease is codon optimized for expression in human cells or mouse cells.

In some embodiments, the disclosure provides a nucleic acid comprising a sequence encoding a single guide RNA (sgRNA) comprising a spacer sequence and a scaffold sequence.

A. Spacer

A spacer sequence is a short nucleic acid sequence used to target a nuclease (e.g., a Cas9 nuclease) to a specific nucleotide region of interest (e.g., a genomic DNA sequence to be cleaved).

In some embodiments, the spacer may be about 17-24 base pairs in length, such as about 20 base pairs in length. In some embodiments, the spacer may be about 15, about 16, about 17, about 18, about 19, about 20, about 21, about 22, about 23, about 24, about 25, about 26, about 27, about 28, about 29, or about 30 base pairs in length. In some embodiments, the spacer may be at least 15, at least 16, at least 17, at least 18, at least 19, at least 20, at least 21, at least 22, at least 23, at least 24, at least 25, at least 26, at least 27, at least 28, at least 29, or at least 30 base pairs in length. In some embodiments, the spacer may be 15, 16, 17, 18, 19, 20, 21, 22, 23, 24, 25, 26, 27, 28, 29, or 30 base pairs in length. In some embodiments, the spacer sequence has between about 40% to about 80% GC content.

In some embodiments, the spacer targets a site that immediately precedes a 5′ protospacer adjacent motif (PAM). The PAM sequence may be selected based on the desired nuclease. For example, the PAM sequence may be any one of the PAM sequences shown in Table 1 below, wherein N refers to any nucleic acid, R refers to A or G, Y refers to C or T, W refers to A or T, and V refers to A or C or G.

TABLE 1
Exemplary Nucleases and PAM sequences

| PAM | | | |
|---|---|---|---|
| sequence (5′ | SEQ | | |
| to 3′) | ID NO: | Nuclease | Isolated from |
| NGG | — | SpCas9 | Streptococcus pyogenes |
| NGRRT or | 128, 129 | SaCas9 | Staphylococcus aureus |
| NGRRN | | | |
| NNNNGATT | 130 | NmeCas9 | Neisseria meningitidis |
| NNNNRYAC | 131 | CjCas9 | Campylobacter jejuni |
| NNAGAAW | 132 | StCas9 | Streptococcus thermophilus |
| TTTV | 133 | LbCpf1 | Lachnospiraceae bacterium |
| TTTV | 134 | AsCpf1 | Acidaminococcus sp. |

In some embodiments, a spacer may target a sequence of a mammalian gene, such as a human gene. In some embodiments, the spacer may target a mutant gene. In some embodiments, the spacer may target a coding sequence. In some embodiments, the spacer targets the dystrophin (DMD) gene. An exemplary wild-type dystrophin sequence includes the human DNA sequence (see GenBank Accession NO. NC_000023.11), located on the human X chromosome, which codes for the protein dystrophin (GenBank Accession No. AAA53189), the sequence of which is reproduced below:

| (SEQ ID NO: 330) | |
|---|---|
| 1 | MLWWEEVEDC YEREDVQKKT FTKWVNAQFS KFGKQHIENL FSDLQDGRRL LDLLEGLTGQ |
| 61 | KLPKEKGSTR VHALNNVNKA LRVLQNNNVD LVNIGSTDIV DGNHKLTLGL IWNIILHWQV |
| 121 | KNVMKNIMAG LQQTNSEKIL LSWVRQSTRN YPQVNVINFT TSWSDGLALN ALIHSHRPDL |
| 181 | FDWNSVVCQQ SATQRLEHAF NIARYQLGIE KLLDPEDVDT TYPDKKSILM YITSLFQVLP |
| 241 | QQVSIEAIQE VEMLPRPPKV TKEEHFQLHH QMHYSQQITV SLAQGYERTS SPKPRFKSYA |
| 301 | YTQAAYVTTS DPTRSPFPSQ HLEAPEDKSF GSSLMESEVN LDRYQTALEE VLSWLLSAED |
| 361 | TLQAQGEISN DVEVVKDQFH THEGYMMDLT AHQGRVGNIL QLGSKLIGTG KLSEDEETEV |
| 421 | QEQMNLLNSR WECLRVASME KQSNLHRVLM DLQNQKLKEL NDWLTKTEER TRKMEEEPLG |
| 481 | PDLEDLKRQV QQHKVLQEDL EQEQVRVNSL THMVVVVDES SGDHATAALE EQLKVLGDRW |
| 541 | ANICRWTEDR WVLLQDILLK WQRLTEEQCL FSAWLSEKED AVNKIHTTGF KDQNEMLSSL |
| 601 | QKLAVLKADL EKKKQSMGKL YSLKQDLLST LKNKSVTQKT EAWLDNFARC WDNLVQKLEK |
| 661 | STAQISQAVT TTQPSLTQTT VMETVTTVTT REQILVKHAQ EELPPPPPQK KRQITVDSEI |
| 721 | RKRLDVDITE LHSWITRSEA VLQSPEFAIF RKEGNFSDLK EKVNAIEREK AEKFRKLQDA |
| 781 | SRSAQALVEQ MVNEGVNADS IKQASEQLNS RWIEFCQLLS ERLNWLEYQN NIIAFYNQLQ |
| 841 | QLEQMTTTAE NWLKIQPTTP SEPTAIKSQL KICKDEVNRL SGLQPQIERL KIQSIALKEK |
| 901 | GQGPMFLDAD EVAFTNHFKQ VFSDVQAREK ELQTIFDTLP PMRYQETMSA IRTWVQQSET |
| 961 | KLSIPQLSVT DYEIMEQRLG ELQALQSSLQ EQQSGLYYLS TTVKEMSKKA PSEISRKYQS |
| 1021 | EFEEIEGRWK KLSSQLVEHC QKLEEQMNKL RKIQNHIQTL KKWMAEVDVF LKEEWPALGD |
| 1081 | SEILKKQLKQ CRLLVSDIQT IQPSLNSVNE GGQKIKNEAE PEFASRLETE LKELNTQWDH |
| 1141 | MCQQVYARKE ALKGGLEKTV SLQKDLSEMH EWMTQAEEEY LERDFEYKTP DELQKAVEEM |
| 1201 | KRAKEEAQQK EAKVKLLTES VNSVIAQAPP VAQEALKKEL ETLTTNYQWL CTRLNGKCKT |
| 1261 | LEEVWACWHE LLSYLEKANK WLNEVEFKLK TTENIPGGAE EISEVLDSLE NLMRHSEDNP |
| 1321 | NQIRILAQTL TDGGVMDELI NEELETFNSR WRELHEEAVR RQKLLEQSIQ SAQETEKSLH |
| 1381 | LIQESLTFID KQLAAYIADK VDAAQMPQEA QKIQSDLTSH EISLEEMKKH NQGKEAAQRV |
| 1441 | LSQIDVAQKK LQDVSMKFRL FQKPANFELR LQESKMILDE VKMHLPALET KSVEQEVVQS |
| 1501 | QLNHCVNLYK SLSEVKSEVE MVIKTGRQIV QKKQTENPKE LDERVTALKL HYNELGAKVT |
| 1561 | ERKQQLEKCL KLSRKMRKEM NVLTEWLAAT DMELTKRSAV EGMPSNLDSE VAWGKATQKE |
| 1621 | IEKQKVHLKS ITEVGEALKT VLGKKETLVE DKLSLLNSNW IAVTSRAEEW LNLLLEYQKH |
| 1681 | METFDQNVDH ITKWIIQADT LLDESEKKKP QQKEDVLKRL KAELNDIRPK VDSTRDQAAN |
| 1741 | LMANRGDHCR KLVEPQISEL NHRFAAISHR IKTGKASIPL KELEQFNSDI QKLLEPLEAE |
| 1801 | IQQGVNLKEE DFNKDMNEDN EGTVKELLQR GDNLQQRITD ERKREEIKIK QQLLQTKHNA |
| 1861 | LKDLRSQRRK KALEISHQWY QYKRQADDLL KCLDDIEKKL ASLPEPRDER KIKEIDRELQ |
| 1921 | KKKEELNAVR RQAEGLSEDG AAMAVEPTQI QLSKRWREIE SKFAQFRRLN FAQIHTVREE |
| 1981 | TMMVMTEDMP LEISYVPSTY LTEITHVSQA LLEVEQLLNA PDLCAKDFED LFKQEESLKN |
| 2041 | IKDSLQQSSG RIDIIHSKKT AALQSATPVE RVKLQEALSQ LDFQWEKVNK MYKDRQGRFD |
| 2101 | RSVEKWRRFH YDIKIFNQWL TEAEQFLRKT QIPENWEHAK YKWYLKELQD GIGQRQTVVR |
| 2161 | TLNATGEEII QQSSKTDASI LQEKLGSLNL RWQEVCKQLS DRKKRLEEQK NILSEFQRDL |
| 2221 | NEFVLWLEEA DNIASIPLEP GKEQQLKEKL EQVKLLVEEL PLRQGILKQL NETGGPVLVS |
| 2281 | APISPEEQDK LENKLKQTNL QWIKVSRALP EKQGEIEAQI KDLGQLEKKL EDLEEQLNHL |
| 2341 | LLWLSPIRNQ LEIYNQPNQE GPFDVQETEI AVQAKQPDVE EILSKGQHLY KEKPATQPVK |
| 2401 | RKLEDLSSEW KAVNRLLQEL RAKQPDLAPG LTTIGASPTQ TVTLVTQPVV TKETAISKLE |
| 2461 | MPSSLMLEVP ALADFNRAWT ELTDWLSLLD QVIKSQRVMV GDLEDINEMI IKQKATMQDL |
| 2521 | EQRRPQLEEL ITAAQNLKNK TSNQEARTII TDRIERIQNQ WDEVQEHLQN RRQQLNEMLK |
| 2581 | DSTQWLEAKE EAEQVLGQAR AKLESWKEGP YTVDAIQKKI TETKQLAKDL RQWQTNVDVA |
| 2641 | NDLALKLLRD YSADDTRKVH MITENINASW RSIHKRVSER EAALEETHRL LQQFPLDLEK |
| 2701 | FLAWLTEAET TANVLQDATR KERLLEDSKG VKELMKQWQD LQGEIEAHTD VYHNLDENSQ |
| 2761 | KILRSLEGSD DAVLLQRRLD NMNFKWSELR KKSLNIRSHL EASSDQWKRL HLSLQELLVW |
| 2821 | LQLKDDELSR QAPIGGDFPA VQKQNDVHRA FKRELKTKEP VIMSTLETVR IFLTEQPLEG |
| 2881 | LEKLYQEPRE LPPEERAQNV TRLLRKQAEE VNTEWEKLNL HSADWQRKID ETLERLQELQ |
| 2941 | EATDELDLKL RQAEVIKGSW QPVGDLLIDS LQDHLEKVKA LRGEIAPLKE NVSHVNDLAR |
| 3001 | QLTTLGIQLS PYNLSTLEDL NTRWKLLQVA VEDRVRQLHE AHRDFGPASQ HFLSTSVQGP |
| 3061 | WERAISPNKV PYYINHETQT TCWDHPKMTE LYQSLADLNN VRFSAYRTAM KLRRLQKALC |
| 3121 | LDLLSLSAAC DALDQHNLKQ NDQPMDILQI INCLTTIYDR LEQEHNNLVN VPLCVDMCLN |
| 3181 | WLLNVYDTGR TGRIRVLSFK TGIISLCKAH LEDKYRYLFK QVASSTGFCD QRRLGLLLHD |
| 3241 | SIQIPRQLGE VASFGGSNIE PSVRSCFQFA NNKPEIEAAL FLDWMRLEPQ SMVWLPVLHR |
| 3301 | VAAAETAKHQ AKCNICKECP IIGFRYRSLK HFNYDICQSC FFSGRVAKGH KMHYPMVEYC |
| 3361 | TPTTSGEDVR DFAKVLKNKF RTKRYFAKHP RMGYLPVQTV LEGDNMETPV TLINFWPVDS |
| 3421 | APASSPQLSH DDTHSRIEHY ASRLAEMENS NGSYLNDSIS PNESIDDEHL LIQHYCQSLN |
| 3481 | QDSPLSQPRS PAQILISLES EERGELERIL ADLEEENRNL QAEYDRLKQQ HEHKGLSPLP |
| 3541 | SPPEMMPTSP QSPRDAELIA EAKLLRQHKG RLEARMQILE DHNKQLESQL HRLRQLLEQP |
| 3601 | QAEAKVNGTT VSSPSTSLQR SDSSQPMLLR VVGSQTSDSM GEEDLLSPPQ DTSTGLEEVM |
| 3661 | EQLNNSFPSS RGRNTPGKPM REDTM.. |

In some embodiments, the spacer sequence targets a sequence of the DMD gene. In some embodiments, the spacer targets an exon of the DMD gene. In some embodiments, the spacer targets exon 43, exon 44, exon 46, exon 50 or exon 53 of the DMD gene.

In some embodiments, the spacer may have a sequence of any one of SEQ ID NOs: 135-146, 170-260, 337-339, or 617 (shown in Table 2 below). In some embodiments, a spacer may have a sequence at least 90%, at least 95%, at least 96%, at least 97%, at least 98%, or at least 99% identical to the sequence of any one of SEQ ID NOs: 135-146, 170-260, 337-339, or 617. In some embodiments, a spacer may have a sequence of any one of the spacers shown in Table 2, or a sequence that is at least 90%, at least 95%, at least 96%, at least 97%, at least 98%, or at least 99% identical thereto.

TABLE 2
Exemplary spacer sequences for targeting an exon
of the human DMD gene

| sgRNA spacer | Exon | | | SEQ ID |
|---|---|---|---|---|
| name | targeted | Species | sgRNA spacer sequence | NO |
| hDMD-E43g1 | 43 | Human | GTTTTAAAATTTTTATATTA | 189 |
| hDMD-E43g2 | 43 | Human | TTTTTATATTACAGAATATAA | 190 |
| hDMD-E43g3 | 43 | Human | ATATTACAGAATATAAAAGA | 191 |
| hDMD-E43g5 | 43 | Human | AAATGTACAAGGACCGACAA | 188 |
| hDMD-E43g4 | 43 | Human | TATGTGTTACCTACCCTTGT | 135 |
| hDMD-E43g6 | 43 | Human | GTACAAGGACCGACAAGGGT | 136 |
| hDMD-E44g1 | 44 | Human | ATCCATATGCTTTTACCTGC | 137 |
| hDMD-E44g2 | 44 | Human | gatccatatgcttttACCTG | 192 |
| hDMD-E44g3 | 44 | Human | CAGATCTGTCAAATCGCCTG | 193 |
| hDMD-E44g4 | 44 | Human | TAAATACAAATGGTATCTTA | 138 |
| hDMD-E44g5 | 44 | Human | AGATCTGTCAAATCGCCTGC | 139 |
| hDMD-E44g6 | 44 | Human | ACAGATCTGTTGAGAAATGG | 140 |
| hDMD-E44g7 | 44 | Human | TTAGCATGTTCCCAATTCTC | 141 |
| hDMD-E44g8 | 44 | Human | GGGAACATGCTAAATACAAA | 142 |
| hDMD-E44g9 | 44 | Human | TTGACAGATCTGTTGAGAAA | 143 |
| hDMD- | 44 | Human | AGACACAAATTCCTGAGAAT | 144 |
| E44g10 | | | | |
| hDMD- | 44 | Human | GACACAAATTCCTGAGAATT | 145 |
| E44g11 | | | | |
| hDMD- | 44 | Human | CGCctgcaggtaaaagcata | 194 |
| E44g12 | | | | |
| hDMD- | 44 | Human | tttacctgcagGCGATTTGA | 195 |
| E44g13 | | | | |
| hDMD- | 44 | Human | gGCGATTTGACAGATCTGTT | 196 |
| E44g14 | | | | |
| hDMD- | 44 | Human | ATTTAATCAGTGGCTAACAG | 197 |
| E44g15 | | | | |
| hDMD- | 44 | Human | AGAAACTGTTCAGCTTCTGT | 198 |
| E44g16 | | | | |
| hDMD- | 44 | Human | AGTGGCTAACAGAAGCTGAA | 199 |
| E44g17 | | | | |
| hDMD- | 44 | Human | AAGCTGAACAGTTTCTCAGA | 200 |
| E44g18 | | | | |
| hDMD- | 44 | Human | TTTAGCATGTTCCCAATTCT | 201 |
| E44g19 | | | | |
| hDMD- | 44 | Human | CTTAAGATACCATTTGTATT | 202 |
| E44g20 | | | | |
| hDMD- | 44 | Human | CTAAATACAAATGGTATCTT | 203 |
| E44g21 | | | | |
| hDMD- | 44 | Human | TACAAATGGTATCTTAAGgt | 204 |
| E44g22 | | | | |
| hDMD- | 44 | Human | ACAAATCAAAGACTTACCTT | 146 |
| E44g23 | | | | |
| hDMD-E45g4 | 45 | Human | atcttacagGAACTCCAGGA | 617 |
| hDMD-E46g1 | 46 | Human | ttattcttctttctccagGC | 205 |
| hDMD-E46g2 | 46 | Human | AATTTTATTCTTCTTTCTCC | 170 |
| hDMD-E46g5 | 46 | Human | ATTCTTTTGTTCTTCTAGCC | 171 |
| hDMD-E46g6 | 46 | Human | AAATGAATTTGTTTTATGGT | 172 |
| hDMD-E46g7 | 46 | Human | TGAATTTGTTTTATGGTTGG | 173 |
| hDMD-E46g8 | 46 | Human | AGAAAAGCTTGAGCAAGTCA | 174 |
| hDMD-E46g9 | 46 | Human | TTCTTCTAGCCTGGAGAAAG | 206 |
| hDMD- | 46 | Human | CAATTTTATTCTTCTTTCTC | 207 |
| E46g10 | | | | |
| hDMD- | 46 | Human | TTGTTCTTCTAGCCTGGAGA | 208 |
| E46g11 | | | | |
| hDMD- | 46 | Human | TCTTTTGTTCTTCTAGCCTG | 209 |
| E46g12 | | | | |
| hDMD- | 46 | Human | TTCTTCTTTCTCCAGGCTAG | 210 |
| E46g13 | | | | |
| hDMD- | 46 | Human | ACTTGCTCAAGCTTTTCTTT | 211 |
| E46g14 | | | | |
| hDMD- | 46 | Human | ACTAAAAGAAAAGCTTGAGC | 212 |
| E46g15 | | | | |
| hDMD- | 46 | Human | AAAATTACCTTGACTTGCTC | 213 |
| E46g16 | | | | |
| hDMD- | 46 | Human | AAGAAAAGCTTGAGCAAGTC | 214 |
| E46g17 | | | | |
| hDMD- | 46 | Human | TCTCCAGGCTAGAAGAACAA | 337 |
| E46g18 | | | | |
| hDMD- | 46 | Human | AGAACAAAAGAATATCTTGT | 338 |
| E46g19 | | | | |
| hDMD- | 46 | Human | TATCTTGTCAGAATTTCAAA | 339 |
| E46g20 | | | | |
| hDMD-E50g1 | 50 | Human | TGTATGCTTTTCTGTTAAAG | 175 |
| hDMD-E50g2 | 50 | Human | ATGTGTATGCTTTTCTGTTA | 215 |
| hDMD-E50g3 | 50 | Human | GTGTATGCTTTTCTGTTAAA | 216 |
| hDMD-E50g4 | 50 | Human | ATGCTTTTCTGTTAAAGAGG | 217 |
| hDMD-E50g5 | 50 | Human | TCTTCTAACTTCCTCTTTAA | 218 |
| hDMD-E50g6 | 50 | Human | TAACTTCCTCTTTAACAGAA | 219 |
| hDMD-E50g7 | 50 | Human | TTTTCTGTTAAAGAGGAAGT | 220 |
| hDMD-E50g8 | 50 | Human | TCTGTTAAAGAGGAAGTTAG | 221 |
| hDMD-E50g9 | 50 | Human | AAGAGGAAGTTAGAAGATCT | 222 |
| hDMD- | 50 | Human | AGTTAGAAGATCTGAGCTCT | 223 |
| E50g10 | | | | |
| hDMD- | 50 | Human | TAGAAGATCTGAGCTCTGAG | 224 |
| E50g11 | | | | |
| hDMD- | 50 | Human | AGATCTGAGCTCTGAGTGGA | 225 |
| E50g12 | | | | |
| hDMD- | 50 | Human | ACCGCCTTCCACTCAGAGCT | 226 |
| E50g13 | | | | |
| hDMD- | 50 | Human | GGTTTACCGCCTTCCACTCA | 227 |
| E50g14 | | | | |
| hDMD- | 50 | Human | AAGCAGCCTGACCTAGCTCC | 228 |
| E50g15 | | | | |
| hDMD- | 50 | Human | GTCAGTCCAGGAGCTAGGTC | 229 |
| E50g16 | | | | |
| hDMD- | 50 | Human | GGTCAGTCCAGGAGCTAGGT | 230 |
| E50g17 | | | | |
| hDMD- | 50 | Human | TAGTGGTCAGTCCAGGAGCT | 231 |
| E50g18 | | | | |
| hDMD- | 50 | Human | ATAGTGGTCAGTCCAGGAGC | 232 |
| E50g19 | | | | |
| hDMD- | 50 | Human | TCCAATAGTGGTCAGTCCAG | 233 |
| E50g20 | | | | |
| hDMD- | 50 | Human | GCTCCAATAGTGGTCAGTCC | 234 |
| E50g21 | | | | |
| hDMD- | 50 | Human | TTACAGGCTCCAATAGTGGT | 235 |
| E50g22 | | | | |
| hDMD- | 50 | Human | ATACTTACAGGCTCCAATAG | 236 |
| E50g23 | | | | |
| hDMD- | 50 | Human | AGTATACTTACAGGCTCCAA | 237 |
| E50g24 | | | | |
| hDMD- | 50 | Human | GCTCCTGGACTGACCACTAT | 238 |
| E50g25 | | | | |
| hDMD- | 50 | Human | TCCTGGACTGACCACTATTG | 239 |
| E50g26 | | | | |
| hDMD- | 50 | Human | TGACCACTATTGGAGCCTGT | 240 |
| E50g27 | | | | |
| hDMD- | 50 | Human | ATGGGATCCAGTATACTTAC | 241 |
| E50g28 | | | | |
| hDMD- | 50 | Human | AATGGGATCCAGTATACTTA | 242 |
| E50g29 | | | | |
| hDMD- | 50 | Human | ATTGGAGCCTGTAAGTATAC | 243 |
| E50g30 | | | | |
| hDMD-E51g4 | 51 | Human | TCATCTCGTTGATATCCTCA | 244 |
| hDMD-E51g5 | 51 | Human | CGAGATGATCATCAAGCAGA | 245 |
| hDMD-E51g6 | 51 | Human | GTGACCTTGAGGATATCAAC | 246 |
| hDMD-E51g7 | 51 | Human | TCAACGAGATGATCATCAAG | 247 |
| hDMD-E51g8 | 51 | Human | ACGAGATGATCATCAAGCAG | 248 |
| hDMD-E53g1 | 53 | Human | ATTTATTTTTCCTTTTATTC | 249 |
| hDMD-E53g2 | 53 | Human | TTTCCTTTTATTCTAGTTGA | 250 |
| hDMD-E53g3 | 53 | Human | TGATTCTGAATTCTTTCAAC | 251 |
| hDMD-E53g4 | 53 | Human | AAAGAAAATCACAGAAACCA | 176 |
| hDMD-E53g5 | 53 | Human | AAAATCACAGAAACCAAGGT | 252 |
| hDMD-E53g6 | 53 | Human | GGTATCTTTGATACTAACCT | 177 |
| hDMD-E53g7 | 53 | Human | TGAAAGAATTCAGAATCAGT | 178 |
| hDMD-E53g8 | 53 | Human | ACTGTTGCCTCCGGTTCTGA | 179 |
| hDMD-E53g9 | 53 | Human | TACAAGAACACCTTCAGAAC | 180 |
| hDMD- | 53 | Human | AAGAACACCTTCAGAACCGG | 181 |
| E53g10 | | | | |
| hDMD- | 53 | Human | TTTCATTCAACTGTTGCCTC | 182 |
| E53g11 | | | | |
| hDMD- | 53 | Human | TGTTAAAGGATTCAACACAA | 183 |
| E53g12 | | | | |
| hDMD- | 53 | Human | AAAGGATTCAACACAATGGC | 184 |
| E53g13 | | | | |
| hDMD- | 53 | Human | AATTCAGAATCAGTGGGATG | 185 |
| E53g14 | | | | |
| hDMD- | 53 | Human | TTGAAAGAATTCAGAATCAG | 186 |
| E53g15 | | | | |
| hDMD- | 53 | Human | ACAGTTGAATGAAATGTTAA | 253 |
| E53g16 | | | | |
| hDMD- | 53 | Human | ACCTTCAGAACCGGAGGCAA | 254 |
| E53g17 | | | | |
| hDMD- | 53 | Human | AATTCTTTCAActagaataa | 255 |
| E53g18 | | | | |
| hDMD- | 53 | Human | ttattctagTTGAAAGAATT | 256 |
| E53g19 | | | | |
| hDMD- | 53 | Human | tagTTGAAAGAATTCAGAAT | 257 |
| E53g20 | | | | |
| hDMD- | 53 | Human | ATGAAGTACAAGAACACCTT | 258 |
| E53g21 | | | | |
| hDMD- | 53 | Human | AACTGTTGCCTCCGGTTCTG | 259 |
| E53g22 | | | | |
| hDMD- | 53 | Human | CAAGAACACCTTCAGAACCG | 187 |
| E53g23 | | | | |
| hDMD- | 53 | Human | AACAGTTGAATGAAATGTTA | 260 |
| E53g24 | | | | |

In some embodiments, the spacer may have a sequence of any one of SEQ ID NOs: 261-329 (shown in Table 3 below). In some embodiments, a spacer may have a sequence at least 90%, at least 95%, at least 96%, at least 97%, at least 98%, or at least 99% identical to the sequence of any one of SEQ ID NOs: 261-329. In some embodiments, a spacer may have a sequence of any one of the spacers shown in Table 3, or a sequence that is at least 90%, at least 95%, at least 96%, at least 97%, at least 98%, or at least 99% identical thereto.

TABLE 3
Exemplary spacer sequences for targeting an exon of
the mouse DMD gene

| sgRNA spacer | Exon | | | SEQ ID |
|---|---|---|---|---|
| name | targeted | Species | sgRNA spacer sequence | NO |
| mDMD-E43g1 | 43 | Mouse | ATTTGCAACAAATCTCAGGT | 261 |
| mDMD-E43g2 | 43 | Mouse | AGAATGTACAAGGAACGACA | 262 |
| mDMD-E43g3 | 43 | Mouse | GAATGTACAAGGAACGACAA | 263 |
| mDMD-E43g4 | 43 | Mouse | GTACAAGGAACGACAAGGGT | 264 |
| mDMD-E44g1 | 44 | Mouse | catccatgttttcaaattat | 265 |
| mDMD-E44g2 | 44 | Mouse | TCGACAGATCAGTTGAAAAA | 266 |
| mDMD-E44g3 | 44 | Mouse | AGACACAAAATCCTGAAAAC | 267 |
| mDMD-E44g4 | 44 | Mouse | TTAGCATGTTCCCAGTTTTC | 268 |
| mDMD-E44g5 | 44 | Mouse | GACACAAAATCCTGAAAACT | 269 |
| mDMD-E44g6 | 44 | Mouse | GGGAACATGCTAAATACAAA | 270 |
| mDMD-E44g7 | 44 | Mouse | TAAATACAAATGGTATCTTA | 271 |
| mDMD-E44g8 | 44 | Mouse | tcatccatgttttcaaatta | 272 |
| mDMD-E44g9 | 44 | Mouse | tcaaattatagGCGATTCGA | 273 |
| mDMD- | 44 | Mouse | AAAAACTGTTCAACTTCATT | 274 |
| E44g10 | | | | |
| mDMD- | 44 | Mouse | AATGGCTGAATGAAGTTGAA | 275 |
| E44g11 | | | | |
| mDMD- | 44 | Mouse | AAGTTGAACAGTTTTTCAAA | 276 |
| E44g12 | | | | |
| mDMD- | 44 | Mouse | TTTAGCATGTTCCCAGTTTT | 277 |
| E44g13 | | | | |
| mDMD- | 44 | Mouse | CTTAAGATACCATTTGTATT | 278 |
| E44g14 | | | | |
| mDMD- | 44 | Mouse | CTAAATACAAATGGTATCTT | 279 |
| E44g15 | | | | |
| mDMD- | 44 | Mouse | TACAAATGGTATCTTAAGgt | 280 |
| E44g16 | | | | |
| mDMD- | 44 | Mouse | AAATCTCAAAGTCTTACCTT | 281 |
| E44g17 | | | | |
| mDMD- | 44 | Mouse | ttatagGCGATTCGACAGAT | 282 |
| E44g18 | | | | |
| mDMD- | 44 | Mouse | catggatgaaataaggtaag | 283 |
| E44g19 | | | | |
| mDMD- | 44 | Mouse | ctgaaaaaatgaagccagca | 284 |
| E44g20 | | | | |
| mDMD- | 44 | Mouse | ATTTAATCAATGGCTGAATG | 285 |
| E44g21 | | | | |
| mDMD-E46g1 | 46 | Mouse | aattttgttattcttaatac | 286 |
| mDMD-E46g2 | 46 | Mouse | AAATGAATTTGTTTTGTGGC | 287 |
| mDMD-E46g3 | 46 | Mouse | AACATTGCTATTACTCCACT | 288 |
| mDMD-E46g4 | 46 | Mouse | AGCTGCTGCTCATCTCCAAG | 289 |
| mDMD-E46g5 | 46 | Mouse | AGAACAACTTGAACAAGTCA | 290 |
| mDMD-E46g6 | 46 | Mouse | gaattttgttattcttaata | 291 |
| mDMD-E46g7 | 46 | Mouse | TTGTTCTTCAATCctgtatt | 292 |
| mDMD-E46g8 | 46 | Mouse | ttattcttaatacagGATTG | 293 |
| mDMD-E46g9 | 46 | Mouse | aatacagGATTGAAGAACAA | 294 |
| mDMD- | 46 | Mouse | TTACTCCACTTGGAGATGAG | 295 |
| E46g10 | | | | |
| mDMD- | 46 | Mouse | GCTAAAAGAACAACTTGAAC | 296 |
| E46g11 | | | | |
| mDMD- | 46 | Mouse | gaaattacCTTGACTTGTTC | 297 |
| E46g12 | | | | |
| mDMD- | 46 | Mouse | AAGAACAACTTGAACAAGTC | 298 |
| E46g13 | | | | |
| mDMD- | 46 | Mouse | ACTTGTTCAAGTTGTTCTTT | 299 |
| E46g14 | | | | |
| mDMD- | 46 | Mouse | acacctctcagggatttagg | 300 |
| E46g15 | | | | |
| mDMD- | 46 | Mouse | ttcccttattaaaatcctca | 301 |
| E46g16 | | | | |
| mDMD- | 46 | Mouse | ctttatacaaataggccctg | 302 |
| E46g17 | | | | |
| mDMD-E51g4 | 51 | Mouse | TGAAATGATCATCAAACAGA | 303 |
| mDMD-E51g5 | 51 | Mouse | TCAATGAAATGATCATCAAA | 304 |
| mDMD-E51g6 | 51 | Mouse | ATGAAATGATCATCAAACAG | 305 |
| mDMD-E51g7 | 51 | Mouse | TGATCATCAAACAGAAGGTA | 306 |
| mDMD-E53g1 | 53 | Mouse | TGAAAGAATTCAGATTCAGT | 307 |
| mDMD-E53g2 | 53 | Mouse | AATTCAGATTCAGTGGGATG | 308 |
| mDMD-E53g3 | 53 | Mouse | TTCAAGAACAGCTGCAGAAC | 309 |
| mDMD-E53g4 | 53 | Mouse | ACAGTTGAATGAAATGTTAA | 310 |
| mDMD-E53g5 | 53 | Mouse | TGTTAAAGGATTCAACACAA | 311 |
| mDMD-E53g6 | 53 | Mouse | AAAGGATTCAACACAATGGC | 312 |
| mDMD-E53g7 | 53 | Mouse | AAAGAAGATCACAGAAACCA | 313 |
| mDMD-E53g8 | 53 | Mouse | TTGAAAGAATTCAGATTCAG | 314 |
| mDMD-E53g9 | 53 | Mouse | AGTGGGATGAGGTTCAAGAA | 315 |
| mDMD- | 53 | Mouse | AGCTGCAGAACAGGAGACAA | 316 |
| E53g10 | | | | |
| mDMD- | 53 | Mouse | TGAATCTGAATTCTTTCAAC | 317 |
| E53g11 | | | | |
| mDMD- | 53 | Mouse | CTTTCAACTGGAATAAAAAT | 318 |
| E53g12 | | | | |
| mDMD- | 53 | Mouse | CTTATTTTTATTCCAGTTGA | 319 |
| E53g13 | | | | |
| mDMD- | 53 | Mouse | TTATTCCAGTTGAAAGAATT | 320 |
| E53g14 | | | | |
| mDMD- | 53 | Mouse | CAGTTGAAAGAATTCAGATT | 321 |
| E53g15 | | | | |
| mDMD- | 53 | Mouse | GAATTCAGATTCAGTGGGAT | 322 |
| E53g16 | | | | |
| mDMD- | 53 | Mouse | GATTCAGTGGGATGAGGTTC | 323 |
| E53g17 | | | | |
| mDMD- | 53 | Mouse | ATGAGGTTCAAGAACAGCTG | 324 |
| E53g18 | | | | |
| mDMD- | 53 | Mouse | GTTCAAGAACAGCTGCAGAA | 325 |
| E53g19 | | | | |
| mDMD- | 53 | Mouse | AACTGTTGTCTCCTGTTCTG | 326 |
| E53g20 | | | | |
| mDMD- | 53 | Mouse | CAAGAACAGCTGCAGAACAG | 327 |
| E53g21 | | | | |
| mDMD- | 53 | Mouse | AAGATCACAGAAACCAAGGT | 328 |
| E53g22 | | | | |
| mDMD- | 53 | Mouse | CAGAAACCAAGGTTAGTGTC | 329 |
| E53g23 | | | | |

B. Scaffold

The scaffold sequence is the sequence within the gRNA that is responsible for nuclease (e.g., Cas9) binding. The scaffold sequence does not include the spacer/targeting sequence.

In some embodiments, the scaffold may be about 60 to about 70, about 70 to about 80, about 80 to about 90, about 90 to about 100, about 100 to about 110, about 110 to about 120, or about 120 to about 130 nucleotides in length. In some embodiments, the scaffold may be about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, about 100, about 101, about 102, about 103, about 104, about 105, about 106, about 107, about 108, about 109, about 110, about 111, about 112, about 113, about 114, about 115, about 116, about 117, about 118, about 119, about 120, about 121, about 122, about 123, about 124, or about 125 nucleotides in length. In some embodiments, the scaffold may be at least 60, at least 70, at least 80, at least 90, at least 100, at least 110, at least 120, or at least 125 nucleotides in length. In some embodiments, the scaffold may be 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, or 125 nucleotides in length.

In some embodiments, the scaffold may comprise a sequence of any one of SEQ ID NOs: 147-153 (shown in Table 4 below), or a sequence that is at least 90%, at least 95%, at least 96%, at least 97%, at least 98%, or at least 99% identical thereto.

TABLE 4
Exemplary scaffold sequences

| | SEQ ID |
|---|---|
| Sequence | NO: |
| GTTTAAGAGCTATGCTGGAAACAGCATAGCAAGTTTAAAT | 147 |
| AAGGCTAGTCCGTTATCAACTTGAAAAAGTGGCACCGAGT | |
| CGGTGCTTTTTTT | |
| GTTTTAGAGCTAGAAATAGCAGTTAAAATAAGGCTAGTCC | 148 |
| GTTATCAACTTGAAAAAGTGGCACCGAGTCGGTG | |
| GTTGGAACCATTCAAAACAGCATAGCAAGTTAAAATAAGG | 149 |
| CTAGTCCGTTATCAACTTGAAAAAGTGGCACCGAGTCGGT | |
| GCTTTTTT | |
| GTTTAAGAGCTATGAAACAGCATAGCAAGTTTAAATAAGG | 150 |
| CTAGTCCGTTATCAACTTGAAAAAGTGGCACCGAGTCGGT | |
| GCTTTTTTT | |
| GTTTAAGAGCTATGCGAAACAGCATAGCAAGTTTAAATAA | 151 |
| GGCTAGTCCGTTATCAACTTGAAAAAGTGGCACCGAGTCG | |
| GTGCTTTTTTT | |
| GTTTAAGAGCTATGCTGTTTGAAACAGCATAGCAAGTTTA | 152 |
| AATAAGGCTAGTCCGTTATCAACTTGAAAAAGTGGCACCG | |
| AGTCGGTGCTTTTTTT | |
| GTTTAAGAGCTATGCTGTTTTGGAAACAGCATAGCAAGTT | 153 |
| TAAATAAGGCTAGTCCGTTATCAACTTGAAAAAGTGGCAC | |
| CGAGTCGGTGCTTTTTTT | |

In some embodiments, a gRNA (spacer+scaffold) comprises a scaffold and a spacer as shown in Table 5 below, wherein “X” indicates that the particular combination is contemplated by the instant disclosure.

TABLE 5
Exemplary sgRNA (spacer + scaffold) sequences

| Spacer | Scaffold | Scaffold | Scaffold | Scaffold | Scaffold | Scaffold | Scaffold |
|---|---|---|---|---|---|---|---|
| Sequence | sequence | sequence | sequence | sequence | sequence | sequence | sequence |
| (SEQ | (SEQ ID | (SEQ ID | (SEQ ID | (SEQ ID | (SEQ ID | (SEQ ID | (SEQ ID |
| ID NO) | NO: 147) | NO: 148) | NO: 149) | NO: 150) | NO: 151) | NO: 152) | NO: 153) |
| 135 | X | X | X | X | X | X | X |
| 136 | X | X | X | X | X | X | X |
| 137 | X | X | X | X | X | X | X |
| 138 | X | X | X | X | X | X | X |
| 139 | X | X | X | X | X | X | X |
| 140 | X | X | X | X | X | X | X |
| 141 | X | X | X | X | X | X | X |
| 142 | X | X | X | X | X | X | X |
| 143 | X | X | X | X | X | X | X |
| 144 | X | X | X | X | X | X | X |
| 145 | X | X | X | X | X | X | X |
| 146 | X | X | X | X | X | X | X |
| 170 | X | X | X | X | X | X | X |
| 171 | X | X | X | X | X | X | X |
| 172 | X | X | X | X | X | X | X |
| 173 | X | X | X | X | X | X | X |
| 174 | X | X | X | X | X | X | X |
| 175 | X | X | X | X | X | X | X |
| 176 | X | X | X | X | X | X | X |
| 177 | X | X | X | X | X | X | X |
| 178 | X | X | X | X | X | X | X |
| 179 | X | X | X | X | X | X | X |
| 180 | X | X | X | X | X | X | X |
| 181 | X | X | X | X | X | X | X |
| 182 | X | X | X | X | X | X | X |
| 183 | X | X | X | X | X | X | X |
| 184 | X | X | X | X | X | X | X |
| 185 | X | X | X | X | X | X | X |
| 186 | X | X | X | X | X | X | X |
| 187 | X | X | X | X | X | X | X |
| 188 | X | X | X | X | X | X | X |
| 189 | X | X | X | X | X | X | X |
| 190 | X | X | X | X | X | X | X |
| 191 | X | X | X | X | X | X | X |
| 192 | X | X | X | X | X | X | X |
| 193 | X | X | X | X | X | X | X |
| 194 | X | X | X | X | X | X | X |
| 195 | X | X | X | X | X | X | X |
| 196 | X | X | X | X | X | X | X |
| 197 | X | X | X | X | X | X | X |
| 198 | X | X | X | X | X | X | X |
| 199 | X | X | X | X | X | X | X |
| 200 | X | X | X | X | X | X | X |
| 201 | X | X | X | X | X | X | X |
| 202 | X | X | X | X | X | X | X |
| 203 | X | X | X | X | X | X | X |
| 204 | X | X | X | X | X | X | X |
| 205 | X | X | X | X | X | X | X |
| 206 | X | X | X | X | X | X | X |
| 207 | X | X | X | X | X | X | X |
| 208 | X | X | X | X | X | X | X |
| 209 | X | X | X | X | X | X | X |
| 210 | X | X | X | X | X | X | X |
| 211 | X | X | X | X | X | X | X |
| 212 | X | X | X | X | X | X | X |
| 213 | X | X | X | X | X | X | X |
| 214 | X | X | X | X | X | X | X |
| 215 | X | X | X | X | X | X | X |
| 216 | X | X | X | X | X | X | X |
| 217 | X | X | X | X | X | X | X |
| 218 | X | X | X | X | X | X | X |
| 219 | X | X | X | X | X | X | X |
| 220 | X | X | X | X | X | X | X |
| 221 | X | X | X | X | X | X | X |
| 222 | X | X | X | X | X | X | X |
| 223 | X | X | X | X | X | X | X |
| 224 | X | X | X | X | X | X | X |
| 225 | X | X | X | X | X | X | X |
| 226 | X | X | X | X | X | X | X |
| 227 | X | X | X | X | X | X | X |
| 228 | X | X | X | X | X | X | X |
| 229 | X | X | X | X | X | X | X |
| 230 | X | X | X | X | X | X | X |
| 231 | X | X | X | X | X | X | X |
| 232 | X | X | X | X | X | X | X |
| 233 | X | X | X | X | X | X | X |
| 234 | X | X | X | X | X | X | X |
| 235 | X | X | X | X | X | X | X |
| 236 | X | X | X | X | X | X | X |
| 237 | X | X | X | X | X | X | X |
| 238 | X | X | X | X | X | X | X |
| 239 | X | X | X | X | X | X | X |
| 240 | X | X | X | X | X | X | X |
| 241 | X | X | X | X | X | X | X |
| 242 | X | X | X | X | X | X | X |
| 243 | X | X | X | X | X | X | X |
| 244 | X | X | X | X | X | X | X |
| 245 | X | X | X | X | X | X | X |
| 246 | X | X | X | X | X | X | X |
| 247 | X | X | X | X | X | X | X |
| 248 | X | X | X | X | X | X | X |
| 249 | X | X | X | X | X | X | X |
| 250 | X | X | X | X | X | X | X |
| 251 | X | X | X | X | X | X | X |
| 252 | X | X | X | X | X | X | X |
| 253 | X | X | X | X | X | X | X |
| 254 | X | X | X | X | X | X | X |
| 255 | X | X | X | X | X | X | X |
| 256 | X | X | X | X | X | X | X |
| 257 | X | X | X | X | X | X | X |
| 258 | X | X | X | X | X | X | X |
| 259 | X | X | X | X | X | X | X |
| 260 | X | X | X | X | X | X | X |

In some embodiments, the sgRNA has a sequence (spacer+scaffold) of any one of SEQ ID NO: 154 to 165 (shown in Table 6, below), or a sequence that is at least 90%, at least 95%, at least 96%, at least 97%, at least 98%, or at least 99% identical thereto.

TABLE 6
Exemplary sgRNA (spacer + scaffold) sequences

| | SEQ ID |
|---|---|
| sgRNA sequence (spacer + scaffold) | NO |
| AAAGAAAATCACAGAAACCAGTTTAAGAGCTATGCTGGAA | 154 |
| ACAGCATAGCAAGTTTAAATAAGGCTAGTCCGTTATCAAC | |
| TTGAAAAAGTGGCACCGAGTCGGTGCTTTTTTT | |
| AATTCAGAATCAGTGGGATGGTTTAAGAGCTATGCTGGAA | 155 |
| ACAGCATAGCAAGTTTAAATAAGGCTAGTCCGTTATCAAC | |
| TTGAAAAAGTGGCACCGAGTCGGTGCTTTTTTT | |
| TTGAAAGAATTCAGAATCAGGTTTAAGAGCTATGCTGGAA | 156 |
| ACAGCATAGCAAGTTTAAATAAGGCTAGTCCGTTATCAAC | |
| TTGAAAAAGTGGCACCGAGTCGGTGCTTTTTTT | |
| CAAGAACACCTTCAGAACCGGTTTAAGAGCTATGCTGGAA | 157 |
| ACAGCATAGCAAGTTTAAATAAGGCTAGTCCGTTATCAAC | |
| TTGAAAAAGTGGCACCGAGTCGGTGCTTTTTTT | |
| TATGTGTTACCTACCCTTGTGTTTAAGAGCTATGCTGGAA | 158 |
| ACAGCATAGCAAGTTTAAATAAGGCTAGTCCGTTATCAAC | |
| TTGAAAAAGTGGCACCGAGTCGGTGCTTTTTTT | |
| GTACAAGGACCGACAAGGGTGTTTAAGAGCTATGCTGGAA | 159 |
| ACAGCATAGCAAGTTTAAATAAGGCTAGTCCGTTATCAAC | |
| TTGAAAAAGTGGCACCGAGTCGGTGCTTTTTTT | |
| TAAATACAAATGGTATCTTAGTTTAAGAGCTATGCTGGAA | 160 |
| ACAGCATAGCAAGTTTAAATAAGGCTAGTCCGTTATCAAC | |
| TTGAAAAAGTGGCACCGAGTCGGTGCTTTTTTT | |
| GGGAACATGCTAAATACAAAGTTTAAGAGCTATGCTGGAA | 161 |
| ACAGCATAGCAAGTTTAAATAAGGCTAGTCCGTTATCAAC | |
| TTGAAAAAGTGGCACCGAGTCGGTGCTTTTTTT | |
| ACAAATCAAAGACTTACCTTGTTTAAGAGCTATGCTGGAA | 162 |
| ACAGCATAGCAAGTTTAAATAAGGCTAGTCCGTTATCAAC | |
| TTGAAAAAGTGGCACCGAGTCGGTGCTTTTTTT | |
| AATTTTATTCTTCTTTCTCCGTTTAAGAGCTATGCTGGAA | 163 |
| ACAGCATAGCAAGTTTAAATAAGGCTAGTCCGTTATCAAC | |
| TTGAAAAAGTGGCACCGAGTCGGTGCTTTTTTT | |
| AGAAAAGCTTGAGCAAGTCAGTTTAAGAGCTATGCTGGAA | 164 |
| ACAGCATAGCAAGTTTAAATAAGGCTAGTCCGTTATCAAC | |
| TTGAAAAAGTGGCACCGAGTCGGTGCTTTTTTT | |
| TGTATGCTTTTCTGTTAAAGGTTTAAGAGCTATGCTGGAA | 165 |
| ACAGCATAGCAAGTTTAAATAAGGCTAGTCCGTTATCAAC | |
| TTGAAAAAGTGGCACCGAGTCGGTGCTTTTTTT | |

In some embodiments, a nucleic acid comprises one copy of the sequence encoding the sgRNA. In some embodiments, a nucleic acid comprises two, three, four, or five copies of the sequence encoding the sgRNA.

In some embodiments, a nucleic acid comprises a sequence encoding a promoter, wherein the promoter drives expression of the sgRNA. In some embodiments, the nucleic acid comprises two copies of the sequence encoding a sgRNA, wherein expression of the first copy of the sgRNA is driven by a first promoter, and expression of the second copy of the sgRNA is driven by a second promoter.

In some embodiments, the nucleic acid comprises three copies of the sequence encoding a sgRNA, wherein expression of the first copy of the sgRNA is driven by a first promoter, expression of the second copy of the sgRNA is driven by a second promoter, and expression of the third copy of the sgRNA is driven by a third promoter.

In some embodiments, the nucleic acid comprises four copies of the sequence encoding a sgRNA, wherein expression of the first copy of the sgRNA is driven by a first promoter, expression of the second copy of the sgRNA is driven by a second promoter, expression of the third copy of the sgRNA is driven by a third promoter, and expression of the fourth copy of the sgRNA is driven by a fourth promoter.

In some embodiments, the nucleic acid comprises five copies of the sequence encoding a sgRNA, wherein expression of the first copy of the sgRNA is driven by a first promoter, expression of the second copy of the sgRNA is driven by a second promoter, expression of the third copy of the sgRNA is driven by a third promoter, expression of the fourth copy of the sgRNA is driven by a fourth promoter, and expression of the fifth copy of the sgRNA is driven by a fifth promoter.

In some embodiments, a nucleic acid sequence comprising a sequence encoding a sgRNA further comprises a sequence encoding a nuclease. The nuclease may be, for example, a Type II, Type V-A, Type V-B, Type V-C, Type V-U, or Type VI-B nuclease. Exemplary nucleases include, but are not limited to a TALEN, a meganuclease, a zinc-finger nuclease, or a Cas9, Cas12a, Cas12b, Cas12c, Tnp-B like, Cas13a (C2c2), Cas13b, or Cas14 nuclease.

In some embodiments, the nuclease is a Cas9 nuclease. In some embodiments, the Cas9 nuclease is a Streptococcus pyogenes or Streptococcus aureus Cas9. In some embodiments, the nuclease is a modified Cas9 nuclease. In some embodiments, the nuclease is a modified Streptococcus pyogenes Cas9 or a modified Streptococcus aureus Cas9.

CRISPRs (clustered regularly interspaced short palindromic repeats) are DNA loci containing short repetitions of base sequences. Each repetition is followed by short segments of “spacer DNA” from previous exposures to a virus. CRISPRs are found in approximately 40% of sequenced eubacteria genomes and 90% of sequenced archaea. CRISPRs are often associated with Cas genes that code for proteins related to CRISPRs. The CRISPR/Cas system is a prokaryotic immune system that confers resistance to foreign genetic elements such as plasmids and phages and provides a form of acquired immunity. CRISPR spacers recognize and silence these exogenous genetic elements like RNAi in eukaryotic organisms.

CRISPR repeats can range in size from 24 to 48 base pairs. They usually show some dyad symmetry, implying the formation of a secondary structure such as a hairpin, but are not truly palindromic. Repeats are separated by spacers of similar length.

CRISPR-associated (cas) genes are often associated with CRISPR repeat-spacer arrays. More than forty different Cas protein families have been described. Of these protein families, Cast appears to be ubiquitous among different CRISPR/Cas systems. Particular combinations of cas genes and repeat structures have been used to define 8 CRISPR subtypes (E. coli, Ypest, Nmeni, Dvulg, Tneap, Hmari, Apern, and Mtube), some of which are associated with an additional gene module encoding repeat-associated mysterious proteins (RAMPs). More than one CRISPR subtype may occur in a single genome. The sporadic distribution of the CRISPR/Cas subtypes suggests that the system is subject to horizontal gene transfer during microbial evolution.

Exogenous DNA is processed by proteins encoded by Cas genes into small elements (˜30 base pairs in length), which are then inserted into the CRISPR locus near the leader sequence. RNAs from the CRISPR loci are constitutively expressed and are processed by Cas proteins to small RNAs composed of individual, exogenously-derived sequence elements with a flanking repeat sequence. The RNAs guide other Cas proteins to silence exogenous genetic elements at the RNA or DNA level. Evidence suggests functional diversity among CRISPR subtypes. The Cse (Cas subtype E. coli) proteins (called CasA-E in E. coli) form a functional complex, Cascade, that processes CRISPR RNA transcripts into spacer-repeat units that Cascade retains. In other prokaryotes, Cas6 processes the CRISPR transcripts. Interestingly, CRISPR-based phage inactivation in E. coli requires Cascade and Cas3, but not Cas1 and Cas2. The Cmr (Cas RAMP module) proteins found in Pyrococcus furiosus and other prokaryotes form a functional complex with small CRISPR RNAs that recognizes and cleaves complementary target RNAs. RNA-guided CRISPR enzymes are classified as type V restriction enzymes.

Cas9 is a nuclease, an enzyme specialized for cutting DNA, with two active cutting sites, one for each strand of the double helix. One or both sites may be inactivated while preserving Cas9's ability to locate its target DNA. tracrRNA (i.e., a scaffold sequence) and spacer RNA may be combined into a “single-guide RNA” molecule that, mixed with Cas9, could find and cut the correct DNA targets. Such synthetic guide RNAs can be used for gene editing.

Cas9 proteins are highly enriched in pathogenic and commensal bacteria. CRISPR/Cas-mediated gene regulation may contribute to the regulation of endogenous bacterial genes, particularly during bacterial interaction with eukaryotic hosts. For example, Cas protein Cas9 of Francisella novicida uses a unique, small, CRISPR/Cas-associated RNA (scaRNA) to repress an endogenous transcript encoding a bacterial lipoprotein that is critical for F. novicida to dampen host response and promote virulence. Delivery of Cas9 DNA sequences also is contemplated.

Clustered Regularly Interspaced Short Palindromic Repeats from Prevotella and Francisella 1 or CRISPR/Cpf1 is a DNA-editing technology analogous to the CRISPR/Cas9 system. Cpf1 is an RNA-guided endonuclease of a class II CRISPR/Cas system. This acquired immune mechanism is found in Prevotella and Francisella bacteria. It prevents genetic damage from viruses. Cpf1 genes are associated with the CRISPR locus, coding for an endonuclease that use a guide RNA to find and cleave viral DNA. Cpf1 is a smaller and simpler endonuclease than Cas9, overcoming some of the CRISPR/Cas9 system limitations. CRISPR/Cpf1 has multiple applications, including treatment of genetic illnesses and degenerative conditions.

Cpf1 appears in many bacterial species. The Two Cpf1 enzymes from Acidaminococcus and Lachnospiraceae display efficient genome-editing activity in human cells.

A smaller version of Cas9 from the bacterium Staphylococcus aureus is a potential alternative to Cpf1.

The systems CRISPR/Cas are separated into three classes. Class 1 uses several Cas proteins together with the CRISPR RNAs (crRNA) to build a functional endonuclease. Class 2 CRISPR systems use a single Cas protein with a crRNA. Cpf1 has been recently identified as a Class II, Type V CRISPR/Cas systems containing a 1,300 amino acid protein.

The Cpf1 locus contains a mixed alpha/beta domain, a RuvC-I followed by a helical region, a RuvC-II and a zinc finger-like domain. The Cpf1 protein has a RuvC-like endonuclease domain that is similar to the RuvC domain of Cas9. Furthermore, Cpf1 does not have a HNH endonuclease domain, and the N-terminal of Cpf1 does not have the alfa-helical recognition lobe of Cas9.

Cpf1 CRISPR-Cas domain architecture shows that Cpf1 is functionally unique, being classified as Class 2, type V CRISPR system. The Cpf1 loci encode Cas1, Cas2 and Cas4 proteins more similar to types I and III than from type II systems. Database searches suggest the abundance of Cpf1-family proteins in many bacterial species.

Functional Cpf1 doesn't need the tracrRNA, therefore, only crRNA is required. This benefits genome editing because Cpf1 is not only smaller than Cas9, but also it has a smaller sgRNA molecule (proximately half as many nucleotides as Cas9).

The Cpf1-crRNA complex cleaves target DNA or RNA by identification of a protospacer adjacent motif 5′-YTN-3′ (where “Y” is a pyrimidine and “N” is any nucleobase) or 5′-TTN-3′, in contrast to the G-rich PAM targeted by Cas9. After identification of PAM, Cpf1 introduces a sticky-end-like DNA double-stranded break of 4 or 5 nucleotides overhang.

The CRISPR/Cpf1 system consist of a Cpf1 enzyme and a guide RNA that finds and positions the complex at the correct spot on the double helix to cleave target DNA. CRISPR/Cpf1 systems activity has three stages:
Adaptation, during which Cas1 and Cas2 proteins facilitate the adaptation of small fragments of DNA into the CRISPR array;
Formation of crRNAs: processing of pre-cr-RNAs producing of mature crRNAs to guide the Cas protein; and
Interference, in which the Cpf1 is bound to a crRNA to form a binary complex to identify and cleave a target DNA sequence.

### II. NUCLEIC ACID DELIVERY

As discussed above, in certain embodiments, expression cassettes are employed to express a transcription factor product, either for subsequent purification and delivery to a cell/subject, or for use directly in a genetic-based delivery approach. Expression requires that appropriate signals be provided in the vectors, and include various regulatory elements such as enhancers/promoters from both viral and mammalian sources that drive expression of the genes of interest in cells. Elements designed to optimize messenger RNA stability and translatability in host cells also are defined. The conditions for the use of a number of dominant drug selection markers for establishing permanent, stable cell clones expressing the products are also provided, as is an element that links expression of the drug selection markers to expression of the polypeptide.

A. Regulatory Elements

Throughout this application, the term “expression cassette” is meant to include any type of genetic construct containing a nucleic acid coding for a gene product in which part or all of the nucleic acid encoding sequence is capable of being transcribed and translated, i.e., is under the control of a promoter. A “promoter” refers to a DNA sequence recognized by the synthetic machinery of the cell, or introduced synthetic machinery, responsible for initiating the specific transcription of a gene. The phrase “under transcriptional control” means that the promoter is in the correct location and orientation in relation to the nucleic acid to control RNA polymerase initiation and expression of the gene. An “expression vector” is meant to include expression cassettes comprised in a genetic construct that is capable of replication, and thus including one or more of origins of replication, transcription termination signals, poly-A regions, selectable markers, and multipurpose cloning sites.

The term promoter will be used here to refer to a group of transcriptional control modules that are clustered around the initiation site for RNA polymerase II. Much of the thinking about how promoters are organized derives from analyses of several viral promoters, including those for the HSV thymidine kinase (tk) and SV40 early transcription units. These studies, augmented by more recent work, have shown that promoters are composed of discrete functional modules, each consisting of approximately 7-20 bp of DNA, and containing one or more recognition sites for transcriptional activator or repressor proteins.

At least one module in each promoter functions to position the start site for RNA synthesis. The best-known example of this is the TATA box, but in some promoters lacking a TATA box, such as the promoter for the mammalian terminal deoxynucleotidyl transferase gene and the promoter for the SV40 late genes, a discrete element overlying the start site itself helps to fix the place of initiation.

In some embodiments, the sgRNA, Cas9 or Cpf1 constructs of the disclosure are expressed by a muscle-cell specific promoter. This muscle-cell specific promoter may be constitutively active or may be an inducible promoter.

Additional promoter elements regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either co-operatively or independently to activate transcription.

In certain embodiments, viral promoters such as the human cytomegalovirus (CMV) immediate early gene promoter, the SV40 early promoter, the Rous sarcoma virus long terminal repeat, rat insulin promoter and glyceraldehyde-3-phosphate dehydrogenase can be used to obtain high-level expression of the coding sequence of interest. The use of other viral or mammalian cellular or bacterial phage promoters which are well-known in the art to achieve expression of a coding sequence of interest is contemplated as well, provided that the levels of expression are sufficient for a given purpose. By employing a promoter with well-known properties, the level and pattern of expression of the protein of interest following transfection or transformation can be optimized. Further, selection of a promoter that is regulated in response to specific physiologic signals can permit inducible expression of the gene product.

Enhancers are genetic elements that increase transcription from a promoter located at a distant position on the same molecule of DNA. Enhancers are organized much like promoters. That is, they are composed of many individual elements, each of which binds to one or more transcriptional proteins. The basic distinction between enhancers and promoters is operational. An enhancer region as a whole is able to stimulate transcription at a distance; this need not be true of a promoter region or its component elements. On the other hand, a promoter has one or more elements that direct initiation of RNA synthesis at a particular site and in a particular orientation, whereas enhancers lack these specificities. Promoters and enhancers are often overlapping and contiguous, often seeming to have a very similar modular organization.

Below is a list of promoters/enhancers and inducible promoters/enhancers that could be used to drive expression of a nucleic acid encoding a gene of interest in an expression construct. Additionally, any promoter/enhancer combination (as per the Eukaryotic Promoter Data Base EPDB) could also be used to drive expression of the gene. Eukaryotic cells can support cytoplasmic transcription from certain bacterial promoters if the appropriate bacterial polymerase is provided, either as part of the delivery complex or as an additional genetic expression construct.

The promoter and/or enhancer may be, for example, immunoglobulin light chain, immunoglobulin heavy chain, T-cell receptor, HLA DQ a and/or DQ β, β-interferon, interleukin-2, interleukin-2 receptor, MHC class II 5, MHC class II HLA-Dra, β-Actin, muscle creatine kinase (MCK), prealbumin (transthyretin), elastase I, metallothionein collagenase, albumin, α-fetoprotein, t-globin, β-fos, c-HA-ras, insulin, neural cell adhesion molecule (NCAM), α₁-antitrypain, H2B (TH2B) histone, mouse and/or type I collagen, glucose-regulated proteins (GRP94 and GRP78), rat growth hormone, human serum amyloid A (SAA), troponin I (TN I), platelet-derived growth factor (PDGF), duchenne muscular dystrophy, SV40, polyoma, retroviruses, papilloma virus, hepatitis B virus, human immunodeficiency virus, cytomegalovirus (CMV), and gibbon ape leukemia virus.

In some embodiments, inducible elements may be used. In some embodiments, the inducible element is, for example, MTII, MMTV (mouse mammary tumor virus), β-interferon, adenovirus 5 E2, collagenase, stromelysin, SV40, murine MX gene, GRP78 gene, α-2-macroglobulin, vimentin, MHC class I gene H-2κb, HSP70, proliferin, tumor necrosis factor, and/or thyroid stimulating hormone a gene. In some embodiments, the inducer is phorbol ester (TFA), heavy metals, glucocorticoids, poly(rDx, poly(rc), ElA, phorbol ester (TPA), interferon, Newcastle Disease Virus, A23187, IL-6, serum, interferon, SV40 large T antigen, PMA, and/or thyroid hormone. Any of the inducible elements described herein may be used with any of the inducers described herein.

Of particular interest are muscle specific promoters. These include the myosin light chain-2 promoter, the α-actin promoter, the troponin 1 promoter; the Na⁺/Ca²⁺ exchanger promoter, the dystrophin promoter, the α7 integrin promoter, the brain natriuretic peptide promoter and the αB-crystallin/small heat shock protein promoter, α-myosin heavy chain promoter and the ANF promoter.

In some embodiments, the muscle specific promoter is the CK8 promoter. The CK8 promoter has the following sequence (SEQ ID NO: 331):

| | |
|---|---|
| 1 | CTAGACTAGC ATGCTGCCCA TGTAAGGAGG CAAGGCCTGG |
| | GGACACCCGA GATGCCTGGT |
| 61 | TATAATTAAC CCAGACATGT GGCTGCCCCC CCCCCCCCAA |
| | CACCTGCTGC CTCTAAAAAT |
| 121 | AACCCTGCAT GCCATGTTCC CGGCGAAGGG CCAGCTGTCC |
| | CCCGCCAGCT AGACTCAGCA |
| 181 | CTTAGTTTAG GAACCAGTGA GCAAGTCAGC CCTTGGGGCA |
| | GCCCATACAA GGCCATGGGG |
| 241 | CTGGGCAAGC TGCACGCCTG GGTCCGGGGT GGGCACGGTG |
| | CCCGGGCAAC GAGCTGAAAG |
| 301 | CTCATCTGCT CTCAGGGGCC CCTCCCTGGG GACAGCCCCT |
| | CCTGGCTAGT CACACCCTGT |
| 361 | AGGCTCCTCT ATATAACCCA GGGGCACAGG GGCTGCCCTC |
| | ATTCTACCAC CACCTCCACA |
| 421 | GCACAGACAG ACACTCAGGA GCCAGCCAGC. |

In some embodiments, the muscle-cell cell specific promoter is a variant of the CK8 promoter, called CK8e. The CK8e promoter has the following sequence (SEQ ID NO. 332):

| | |
|---|---|
| 1 | TGCCCATGTA AGGAGGCAAG GCCTGGGGAC ACCCGAGATG |
| | CCTGGTTATA ATTAACCCAG |
| 61 | ACATGTGGCT GCCCCCCCCC CCCCAACACC TGCTGCCTCT |
| | AAAAATAACC CTGCATGCCA |
| 121 | TGTTCCCGGC GAAGGGCCAG CTGTCCCCCG CCAGCTAGAC |
| | TCAGCACTTA GTTTAGGAAC |
| 181 | CAGTGAGCAA GTCAGCCCTT GGGGCAGCCC ATACAAGGCC |
| | ATGGGGCTGG GCAAGCTGCA |
| 241 | CGCCTGGGTC CGGGGTGGGC ACGGTGCCCG GGCAACGAGC |
| | TGAAAGCTCA TCTGCTCTCA |
| 301 | GGGGCCCCTC CCTGGGGACA GCCCCTCCTG GCTAGTCACA |
| | CCCTGTAGGC TCCTCTATAT |
| 361 | AACCCAGGGG CACAGGGGCT GCCCTCATTC TACCACCACC |
| | TCCACAGCAC AGACAGACAC |
| 421 | TCAGGAGCCA GCCAGC. |

Where a cDNA insert is employed, one will typically desire to include a polyadenylation signal to effect proper polyadenylation of the gene transcript. Any polyadenylation sequence may be employed such as human growth hormone and SV40 polyadenylation signals. Also contemplated as an element of the expression cassette is a terminator. These elements can serve to enhance message levels and to minimize read through from the cassette into other sequences.

B. Self-Cleaving Peptides

In some embodiments, the nucleic acids and/or expression constructs disclosed herein may encode a self-cleaving peptide.

In some embodiments of self-cleaving peptides of the disclosure, the self-cleaving peptide is a 2A peptide. In some embodiments, a 2A-like self-cleaving domain from the insect virus Thosea asigna (TaV 2A peptide) (SEQ ID NO: 333, EGRGSLLTCGDVEENPGP) is used. These 2A-like domains have been shown to function across eukaryotes and cause cleavage of amino acids to occur co-translationally within the 2A-like peptide domain. Therefore, inclusion of TaV 2A peptide allows the expression of multiple proteins from a single mRNA transcript. Importantly, the domain of TaV when tested in eukaryotic systems has shown greater than 99% cleavage activity. Other acceptable 2A-like peptides include, but are not limited to, equine rhinitis A virus (ERAV) 2A peptide (SEQ ID NO: 334; QCTNYALLKLAGDVESNPGP), porcine teschovirus-1 (PTV1) 2A peptide (SEQ ID NO: 335; ATNFSLLKQAGDVEENPGP) and foot and mouth disease virus (FMDV) 2A peptide (SEQ ID NO: 336; PVKQLLNFDLLKLAGDVESNPGP) or modified versions thereof.

In some embodiments, the 2A peptide is used to express a reporter and a Cas9 or a Cpf1 simultaneously. The reporter may be, for example, GFP.

Other self-cleaving peptides that may be used include, but are not limited to nuclear inclusion protein a (Nia) protease, a P1 protease, a 3C protease, an L protease, a 3C-like protease, or modified versions thereof.

C. Delivery of Expression Vectors

There are a number of other ways in which expression vectors may introduced into cells. In certain embodiments, the expression construct comprises a virus or engineered construct derived from a viral genome. The ability of certain viruses to enter cells via receptor-mediated endocytosis, to integrate into host cell genome and express viral genes stably and efficiently have made them attractive candidates for the transfer of foreign genes into mammalian cells. In some embodiments, the gene editing compositions described herein are administered to a cell or to a subject using a non-viral vector or a viral vector. In some embodiments, the gene editing compositions described herein are administered to a cell or to a subject using a recombinant vector (e.g., a recombinant viral or a recombinant non-viral vector). In some embodiments, a recombinant vector comprises a nucleic acid of the disclosure, i.e., a nucleic acid comprising a sequence encoding a single guide RNA (sgRNA) comprising a spacer sequence and a scaffold sequence, wherein the spacer sequence targets an exon sequence of the DMD gene, such as a sequence of exon 43, 44, 46, 50, or 53. In some embodiments, the recombinant vector is a plasmid. In some embodiments, the recombinant vector is an expression vector.

Exemplary non-viral vectors for use with the compositions and methods described herein comprise nanoparticles (e.g., polymeric nanoparticles), liposomes (e.g., cationic liposomes), naked DNA, cationic lipid-DNA complexes, lipid emulsions, calcium phosphate, polymer complexes, or combinations thereof.

Exemplary viral vectors for use with the compositions and methods described herein include vectors based on adeno-associated virus (AAV), adenovirus, lentivirus, retrovirus, or a hybrid virus. In some embodiments, the viral vectors of the instant disclosure are replication defective, or at least conditionally replication defective.

The AAV genome may be from any naturally derived serotype or isolate or clade of AAV. Thus, the AAV genome may be the full genome of a naturally occurring AAV virus. As is known to the skilled person, AAV viruses occurring in nature may be classified according to various biological systems.

Commonly, AAV viruses are referred to in terms of their serotype. A serotype corresponds to a variant subspecies of AAV which owing to its profile of expression of capsid surface antigens has a distinctive reactivity which can be used to distinguish it from other variant subspecies. Typically, a virus having a particular AAV serotype does not efficiently cross-react with neutralizing antibodies specific for any other AAV serotype. AAV serotypes include AAV1, AAV2, AAV3, AAV4, AAV5, AAV6, AAV7, AAV8, AAV9, AAV10 and AAV11, also recombinant serotypes, such as Rec2 and Rec3, recently identified from primate brain. The sequences of AAV genomes or of elements of AAV genomes including ITR sequences, rep or cap genes for use methods and compositions described herein may be derived from the following accession numbers for AAV whole genome sequences: Adeno-associated virus 1 NC_002077, AF063497; Adeno-associated virus 2 NC_001401; Adeno-associated virus 3 NC_001729; Adeno-associated virus 3B NC_001863; Adeno-associated virus 4 NC_001829; Adeno-associated virus 5 Y18065, AF085716; Adeno-associated virus 6 NC_001862; Avian AAV ATCC VR-865 AY186198, AY629583, NC_004828; Avian AAV strain DA-1 NC_006263, AY629583; Bovine AAV NC_005889, AY388617.

AAV viruses may also be referred to in terms of clades or clones. This refers to the phylogenetic relationship of naturally derived AAV viruses, and typically to a phylogenetic group of AAV viruses which can be traced back to a common ancestor, and includes all descendants thereof. Additionally, AAV viruses may be referred to in terms of a specific isolate, i.e., a genetic isolate of a specific AAV virus found in nature. The term genetic isolate describes a population of AAV viruses which has undergone limited genetic mixing with other naturally occurring AAV viruses, thereby defining a recognizably distinct population at a genetic level.

In some embodiments, the gene editing compositions of the instant disclosure are delivered to a cell or to a patient using one or more AAV vectors. An AAV vector typically comprises an AAV expression cassette encapsidated by an AAV capsid protein. The serotype of the AAV vector may be selected from AAV1, AAV2, AAV3, AAV4, AAV5, AAV6, AAV7, AAV8, AAV9, AAV10, AAV11, AAV12, AAVRh74, AAV2i8, AAVRh10, AAV39, AAV43, AAVRh8, avian AAV, bovine AAV, canine AAV, equine AAV, and ovine AAV. In some embodiments, the AAV vector may be replication-defective or conditionally replication defective.

In some embodiments, the AAV vector is selected from any of the AAV vectors disclosed in Table 1 of WO 2019/028306, which is incorporated by reference herein in its entirety. In some embodiments, the AAV vector is selected from one of the serotypes listed in Table 7.

TABLE 7
AAV Serotypes

| | Seq |
|---|---|
| Serotype | ID No. |
| VOY101 | 1001 |
| VOY201 | 2260 |
| PHP.N/PHP.B-DGT | 1002 |
| AAVPHP.B or G2B-26 | 1003 |
| AAVPHP.B | 1004 |
| AAVG2B-13 | 1005 |
| AAVTH1.1-32 | 1006 |
| AAVTH1.1-35 | 1007 |
| PHP.S/G2Al2 | 1008 |
| AAV9/hu.14 K449R | 1009 |
| AAV1 | 1010 |
| AAV1 | 1011 |
| AAV1 | 1012 |
| AAV1.3 | 1013 |
| AAV1O | 1014 |
| AAV1O | 1015 |
| AAV1O | 1016 |
| AAV11 | 1017 |
| AAV12 | 1018 |
| AAV2 | 1019 |
| AAV2 | 1020 |
| AAV2 | 1021 |
| AAV2 | 1022 |
| AAV2 | 1023 |
| AAV2.5T | 1024 |
| AAV223.10 | 1025 |
| AAV223.2 | 1026 |
| AAV223.2 | 1027 |
| AAV223.4 | 1028 |
| AAV223.4 | 1029 |
| AAV223.5 | 1030 |
| AAV223.5 | 1031 |
| AAV223.6 | 1032 |
| AAV223.6 | 1033 |
| AAV223.7 | 1034 |
| AAV223.7 | 1035 |
| AAV29.3 | 1036 |
| AAV29.4 | 1037 |
| AAV29.5 | 1038 |
| AAV29.5 (AAVbb.2) | 1039 |
| AAV3 | 1040 |
| AAV3 | 1041 |
| AAV3 | 1042 |
| AAV3.3b | 1043 |
| AAV3-3 | 1044 |
| AAV3-3 | 1045 |
| AAV3a | 1046 |
| AAV3a | 1047 |
| AAV3b | 1048 |
| AAV3b | 1049 |
| AAV3b | 1050 |
| AAV4 | 1051 |
| AAV4 | 1052 |
| AAV4 | 1053 |
| AAV4 | 1054 |
| AAV4 | 1055 |
| AAV4 | 1056 |
| AAV4 | 1057 |
| AAV4 | 1058 |
| AAV4 | 1059 |
| AAV4 | 1060 |
| AAV4 | 1061 |
| AAV4 | 1062 |
| AAV4 | 1063 |
| AAV4 | 1064 |
| AAV4 | 1065 |
| AAV4 | 1066 |
| AAV4 | 1067 |
| AAV4 | 1068 |
| AAV4 | 1069 |
| AAV4 | 1070 |
| AAV42.2 | 1071 |
| AAV42.2 | 1072 |
| AAV42.3b | 1073 |
| AAV42.3B | 1074 |
| AAV42.4 | 1075 |
| AAV42.4 | 1076 |
| AAV42.8 | 1077 |
| AAV42.8 | 1078 |
| AAV43.1 | 1079 |
| AAV43.1 | 1080 |
| AAV43.12 | 1081 |
| AAV43.12 | 1082 |
| AAV43.20 | 1083 |
| AAV43.20 | 1084 |
| AAV43.21 | 1085 |
| AAV43.21 | 1086 |
| AAV43.23 | 1087 |
| AAV43.23 | 1088 |
| AAV43.25 | 1089 |
| AAV43.25 | 1090 |
| AAV43.5 | 1091 |
| AAV43.5 | 1092 |
| AAV4-4 | 1093 |
| AAV4-4 | 1094 |
| AAV44.1 | 1095 |
| AAV44.1 | 1096 |
| AAV44.5 | 1097 |
| AAV44.5 | 1098 |
| AAV4407 | 1099 |
| AAV5 | 1100 |
| AAV5 | 1101 |
| AAV5 | 1102 |
| AAV5 | 1103 |
| AAV6 | 1104 |
| AAV6 | 1105 |
| AAV6 | 1106 |
| AAV6 | 1107 |
| AAV6 | 1108 |
| AAV6 | 1109 |
| AAV6.1 | 1110 |
| AAV6.12 | 1111 |
| AAV6.2 | 1112 |
| AAV7 | 1113 |
| AAV7 | 1114 |
| AAV7 | 1115 |
| AAV7 | 1116 |
| AAV7 | 1117 |
| AAV7 | 1118 |
| AAV7 | 1119 |
| AAV8 | 1120 |
| AAV8 | 1121 |
| AAV8 | 1122 |
| AAV8 | 1123 |
| AAV8 | 1124 |
| AAV8 | 1125 |
| AAV-8b | 1126 |
| AAV-8b | 1127 |
| AAV-8h | 1128 |
| AAV-8h | 1129 |
| AAV9 | 1130 |
| AAV9 | 1131 |
| AAV9 | 1132 |
| AAV9 | 1133 |
| AAV9 | 1134 |
| AAV9 (AAVhu.14) | 1135 |
| AAV9 (AAVhu.14) | 1136 |
| AAVA3.1 | 1137 |
| AAVA3.3 | 1138 |
| AAVA3.3 | 1139 |
| AAVA3.4 | 1140 |
| AAVA3.4 | 1141 |
| AAVA3.5 | 1142 |
| AAVA3.5 | 1143 |
| AAVA3.7 | 1144 |
| AAVA3.7 | 1145 |
| AAV29.3 (AAVbb.1) | 1146 |
| AAVC2 | 1147 |
| AAVCh.5 | 1148 |
| AAVcy.2 (AAV13.3) | 1149 |
| AAV24.1 | 1150 |
| AAVcy.3 (AAV24.1) | 1151 |
| AAV27.3 | 1152 |
| AAVcy.4 (AAV27.3) | 1153 |
| AAVcy.5 | 1154 |
| AAV7.2 | 1155 |
| AAVcy.5 (AAV7.2) | 1156 |
| AAV16.3 | 1157 |
| AAVcy.6 (AAV16.3) | 1158 |
| AAVcy.5 | 1159 |
| AAVcy.5 | 1160 |
| AAVCy.5R1 | 1161 |
| AAVCy.5R2 | 1162 |
| AAVCy.5R3 | 1163 |
| AAVCy.5R4 | 1164 |
| AAVDJ | 1165 |
| AAVDJ | 1166 |
| AAVDJ-8 | 1167 |
| AAVDJ-8 | 1168 |
| AAVF5 | 1169 |
| AAVH2 | 1170 |
| AAVH6 | 1171 |
| AAVhE1.1 | 1172 |
| AAVhEr1.14 | 1173 |
| AAVhEr1.16 | 1174 |
| AAVhEr1.18 | 1175 |
| AAVhEr1.23 (AAVhEr2.29) | 1176 |
| AAVhEr1.35 | 1177 |
| AAVhEr1.36 | 1178 |
| AAVhEr1.5 | 1179 |
| AAVhEr1.7 | 1180 |
| AAVhEr1.8 | 1181 |
| AAVhEr2.16 | 1182 |
| AAVhEr2.30 | 1183 |
| AAVhEr2.31 | 1184 |
| AAVhEr2.36 | 1185 |
| AAVhEr2.4 | 1186 |
| AAVhEr3.1 | 1187 |
| AAVhu.1 | 1188 |
| AAVhu.1 | 1189 |
| AAVhu.10 (AAV16.8) | 1190 |
| AAVhu.10 (AAV16.8) | 1191 |
| AAVhu.11 (AAV16.12) | 1192 |
| AAVhu.11 (AAV16.12) | 1193 |
| AAVhu.12 | 1194 |
| AAVhu.12 | 1195 |
| AAVhu.13 | 1196 |
| AAVhu.13 | 1197 |
| AAVhu.136.1 | 1198 |
| AAVhu.140.1 | 1199 |
| AAVhu.140.2 | 1200 |
| AAVhu.145.6 | 1201 |
| AAVhu.15 | 1202 |
| AAVhu.15 (AAV33.4) | 1203 |
| AAVhu.156.1 | 1204 |
| AAVhu.16 | 1205 |
| AAVhu.16 (AAV33.8) | 1206 |
| AAVhu.17 | 1207 |
| AAVhu.17 (AAV33.12) | 1208 |
| AAVhu.172.1 | 1209 |
| AAVhu.172.2 | 1210 |
| AAVhu.173.4 | 1211 |
| AAVhu.173.8 | 1212 |
| AAVhu.18 | 1213 |
| AAVhu.18 | 1214 |
| AAVhu.19 | 1215 |
| AAVhu.19 | 1216 |
| AAVhu.2 | 1217 |
| AAVhu.2 | 1218 |
| AAVhu.20 | 1219 |
| AAVhu.20 | 1220 |
| AAVhu.21 | 1221 |
| AAVhu.21 | 1222 |
| AAVhu.22 | 1223 |
| AAVhu.22 | 1224 |
| AAVhu.23 | 1225 |
| AAVhu.23.2 | 1226 |
| AAVhu.24 | 1227 |
| AAVhu.24 | 1228 |
| AAVhu.25 | 1229 |
| AAVhu.25 | 1230 |
| AAVhu.26 | 1231 |
| AAVhu.26 | 1232 |
| AAVhu.27 | 1233 |
| AAVhu.27 | 1234 |
| AAVhu.28 | 1235 |
| AAVhu.28 | 1236 |
| AAVhu.29 | 1237 |
| AAVhu.29 | 1238 |
| AAVhu.29 | 1239 |
| AAVhu.29R | 1240 |
| AAVhu.3 | 1241 |
| AAVhu.3 | 1242 |
| AAVhu.30 | 1243 |
| AAVhu.30 | 1244 |
| AAVhu.31 | 1245 |
| AAVhu.31 | 1246 |
| AAVhu.32 | 1247 |
| AAVhu.32 | 1248 |
| AAVhu.33 | 1249 |
| AAVhu.33 | 1250 |
| AAVhu.34 | 1251 |
| AAVhu.34 | 1252 |
| AAVhu.35 | 1253 |
| AAVhu.35 | 1254 |
| AAVhu.36 | 1255 |
| AAVhu.36 | 1256 |
| AAVhu.37 | 1257 |
| AAVhu.37 (AAV106.1) | 1258 |
| AAVhu.38 | 1259 |
| AAVhu.39 | 1260 |
| AAVhu.39 (AAVLG-9) | 1261 |
| AAVhu.4 | 1262 |
| AAVhu.4 | 1263 |
| AAVhu.40 | 1264 |
| AAVhu.40 (AAV1 14.3) | 1265 |
| AAVhu.41 | 1266 |
| AAVhu.41 (AAV127.2) | 1267 |
| AAVhu.42 | 1268 |
| AAVhu.42 (AAV127.5) | 1269 |
| AAVhu.43 | 1270 |
| AAVhu.43 | 1271 |
| AAVhu.43 (AAV128.1) | 1272 |
| AAVhu.44 | 1273 |
| AAVhu.44 (AAV128.3) | 1274 |
| AAVhu.44R1 | 1275 |
| AAVhu.44R2 | 1276 |
| AAVhu.44R3 | 1277 |
| AAVhu.45 | 1278 |
| AAVhu.45 | 1279 |
| AAVhu.46 | 1280 |
| AAVhu.46 | 1281 |
| AAVhu.46 | 1282 |
| AAVhu.47 | 1283 |
| AAVhu.47 | 1284 |
| AAVhu.48 | 1285 |
| AAVhu.48 | 1286 |
| AAVhu.48 (AAV130.4) | 1287 |
| AAVhu.48R1 | 1288 |
| AAVhu.48R2 | 1289 |
| AAVhu.48R3 | 1290 |
| AAVhu.49 | 1291 |
| AAVhu.49 | 1292 |
| AAVhu.5 | 1293 |
| AAVhu.5 | 1294 |
| AAVhu.51 | 1295 |
| AAVhu.51 | 1296 |
| AAVhu.52 | 1297 |
| AAVhu.52 | 1298 |
| AAVhu.53 | 1299 |
| AAVhu.53 | 1300 |
| AAVhu.53 (AAV145.1) | 1301 |
| AAVhu.54 | 1302 |
| AAVhu.54 (AAV145.5) | 1303 |
| AAVhu.55 | 1304 |
| AAVhu.56 | 1305 |
| AAVhu.56 (AAV145.6) | 1306 |
| AAVhu.56 (AAV145.6) | 1307 |
| AAVhu.57 | 1308 |
| AAVhu.57 | 1309 |
| AAVhu.57 | 1310 |
| AAVhu.58 | 1311 |
| AAVhu.58 | 1312 |
| AAVhu.6 (AAV3.1) | 1313 |
| AAVhu.6 (AAV3.1) | 1314 |
| AAVhu.60 | 1315 |
| AAVhu.60 (AAV161.10) | 1316 |
| AAVhu.61 | 1317 |
| AAVhu.61 (AAV161.6) | 1318 |
| AAVhu.63 | 1319 |
| AAVhu.63 | 1320 |
| AAVhu.64 | 1321 |
| AAVhu.64 | 1322 |
| AAVhu.66 | 1323 |
| AAVhu.67 | 1324 |
| AAVhu.67 | 1325 |
| AAVhu.7 | 1326 |
| AAVhu.7 | 1327 |
| AAVhu.7 (AAV7.3) | 1328 |
| AAVhu.71 | 1329 |
| AAVhu.8 | 1330 |
| AAVhu.8 | 1331 |
| AAVhu.8 | 1332 |
| AAVhu.9 (AAV3.1) | 1333 |
| AAVhu.9 (AAV3.1) | 1334 |
| AAV-LKO1 | 1335 |
| AAV-LKO1 | 1336 |
| AAV-LK02 | 1337 |
| AAV-LK02 | 1338 |
| AAV-LK03 | 1339 |
| AAV-LK03 | 1340 |
| AAV-LK04 | 1341 |
| AAV-LK04 | 1342 |
| AAV-LK05 | 1343 |
| AAV-LK05 | 1344 |
| AAV-LK06 | 1345 |
| AAV-LK06 | 1346 |
| AAV-LK07 | 1347 |
| AAV-LK07 | 1348 |
| AAV-LK08 | 1349 |
| AAV-LK08 | 1350 |
| AAV-LK09 | 1351 |
| AAV-LK09 | 1352 |
| AAV-LK1O | 1353 |
| AAV-LK1O | 1354 |
| AAV-LK11 | 1355 |
| AAV-LK11 | 1356 |
| AAV-LK12 | 1357 |
| AAV-LK12 | 1358 |
| AAV-LK13 | 1359 |
| AAV-LK13 | 1360 |
| AAV-LK14 | 1361 |
| AAV-LK14 | 1362 |
| AAV-LK15 | 1363 |
| AAV-LK15 | 1364 |
| AAV-LK16 | 1365 |
| AAV-LK16 | 1366 |
| AAV-LK17 | 1367 |
| AAV-LK17 | 1368 |
| AAV-LK18 | 1369 |
| AAV-LK18 | 1370 |
| AAV-LK19 | 1371 |
| AAV-LK19 | 1372 |
| AAV-PAEC | 1373 |
| AAV-PAEC | 1374 |
| AAV-PAEC11 | 1375 |
| AAV-PAEC11 | 1376 |
| AAV-PAEC12 | 1377 |
| AAV-PAEC12 | 1378 |
| AAV-PAEC13 | 1379 |
| AAV-PAEC13 | 1380 |
| AAV-PAEC2 | 1381 |
| AAV-PAEC2 | 1382 |
| AAV-PAEC4 | 1383 |
| AAV-PAEC4 | 1384 |
| AAV-PAEC6 | 1385 |
| AAV-PAEC6 | 1386 |
| AAV-PAEC7 | 1387 |
| AAV-PAEC7 | 1388 |
| AAV-PAEC8 | 1389 |
| AAV-PAEC8 | 1390 |
| AAVpi.1 | 1391 |
| AAVpi.1 | 1392 |
| AAVpi.2 | 1393 |
| AAVpi.2 | 1394 |
| AAVpi.3 | 1395 |
| AAVpi.3 | 1396 |
| AAVrh.10 | 1397 |
| AAVrh.10 | 1398 |
| AAV44.2 | 1399 |
| AAVrh.10 (AAV44.2) | 1400 |
| AAV42.1B | 1401 |
| AAVrh.12 (AAV42.1b) | 1402 |
| AAVrh.13 | 1403 |
| AAVrh.13 | 1404 |
| AAVrh.13 | 1405 |
| AAVrh.13R | 1406 |
| AAV42.3A | 1407 |
| AAVrh.14 (AAV42.3a) | 1408 |
| AAV42.5A | 1409 |
| AAVrh.17 (AAV42.5a) | 1410 |
| AAV42.5B | 1411 |
| AAVrh.18 (AAV42.5b) | 1412 |
| AAV42.6B | 1413 |
| AAVrh.19 (AAV42.6b) | 1414 |
| AAVrh.2 | 1415 |
| AAVrh.2 | 1416 |
| AAVrh.20 | 1417 |
| AAV42.10 | 1418 |
| AAVrh.21 (AAV42.10) | 1419 |
| AAV42.11 | 1420 |
| AAVrh.22 (AAV42 .11) | 1421 |
| AAV42.12 | 1422 |
| AAVrh.23 (AAV42.12) | 1423 |
| AAV42.13 | 1424 |
| AAVrh.24 (AAV42.13) | 1425 |
| AAV42.15 | 1426 |
| AAVrh.25 (AAV42.15) | 1427 |
| AAVrh.2R | 1428 |
| AAVrh.31 (AAV223.1) | 1429 |
| AAVC1 | 1430 |
| AAVrh.32 (AAVC1) | 1431 |
| AAVrh.32/33 | 1432 |
| AAVrh.33 (AAVC3) | 1433 |
| AAVC5 | 1434 |
| AAVrh.34 (AAVC5) | 1435 |
| AAVF1 | 1436 |
| AAVrh.35 (AAVF1) | 1437 |
| AAVF3 | 1438 |
| AAVrh.36 (AAVF3) | 1439 |
| AAVrh.37 | 1440 |
| AAVrh.37 | 1441 |
| AAVrh.37 | 1442 |
| AAVrh.37R2 | 1443 |
| AAVrh.38 (AAVLG-4) | 1444 |
| AAVrh.38 (AAVLG-4) | 1445 |
| AAVrh.39 | 1446 |
| AAVrh.39 | 1447 |
| AAVrh.40 | 1448 |
| AAVrh.40 (AAVLG-10) | 1449 |
| AAVrh.43 (AAVN721-8) | 1450 |
| AAVrh.43 (AAVN721-8) | 1451 |
| AAVrh.44 | 1452 |
| AAVrh.44 | 1453 |
| AAVrh.45 | 1454 |
| AAVrh.45 | 1455 |
| AAVrh.46 | 1456 |
| AAVrh.46 | 1457 |
| AAVrh.47 | 1458 |
| AAVrh.47 (AAVbb.2) | 1459 |
| AAVrh.48 | 1460 |
| AAVrh.48.1 | 1461 |
| AAVrh.48.1.2 | 1462 |
| AAVrh.48.2 | 1463 |
| AAVrh.48 (AAV1-7) | 1464 |
| AAVrh.49 (AAV1-8) | 1465 |
| AAVrh.49 (AAV1-8) | 1466 |
| AAVrh.50 (AAV2-4) | 1467 |
| AAVrh.50 (AAV2-4) | 1468 |
| AAVrh.51 (AAV2-5) | 1469 |
| AAVrh.51 (AAV2-5) | 1470 |
| AAVrh.52 (AAV3-9) | 1471 |
| AAVrh.52 (AAV3-9) | 1472 |
| AAVrh.53 | 1473 |
| AAVrh.53 (AAV3-11) | 1474 |
| AAVrh.53 (AAV3-11) | 1475 |
| AAVrh.54 | 1476 |
| AAVrh.54 | 1477 |
| AAVrh.55 | 1478 |
| AAVrh.55 (AAV4-19) | 1479 |
| AAVrh.56 | 1480 |
| AAVrh.56 | 1481 |
| AAVrh.57 | 1482 |
| AAVrh.57 | 1483 |
| AAVrh.58 | 1484 |
| AAVrh.58 | 1485 |
| AAVrh.58 | 1486 |
| AAVrh.59 | 1487 |
| AAVrh.59 | 1488 |
| AAVrh.60 | 1489 |
| AAVrh.60 | 1490 |
| AAVrh.61 | 1491 |
| AAVrh.61 (AAV2-3) | 1492 |
| AAVrh.62 (AAV2-15) | 1493 |
| AAVrh.62 (AAV2-15) | 1494 |
| AAVrh.64 | 1495 |
| AAVrh.64 | 1496 |
| AAVrh.64 | 1497 |
| AAVRh.64R1 | 1498 |
| AAVRh.64R2 | 1499 |
| AAVrh.65 | 1500 |
| AAVrh.65 | 1501 |
| AAVrh.67 | 1502 |
| AAVrh.67 | 1503 |
| AAVrh.67 | 1504 |
| AAVrh.68 | 1505 |
| AAVrh.68 | 1506 |
| AAVrh.69 | 1507 |
| AAVrh.69 | 1508 |
| AAVrh.70 | 1509 |
| AAVrh.70 | 1510 |
| AAVrh.71 | 1511 |
| AAVrh.72 | 1512 |
| AAVrh.73 | 1513 |
| AAVrh.74 | 1514 |
| AAVrh.8 | 1515 |
| AAVrh.8 | 1516 |
| AAVrh.8R | 1517 |
| AAVrh.8R A586R mutant | 1518 |
| AAVrh.8R R533A mutant | 1519 |
| BAAV (bovine AAV) | 1520 |
| BAAV (bovine AAV) | 1521 |
| BAAV (bovine AAV) | 1522 |
| BAAV (bovine AAV) | 1523 |
| BAAV (bovine AAV) | 1524 |
| BAAV (bovine AAV) | 1525 |
| BAAV (bovine AAV) | 1526 |
| BAAV (bovine AAV) | 1527 |
| BAAV (bovine AAV) | 1528 |
| BAAV (bovine AAV) | 1529 |
| BAAV (bovine AAV) | 1530 |
| BAAV (bovine AAV) | 1531 |
| BAAV (bovine AAV) | 1532 |
| BNP61 AAV | 1533 |
| BNP61 AAV | 1534 |
| BNP62AAV | 1535 |
| BNP63 AAV | 1536 |
| caprine AAV | 1537 |
| caprine AAV | 1538 |
| true type AAV (ttAAV) | 1539 |
| AAAV (Avian AAV) | 1540 |
| AAAV (Avian AAV) | 1541 |
| AAAV (Avian AAV) | 1542 |
| AAAV (Avian AAV) | 1543 |
| AAAV (Avian AAV) | 1544 |
| AAAV (Avian AAV) | 1545 |
| AAAV (Avian AAV) | 1546 |
| AAAV (Avian AAV) | 1547 |
| AAAV (Avian AAV) | 1548 |
| AAAV (Avian AAV) | 1549 |
| AAAV (Avian AAV) | 1550 |
| AAAV (Avian AAV) | 1551 |
| AAAV (Avian AAV) | 1552 |
| AAAV (Avian AAV) | 1553 |
| AAAV (Avian AAV) | 1554 |
| AAV Shuffle 100-1 | 1555 |
| AAV Shuffle 100-1 | 1556 |
| AAV Shuffle 100-2 | 1557 |
| AAV Shuffle 100-2 | 1558 |
| AAV Shuffle 100-3 | 1559 |
| AAV Shuffle 100-3 | 1560 |
| AAV Shuffle 100-7 | 1561 |
| AAV Shuffle 100-7 | 1562 |
| AAV Shuffle 10-2 | 1563 |
| AAV Shuffle 10-2 | 1564 |
| AAV Shuffle 10-6 | 1565 |
| AAV Shuffle 10-6 | 1566 |
| AAV Shuffle 10-8 | 1567 |
| AAV Shuffle 10-8 | 1568 |
| AAVSM 100-10 | 1569 |
| AAVSM 100-10 | 1570 |
| AAVSM 100-3 | 1571 |
| AAVSM 100-3 | 1572 |
| AAVSM 10-1 | 1573 |
| AAVSM 10-1 | 1574 |
| AAVSM 10-2 | 1575 |
| AAVSM 10-2 | 1576 |
| AAVSM 10-8 | 1577 |
| AAVSM 10-8 | 1578 |
| AAVF1/HSC1 | 1579 |
| AAVF2/HSC2 | 1580 |
| AAVF3/HSC3 | 1581 |
| AAVF4/HSC4 | 1582 |
| AAVF5/HSC5 | 1583 |
| AAVF6/HSC6 | 1584 |
| AAVF7/HSC7 | 1585 |
| AAVF8/HSC8 | 1586 |
| AAVF9/HSC9 | 1587 |
| AAVF1 1/HSC11 | 1588 |
| AAVF12/HSC12 | 1589 |
| AAVF13/HSC13 | 1590 |
| AAVF14/HSC14 | 1591 |
| AAVF15/HSC15 | 1592 |
| AAVF16/HSC16 | 1593 |
| AAVF17/HSC17 | 1594 |
| AAVF1/HSC1 | 1595 |
| AAVF2/HSC2 | 1596 |
| AAVF3/HSC3 | 1597 |
| AAVF4/HSC4 | 1598 |
| AAVF5/HSC5 | 1599 |
| AAVF6/HSC6 | 1600 |
| AAVF7/HSC7 | 1601 |
| AAVF8/HSC8 | 1602 |
| AAVF9/HSC9 | 1603 |
| AAVF1 1/HSC1 1 | 1604 |
| AAVF12/HSC12 | 1605 |
| AAVF13/HSC13 | 1606 |
| AAVF14/HSC14 | 1607 |
| AAVF15/HSC15 | 1608 |
| AAVF16/HSC16 | 1609 |
| AAVF17/HSC17 | 1610 |
| AAVCBr-E1 | 1611 |
| AAVCBr-E2 | 1612 |
| AAVCBr-E3 | 1613 |
| AAVCBr-E4 | 1614 |
| AAVCBr-E5 | 1615 |
| AAVCBr-e5 | 1616 |
| AAVCBr-E6 | 1617 |
| AAVCBr-E7 | 1618 |
| AAVCBr-E8 | 1619 |
| AAVCLv-D1 | 1620 |
| AAVCLv-D2 | 1621 |
| AAVCLv-D3 | 1622 |
| AAVCLv-D4 | 1623 |
| AAVCLv-D5 | 1624 |
| AAVCLv-D6 | 1625 |
| AAVCLv-D7 | 1626 |
| AAVCLv-D8 | 1627 |
| AAVCLv-E1 | 1628 |
| AAVCLv-R1 | 1629 |
| AAVCLv-R2 | 1630 |
| AAVCLv-R3 | 1631 |
| AAVCLv-R4 | 1632 |
| AAVCLv-R5 | 1633 |
| AAVCLv-R6 | 1634 |
| AAVCLv-R7 | 1635 |
| AAVCLv-R8 | 1636 |
| AAVCLv-R9 | 1637 |
| AAVCLg-F1 | 1638 |
| AAVCLg-F2 | 1639 |
| AAVCLg-F3 | 1640 |
| AAVCLg-F4 | 1641 |
| AAVCLg-F5 | 1642 |
| AAVCLg-F6 | 1643 |
| AAVCLg-F7 | 1644 |
| AAVCLg-F8 | 1645 |
| AAVCSp-1 | 1646 |
| AAVCSp-10 | 1647 |
| AAVCSp-11 | 1648 |
| AAVCSp-2 | 1649 |
| AAVCSp-3 | 1650 |
| AAVCSp-4 | 1651 |
| AAVCSp-6 | 1652 |
| AAVCSp-7 | 1653 |
| AAVCSp-8 | 1654 |
| AAVCSp-9 | 1655 |
| AAVCHt-2 | 1656 |
| AAVCHt-3 | 1657 |
| AAVCKd-1 | 1658 |
| AAVCKd-10 | 1659 |
| AAVCKd-2 | 1660 |
| AAVCKd-3 | 1661 |
| AAVCKd-4 | 1662 |
| AAVCKd-6 | 1663 |
| AAVCKd-7 | 1664 |
| AAVCKd-8 | 1665 |
| AAVCLv-1 | 1666 |
| AAVCLv-12 | 1667 |
| AAVCLv-13 | 1668 |
| AAVCLv-2 | 1669 |
| AAVCLv-3 | 1670 |
| AAVCLv-4 | 1671 |
| AAVCLv-6 | 1672 |
| AAVCLv-8 | 1673 |
| AAVCKd-B1 | 1674 |
| AAVCKd-B2 | 1675 |
| AAVCKd-B3 | 1676 |
| AAVCKd-B4 | 1677 |
| AAVCKd-B5 | 1678 |
| AAVCKd-B6 | 1679 |
| AAVCKd-B7 | 1680 |
| AAVCKd-B8 | 1681 |
| AAVCKd-H1 | 1682 |
| AAVCKd-H2 | 1683 |
| AAVCKd-H3 | 1684 |
| AAVCKd-H4 | 1685 |
| AAVCKd-H5 | 1686 |
| AAVCKd-H6 | 1687 |
| AAV CHt-1 | 1688 |
| AAVCLv1-1 | 1689 |
| AAVCLv1-2 | 1690 |
| AAVCLv1-3 | 1691 |
| AAVCLv1-4 | 1692 |
| AAVC1v1-7 | 1693 |
| AAVC1v1-8 | 1694 |
| AAVC1v1-9 | 1695 |
| AAVC1v1-10 | 1696 |
| AAV.VR-355 | 1697 |
| AAV.hu.48R3 | 1698 |
| AAVCBr-E1 | 1699 |
| AAVCBr-E2 | 1700 |
| AAVCBr-E3 | 1701 |
| AAVCBr-E4 | 1702 |
| AAVCBr-E5 | 1703 |
| AAVCBr-e5 | 1704 |
| AAVCBr-E6 | 1705 |
| AAVCBr-E7 | 1706 |
| AAVCBr-E8 | 1707 |
| AAVCLv-D1 | 1708 |
| AAVCLv-D2 | 1709 |
| AAVCLv-D3 | 1710 |
| AAVCLv-D4 | 1711 |
| AAVCLv-D5 | 1712 |
| AAVCLv-D6 | 1713 |
| AAVCLv-D7 | 1714 |
| AAVCLv-D8 | 1715 |
| AAVCLv-E1 | 1716 |
| AAVCLv-R1 | 1717 |
| AAVCLv-R2 | 1718 |
| AAVCLv-R3 | 1719 |
| AAVCLv-R4 | 1720 |
| AAVCLv-R5 | 1721 |
| AAVCLv-R6 | 1722 |
| AAVCLv-R7 | 1723 |
| AAVCLv-R8 | 1724 |
| AAVCLv-R9 | 1725 |
| AAVCLg-F1 | 1726 |
| AAVCLg-F2 | 1727 |
| AAVCLg-F3 | 1728 |
| AAVCLg-F4 | 1729 |
| AAVCLg-F5 | 1730 |
| AAVCLg-F6 | 1731 |
| AAVCLg-F7 | 1732 |
| AAVCLg-F8 | 1733 |
| AAVCSp-1 | 1734 |
| AAVCSp-10 | 1735 |
| AAVCSp-11 | 1736 |
| AAVCSp-2 | 1737 |
| AAVCSp-3 | 1738 |
| AAVCSp-4 | 1739 |
| AAVCSp-6 | 1740 |
| AAVCSp-7 | 1741 |
| AAVCSp-8 | 1742 |
| AAVCSp-9 | 1743 |
| AAVCHt-2 | 1744 |
| AAVCHt-3 | 1745 |
| AAVCKd-1 | 1746 |
| AAVCKd-10 | 1747 |
| AAVCKd-2 | 1748 |
| AAVCKd-3 | 1749 |
| AAVCKd-4 | 1750 |
| AAVCKd-6 | 1751 |
| AAVCKd-7 | 1752 |
| AAVCKd-8 | 1753 |
| AAVCLv-1 | 1754 |
| AAVCLv-12 | 1755 |
| AAVCLv-13 | 1756 |
| AAVCLv-2 | 1757 |
| AAVCLv-3 | 1758 |
| AAVCLv-4 | 1759 |
| AAVCLv-6 | 1760 |
| AAVCLv-8 | 1761 |
| AAVCKd-B1 | 1762 |
| AAVCKd-B2 | 1763 |
| AAVCKd-B3 | 1764 |
| AAVCKd-B4 | 1765 |
| AAVCKd-B5 | 1766 |
| AAVCKd-B6 | 1767 |
| AAVCKd-B7 | 1768 |
| AAVCKd-B8 | 1769 |
| AAVCKd-H1 | 1770 |
| AAVCKd-H2 | 1771 |
| AAVCKd-H3 | 1772 |
| AAVCKd-H4 | 1773 |
| AAVCKd-H5 | 1774 |
| AAVCKd-H6 | 1775 |
| AAVCHt-1 | 1776 |
| AAVCHt-P2 | 1777 |
| AAVCHt-P5 | 1778 |
| AAVCHt-P9 | 1779 |
| AAVCBr-7.1 | 1780 |
| AAVCBr-7.2 | 1781 |
| AAVCBr-7.3 | 1782 |
| AAVCBr-7.4 | 1783 |
| AAVCBr-7.5 | 1784 |
| AAVCBr-7.7 | 1785 |
| AAVCBr-7.8 | 1786 |
| AAV CBr-7.10 | 1787 |
| AAVCKd-N3 | 1788 |
| AAVCKd-N4 | 1789 |
| AAVCKd-N9 | 1790 |
| AAVCLv-L4 | 1791 |
| AAVCLv-L5 | 1792 |
| AAVCLv-L6 | 1793 |
| AAVCLv-K1 | 1794 |
| AAVCLv-K3 | 1795 |
| AAVCLv-K6 | 1796 |
| AAVCLv-M1 | 1797 |
| AAVCLv-M11 | 1798 |
| AAVCLv-M2 | 1799 |
| AAVCLv-M5 | 1800 |
| AAVCLv-M6 | 1801 |
| AAVCLv-M7 | 1802 |
| AAVCLv-M8 | 1803 |
| AAVCLv-M9 | 1804 |
| AAVCHt-P1 | 1805 |
| AAVCHt-P6 | 1806 |
| AAVCHt-P8 | 1807 |
| AAVCHt-6.1 | 1808 |
| AAV CHt-6.10 | 1809 |
| AAVCHt-6.5 | 1810 |
| AAVCHt-6.6 | 1811 |
| AAVCHt-6.7 | 1812 |
| AAVCHt-6.8 | 1813 |
| AAVCSp-8.10 | 1814 |
| AAVCSp-8.2 | 1815 |
| AAVCSp-8.4 | 1816 |
| AAVCSp-8.5 | 1817 |
| AAVCSp-8.6 | 1818 |
| AAVCSp-8.7 | 1819 |
| AAVCSp-8.8 | 1820 |
| AAVCSp-8.9 | 1821 |
| AAVCBr-B7.3 | 1822 |
| AAVCBr-B7.4 | 1823 |
| AAV3B | 1824 |
| AAV4 | 1825 |
| AAV5 | 1826 |
| AAVCHt-P2 | 1827 |
| AAVCHt-P5 | 1828 |
| AAVCHt-P9 | 1829 |
| AAVCBr-7.1 | 1830 |
| AAVCBr-7.2 | 1831 |
| AAVCBr-7.3 | 1832 |
| AAVCBr-7.4 | 1833 |
| AAVCBr-7.5 | 1834 |
| AAVCBr-7.7 | 1835 |
| AAVCBr-7.8 | 1836 |
| AAV CBr-7.10 | 1837 |
| AAVCKd-N3 | 1838 |
| AAVCKd-N4 | 1839 |
| AAVCKd-N9 | 1840 |
| AAV CLv-L4 | 1841 |
| AAVCLv-L5 | 1842 |
| AAVCLv-L6 | 1843 |
| AAVCLv-K1 | 1844 |
| AAVCLv-K3 | 1845 |
| AAV CLv-K6 | 1846 |
| AAVCLv-M1 | 1847 |
| AAVCLv-M11 | 1848 |
| AAVCLv-M2 | 1849 |
| AAVCLv-M5 | 1850 |
| AAVCLv-M6 | 1851 |
| AAVCLv-M7 | 1852 |
| AAVCLv-M8 | 1853 |
| AAVCLv-M9 | 1854 |
| AAVCHt-P1 | 1855 |
| AAVCHt-P6 | 1856 |
| AAVCHt-P8 | 1857 |
| AAVCHt-6.1 | 1858 |
| AAV CHt-6.10 | 1859 |
| AAVCHt-6.5 | 1860 |
| AAVCHt-6.6 | 1861 |
| AAVCHt-6.7 | 1862 |
| AAVCHt-6.8 | 1863 |
| AAVCSp-8.10 | 1864 |
| AAVCSp-8.2 | 1865 |
| AAVCSp-8.4 | 1866 |
| AAVCSp-8.5 | 1867 |
| AAVCSp-8.6 | 1868 |
| AAV CSp-8.7 | 1869 |
| AAVCSp-8.8 | 1870 |
| AAVCSp-8.9 | 1871 |
| AAV CBr-B7.3 | 1872 |
| AAV CBr-B7.4 | 1873 |
| AAV3B | 1874 |
| AAV4 | 1875 |
| AAV5 | 1876 |
| GPV | 1877 |
| B19 | 1878 |
| MVM | 1879 |
| FPV | 1880 |
| CPV | 1881 |
| AAV6 | 1882 |
| AAV6 | 1883 |
| AAV2 | 1884 |
| ShH1O | 1885 |
| ShH13 | 1886 |
| ShH1O | 1887 |
| ShH1O | 1888 |
| ShH1O | 1889 |
| ShH1O | 1890 |
| ShH1O | 1891 |
| rh74 | 1892 |
| rh74 | 1893 |
| AAV8 | 1894 |
| rh74 | 1895 |
| rh74 (RHM4-1) | 1896 |
| rh74 (RHM15-1) | 1897 |
| rh74 (RHM15-2) | 1898 |
| rh74 (RHM15-3/RHM15-5) | 1899 |
| rh74 (RHM15-4) | 1900 |
| rh74 (RHM15-6) | 1901 |
| rh74 (RHM4-1) | 1902 |
| rh74 (RHM15-1) | 1903 |
| rh74 (RHM15-2) | 1904 |
| rh74 (RHM15-3/RHM15-5) | 1905 |
| rh74 (RHM15-4) | 1906 |
| rh74 (RHM15-6) | 1907 |
| AAV2 (comprising lung | 1908 |
| specific polypeptide) | |
| AAV2 (comprising lung | 1909 |
| specific polypeptide) | |
| Anc80 | 1910 |
| Anc80 | 1911 |
| Anc81 | 1912 |
| Anc80 | 1913 |
| Anc82 | 1914 |
| Anc82 | 1915 |
| Anc83 | 1916 |
| Anc83 | 1917 |
| Anc84 | 1918 |
| Anc84 | 1919 |
| Anc94 | 1920 |
| Anc94 | 1921 |
| Anc113 | 1922 |
| Anc113 | 1923 |
| Anc126 | 1924 |
| Anc126 | 1925 |
| Anc127 | 1926 |
| Anc127 | 1927 |
| Anc80L27 | 1928 |
| Anc80L59 | 1929 |
| Anc80L60 | 1930 |
| Anc80L62 | 1931 |
| Anc80L65 | 1932 |
| Anc80L33 | 1933 |
| Anc80L36 | 1934 |
| Anc80L44 | 1935 |
| Anc80L1 | 1936 |
| Anc80L1 | 1937 |
| AAV-X1 | 1938 |
| AAV-X1b | 1939 |
| AAV-X5 | 1940 |
| AAV-X19 | 1941 |
| AAV-X21 | 1942 |
| AAV-X22 | 1943 |
| AAV-X23 | 1944 |
| AAV-X24 | 1945 |
| AAV-X25 | 1946 |
| AAV-X26 | 1947 |
| AAV-X1 | 1948 |
| AAV-X1b | 1949 |
| AAV-X5 | 1950 |
| AAV-X19 | 1951 |
| AAV-X21 | 1952 |
| AAV-X22 | 1953 |
| AAV-X23 | 1954 |
| AAV-X24 | 1955 |
| AAV-X25 | 1956 |
| AAV-X26 | 1957 |
| AAVrh8 | 1958 |
| AAVrh8VP2FC5 | 1959 |
| AAVrh8VP2FC44 | 1960 |
| AAVrh8VP2ApoB100 | 1961 |
| AAVrh8VP2RVG | 1962 |
| AAVrh8VP2Angiopep-2 VP2 | 1963 |
| AAV9.47VP1.3 | 1964 |
| AAV9.47VP2ICAMg3 | 1965 |
| AAV9.47VP2RVG | 1966 |
| AAV9.47VP2Angiopep-2 | 1967 |
| AAV9.47VP2A-string | 1968 |
| AAVrh8VP2FC5 VP2 | 1969 |
| AAVrh8VP2FC44 VP2 | 1970 |
| AAVrh8VP2ApoB100 VP2 | 1971 |
| AAVrh8VP2RVG VP2 | 1972 |
| AAVrh8VP2Angiopep-2 VP2 | 1973 |
| AAV9.47VP2ICAMg3 VP2 | 1974 |
| AAV9.47VP2RVG VP2 | 1975 |
| AAV9.47VP2Angiopep-2 VP2 | 1976 |
| AAV9.47VP2A-string VP2 | 1977 |
| rAAV-B1 | 1978 |
| rAAV-B2 | 1979 |
| rAAV-B3 | 1980 |
| rAAV-B4 | 1981 |
| rAAV-B1 | 1982 |
| rAAV-B2 | 1983 |
| rAAV-B3 | 1984 |
| rAAV-B4 | 1985 |
| rAAV-L1 | 1986 |
| rAAV-L2 | 1987 |
| rAAV-L3 | 1988 |
| rAAV-L4 | 1989 |
| rAAV-L1 | 1990 |
| rAAV-L2 | 1991 |
| rAAV-L3 | 1992 |
| rAAV-L4 | 1993 |
| AAV9 | 1994 |
| rAAV | 1995 |
| rAAV | 1996 |
| rAAV | 1997 |
| rAAV | 1998 |
| rAAV | 1999 |
| rAAV | 2000 |
| rAAV | 2001 |
| rAAV | 2002 |
| rAAV | 2003 |
| rAAV | 2004 |
| rAAV | 2005 |
| rAAV | 2006 |
| rAAV | 2007 |
| rAAV | 2008 |
| rAAV | 2009 |
| rAAV | 2010 |
| rAAV | 2011 |
| rAAV | 2012 |
| rAAV | 2013 |
| rAAV | 2014 |
| rAAV | 2015 |
| rAAV | 2016 |
| rAAV | 2017 |
| rAAV | 2018 |
| rAAV | 2019 |
| rAAV | 2020 |
| rAAV | 2021 |
| rAAV | 2022 |
| rAAV | 2023 |
| rAAV | 2024 |
| rAAV | 2025 |
| rAAV | 2026 |
| rAAV | 2027 |
| rAAV | 2028 |
| rAAV | 2029 |
| rAAV | 2030 |
| rAAV | 2031 |
| rAAV | 2032 |
| rAAV | 2033 |
| rAAV | 2034 |
| rAAV | 2035 |
| rAAV | 2036 |
| rAAV | 2037 |
| rAAV | 2038 |
| rAAV | 2039 |
| rAAV | 2040 |
| rAAV | 2041 |
| rAAV | 2042 |
| rAAV | 2043 |
| rAAV | 2044 |
| rAAV | 2045 |
| rAAV | 2046 |
| rAAV | 2047 |
| rAAV | 2048 |
| rAAV | 2049 |
| rAAV | 2050 |
| rAAV | 2051 |
| rAAV | 2052 |
| rAAV | 2053 |
| rAAV | 2054 |
| rAAV | 2055 |
| rAAV | 2056 |
| rAAV | 2057 |
| rAAV | 2058 |
| rAAV | 2059 |
| rAAV | 2060 |
| rAAV | 2061 |
| rAAV | 2062 |
| rAAV | 2063 |
| rAAV | 2064 |
| rAAV | 2065 |
| rAAV | 2066 |
| rAAV | 2067 |
| rAAV | 2068 |
| rAAV | 2069 |
| rAAV | 2070 |
| rAAV | 2071 |
| rAAV | 2072 |
| rAAV | 2073 |
| rAAV | 2074 |
| rAAV | 2075 |
| rAAV | 2076 |
| rAAV | 2077 |
| rAAV | 2078 |
| rAAV | 2079 |
| rAAV | 2080 |
| rAAV | 2081 |
| rAAV | 2082 |
| rAAV | 2083 |
| rAAV | 2084 |
| rAAV | 2085 |
| rAAV | 2086 |
| rAAV | 2087 |
| rAAV | 2088 |
| rAAV | 2089 |
| rAAV | 2090 |
| rAAV | 2091 |
| rAAV | 2092 |
| rAAV | 2093 |
| rAAV | 2094 |
| rAAV | 2095 |
| rAAV | 2096 |
| rAAV | 2097 |
| rAAV | 2098 |
| rAAV | 2099 |
| rAAV | 2100 |
| rAAV | 2101 |
| rAAV | 2102 |
| rAAV | 2103 |
| rAAV | 2104 |
| rAAV | 2105 |
| rAAV | 2106 |
| rAAV | 2107 |
| rAAV | 2108 |
| rAAV | 2109 |
| rAAV | 2110 |
| rAAV | 2111 |
| rAAV | 2112 |
| rAAV | 2113 |
| rAAV | 2114 |
| rAAV | 2115 |
| rAAV | 2116 |
| rAAV | 2117 |
| rAAV | 2118 |
| rAAV | 2119 |
| rAAV | 2120 |
| rAAV | 2121 |
| rAAV | 2122 |
| AAV8E532K | 2123 |
| AAV8E532K | 2124 |
| rAAV4 | 2125 |
| rAAV4 | 2126 |
| rAAV4 | 2127 |
| rAAV4 | 2128 |
| rAAV4 | 2129 |
| rAAV4 | 2130 |
| rAAV4 | 2131 |
| rAAV4 | 2132 |
| rAAV4 | 2133 |
| rAAV4 | 2134 |
| rAAV4 | 2135 |
| rAAV4 | 2136 |
| rAAV4 | 2137 |
| rAAV4 | 2138 |
| rAAV4 | 2139 |
| rAAV4 | 2140 |
| rAAV4 | 2141 |
| rAAV4 | 2142 |
| rAAV4 | 2143 |
| rAAV4 | 2144 |
| AAV11 | 2145 |
| AAV12 | 2146 |
| rh32 | 2147 |
| Th33 | 2148 |
| Th34 | 2149 |
| rAAV4 | 2150 |
| rAAV4 | 2151 |
| rAAV4 | 2152 |
| rAAV4 | 2153 |
| rAAV4 | 2154 |
| rAAV4 | 2155 |
| AAV2/8 | 2156 |
| AAV2/8 | 2157 |
| ancestral AAV | 2158 |
| ancestral AAV variant C4 | 2159 |
| ancestral AAV variant C7 | 2160 |
| ancestral AAV variant G4 | 2161 |
| consensus amino acid | 2162 |
| sequence of ancestral AAV | |
| variants, C4, C7 and G4 | |
| consensus amino acid | 2163 |
| sequence of ancestral AAV | |
| variants, C4 and C7 | |
| AAVS (with an AAV2 | 2164 |
| phospholipase domain) | |
| AAVVR-942n | 2165 |
| AAVS-A (M569V) | 2166 |
| AAVS-A (M569V) | 2167 |
| AAVS-A (Y585V) | 2168 |
| AAVS-A (Y585V) | 2169 |
| AAVS-A (L587T) | 2170 |
| AAVS-A (L587T) | 2171 |
| AAVS-A (Y585V/L587T) | 2172 |
| AAVS-A (Y585V/L587T) | 2173 |
| AAV5-B (D652A) | 2174 |
| AAV5-B (D652A) | 2175 |
| AAV5-B (T362M) | 2176 |
| AAV5-B (T362M) | 2177 |
| AAV5-B (Q359D) | 2178 |
| AAV5-B (Q359D) | 2179 |
| AAV5-B (E350Q) | 2180 |
| AAV5-B (E350Q) | 2181 |
| AAV5-B (P533S) | 2182 |
| AAV5-B (P533S) | 2183 |
| AAV5-B (P533G) | 2184 |
| AAV5-B (P533G) | 2185 |
| AAVS-mutation in loop V11 | 2186 |
| AAVS-mutation in loop V11 | 2187 |
| AAVS | 2188 |
| Mut A (LK03/AAVS) | 2189 |
| Mut B (LK03/AAVS) | 2190 |
| Mut C (AAV8/AAV3B) | 2191 |
| MutD (AAV5/AAV3B) | 2192 |
| Mut E (AAV8/AAV3B) | 2193 |
| Mut F (AAV3B/AAV8) | 2194 |
| AAV44.9 | 2195 |
| AAV44.9 | 2196 |
| AAVrh8 | 2197 |
| AAV44.9 (S470N) | 2198 |
| Th74 VP1 | 2199 |
| AAV-LK03 (L125I) | 2200 |
| AAV3B (S663V + T492V) | 2201 |
| Anc80 | 2202 |
| Anc80 | 2203 |
| Anc81 | 2204 |
| Anc81 | 2205 |
| Anc82 | 2206 |
| Anc82 | 2207 |
| Anc83 | 2208 |
| Anc83 | 2209 |
| Anc84 | 2210 |
| Anc84 | 2211 |
| Anc94 | 2212 |
| Anc94 | 2213 |
| Anc113 | 2214 |
| Anc113 | 2215 |
| Anc126 | 2216 |
| Anc126 | 2217 |
| Anc127 | 2218 |
| Anc127 | 2219 |
| Anc80L27 | 2220 |
| Anc80L59 | 2221 |
| Anc80L60 | 2222 |
| Anc80L62 | 2223 |
| Anc80L65 | 2224 |
| Anc80L33 | 2225 |
| Anc80L36 | 2226 |
| Anc80L44 | 2227 |
| Anc80L1 | 2228 |
| Anc80L1 | 2229 |
| AAVrh1O | 2230 |
| Anc11O | 2231 |
| Anc11O | 2232 |
| AAVrh32.33 | 2233 |
| AAVrh74 | 2234 |
| AAV2 | 2235 |
| AAV2 | 2236 |
| AAV2 | 2237 |
| Pallo-like vims | 2238 |
| Pallo-like vims | 2239 |
| Pallo-like vims | 2240 |
| Pallo-like vims | 2241 |
| Pallo-like vims | 2242 |
| Pallo-like vims | 2243 |
| AAVrh.10 | 2244 |
| AAVrh.10 | 2245 |
| AAV2tYF | 2246 |
| AAV-SPK | 2247 |
| AAV2.5 | 2248 |
| AAV1.1 | 2249 |
| AAV6.1 | 2250 |
| AAV6.3.1 | 2251 |
| AAV2i8 | 2252 |
| AAV2i8 | 2253 |
| ttAAV | 2254 |
| ttAAV-S312N | 2255 |
| ttAAV-S312N | 2256 |
| AAV6 (Y705, Y731, and T492) | 2257 |
| AAV2 | 2258 |
| AAV2 | 2259 |

The single-stranded DNA genome of wild-type AAV is about 4.7 kilobases (kb). In practice, AAV genomes of up to about 5.0 kb appear to be completely packaged, i.e., be full-length, into AAV virus particles. With two AAV inverted terminal repeats (ITRs) of about 145 bases, the DNA packaging capacity of an AAV vector is such that a maximum of about 4.4 kb of protein.

The wild-type AAV genome comprises two open reading frames, Rep and Cap, flanked by two inverted terminal repeats (ITRs). Typically, when producing a recombinant AAV, the sequence between the two ITRs is replaced with one or more sequence of interest (e.g., a transgene), and the Rep and Cap sequences are provided in trans. The recombinant AAV genome construct, comprising two ITRs flanking a sequence of interest (such as a transgene), is referred to herein as an AAV expression cassette. The disclosure provides AAV expression cassettes for production of AAV viral vectors.

In some embodiments, an AAV expression cassette comprises a nucleic acid of the disclosure, i.e., a nucleic acid comprising a sequence encoding a single guide RNA (sgRNA) comprising a spacer sequence and a scaffold sequence wherein the spacer sequence targets an exon sequence of the DMD gene, such as a sequence of exon 43, 44, 45, 50, or 53 of the DMD gene.

In some embodiments, an AAV expression cassette comprises a first ITR, a transgene sequence, and a second ITR. In some embodiments, an AAV expression cassette comprises a first ITR, an expression control sequence (such as a promoter or enhancer), a transgene sequence, and a second ITR. In some embodiments, an AAV expression cassette comprises a first ITR, an expression control sequence (such as a promoter or enhancer), a transgene sequence, a stuffer sequence, and a second ITR. The transgene may comprise all or part of a nucleic acid of the disclosure. For example, the transgene may comprise a gRNA sequence (i.e., spacer+scaffold sequences), wherein the gRNA targets an exon sequence of the DMD gene, such as a sequence of exon 43, 44, 45, 50, or 53 of the DMD gene.

In some embodiments, an AAV expression cassette comprises a first ITR, a gRNA sequence, and a second ITR. In some embodiments, an AAV expression cassette comprises a first ITR, an expression control sequence (such as a promoter or enhancer), a gRNA sequence, and a second ITR. In some embodiments, an AAV expression cassette comprises a first ITR, an expression control sequence (such as a promoter or enhancer), a gRNA sequence, a stuffer sequence, and a second ITR.

In some embodiments, the transgene comprises more than one guide RNA sequence, such as two, three, four, five, six, seven, or eight gRNA sequences. In some embodiments, the transgene comprises three, four or five gRNA sequences. In some embodiments, each gRNA sequence is operably linked to an expression control sequence (such as a promoter or enhancer).

In some embodiments, an AAV expression cassette comprises a first ITR, a first expression control sequence (such as a promoter or enhancer), a first gRNA sequence, a second expression control sequence (such as a promoter or enhancer), a second gRNA sequence, and a second ITR.

In some embodiments, an AAV expression cassette comprises a first ITR, a first expression control sequence (such as a promoter or enhancer), a first gRNA sequence, a second expression control sequence (such as a promoter or enhancer), a second gRNA sequence, a third expression control sequence (such as a promoter or enhancer), a third gRNA sequence, and a second ITR.

In some embodiments, an AAV expression cassette comprises a first ITR, a first expression control sequence (such as a promoter or enhancer), a first gRNA sequence, a second expression control sequence (such as a promoter or enhancer), a second gRNA sequence, a third expression control sequence (such as a promoter or enhancer), a third gRNA sequence, a fourth expression control sequence (such as a promoter or enhancer), a fourth gRNA sequence, and a second ITR.

In some embodiments, an AAV expression cassette comprises a first ITR, a first expression control sequence (such as a promoter or enhancer), a first gRNA sequence, a second expression control sequence (such as a promoter or enhancer), a second gRNA sequence, a third expression control sequence (such as a promoter or enhancer), a third gRNA sequence, a fourth expression control sequence (such as a promoter or enhancer), a fourth gRNA sequence, a fifth expression control sequence (such as a promoter or enhancer), a fifth gRNA sequence, and a second ITR.

In some embodiments, all of the gRNA sequences are the same. In some embodiments, two or more of the gRNA sequences are different. In some embodiments, all of the gRNA sequences are different. In some embodiments, the AAV expression cassette further comprises a stuffer sequence. In some embodiments, the AAV expression cassette further comprises a polyadenosine (polyA) sequence.

In some embodiments, an AAV expression cassette comprises sequences encoding a first ITR, a first promoter, a first gRNA comprising a first spacer sequence, a second promoter, a second gRNA comprising a second spacer sequence, a third promoter, a third gRNA comprising a third spacer sequence; and a second ITR. At least one of the first, second, and third spacer sequences may target a sequence of the DMD gene (e.g., exon 43, exon 44, exon 46, exon 50 or exon 53 of the DMD gene). In some embodiments, the first, second, and third spacer sequences are each individually selected from any one of the gRNA spacer sequences in Table 2, or a sequence at least 95% identical thereto. In some embodiments, at least two of the first, second, and third spacer sequences are different. In some embodiments, the first, second, and third spacer sequences are the same. In some embodiments, the first, second, and/or third spacer sequences have a sequence that is at least 95% identical or 100% identical to the sequence of any one of SEQ ID NOs: 135-146, 170-260, 337-339, or 617. In some embodiments, the first, second, and/or third spacer sequences have a sequence that is at least 95% identical or 100% identical to the sequence of any one of SEQ ID NOs: 135-146, 170-260, 337-339, or 617.

In some embodiments, an AAV expression cassette comprises a first gRNA comprising a first spacer sequence, a second gRNA comprising a second spacer sequence, a third gRNA comprising a third spacer sequence, and a fourth gRNA comprising a fourth spacer sequence. In some embodiments, two, three, or four of the gRNAs are the same. In some embodiments, two, three, or four of the gRNAs are different. In some embodiments, an AAV expression cassette comprises a first promoter, a first gRNA comprising a first spacer sequence, a second promoter, a second gRNA comprising a second spacer sequence, a third promoter, a third gRNA comprising a third spacer sequence, a fourth promoter, and a fourth gRNA comprising a fourth spacer sequence. In some embodiments, an AAV expression cassette comprises a first ITR, a first promoter, a first gRNA comprising a first spacer sequence, a second promoter, a second gRNA comprising a second spacer sequence, a third promoter, a third gRNA comprising a third spacer sequence, a fourth promoter, a fourth gRNA comprising a fourth spacer sequence, and a second ITR. In some embodiments, the expression cassette further comprises a stuffer sequence.

In some embodiments, an AAV expression cassette comprises a first gRNA comprising a first spacer sequence, a second gRNA comprising a second spacer sequence, a third gRNA comprising a third spacer sequence, a fourth gRNA comprising a fourth spacer sequence, and a fifth gRNA comprising a fifth spacer sequence. In some embodiments, two, three, four, or five of the gRNAs are the same. In some embodiments, two, three, four or five of the gRNAs are different. In some embodiments, an AAV expression cassette comprises a first promoter, a first gRNA comprising a first spacer sequence, a second promoter, a second gRNA comprising a second spacer sequence, a third promoter, a third gRNA comprising a third spacer sequence, a fourth promoter, a fourth gRNA comprising a fourth spacer sequence, a fifth promoter, and a fifth gRNA comprising a fifth spacer sequence. In some embodiments, an AAV expression cassette comprises a first ITR, a first promoter, a first gRNA comprising a first spacer sequence, a second promoter, a second gRNA comprising a second spacer sequence, a third promoter, a third gRNA comprising a third spacer sequence, a fourth promoter, a fourth gRNA comprising a fourth spacer sequence, a fifth promoter, a fifth gRNA comprising a fifth spacer sequence, and a second ITR. In some embodiments, the expression cassette further comprises a stuffer sequence.

In some embodiments, an AAV expression cassette comprises sequences encoding a first inverted terminal repeat (ITR), a first promoter, a first gRNA comprising a first spacer sequence (e.g., a sequence at least 95% or 100% identical to any one of SEQ ID NOs: 135-146, 170-260, 337-339, or 617) and a scaffold sequence (e.g., a scaffold sequence at least 95% or 100% identical to any of SEQ ID NO: 147 to 153); and a second ITR.

In some embodiments, an AAV expression cassette comprises sequences encoding a first inverted terminal repeat (ITR), a first promoter, a first gRNA comprising a first spacer sequence (e.g., a sequence at least 95% or 100% identical to any one of SEQ ID NOs: 135-146, 170-260, 337-339, or 617) and a scaffold sequence (e.g., a scaffold sequence at least 95% or 100% identical to any of SEQ ID NO: 147 to 153), a second promoter, a second gRNA comprising a second spacer sequence (e.g., a sequence at least 95% or 100% identical to any one of SEQ ID NOs: 135-146, 170-260, 337-339, or 617) and a scaffold sequence (e.g., a scaffold sequence at least 95% or 100% identical to any of SEQ ID NO: 147 to 153); and a second ITR.

In some embodiments, an AAV expression cassette comprises sequences encoding a first inverted terminal repeat (ITR), a first promoter, a first gRNA comprising a first spacer sequence (e.g., a sequence at least 95% or 100% identical to any one of SEQ ID NOs: 135-146, 170-260, 337-339, or 617) and a scaffold sequence (e.g., a scaffold sequence at least 95% or 100% identical to any of SEQ ID NO: 147 to 153), a second promoter, a second gRNA comprising a second spacer sequence (e.g., a sequence at least 95% or 100% identical to any one of SEQ ID NOs: 135-146, 170-260, 337-339, or 617) and a scaffold sequence (e.g., a scaffold sequence at least 95% or 100% identical to any of SEQ ID NO: 147 to 153), a third promoter, a third gRNA comprising a third spacer sequence (e.g., a sequence at least 95% or 100% identical to any one of SEQ ID NOs: 135-146, 170-260, 337-339, or 617) and a scaffold sequence (e.g., a scaffold sequence at least 95% or 100% identical to any of SEQ ID NO: 147 to 153); and a second ITR.

In some embodiments, an AAV expression cassette comprises a first inverted terminal repeat (ITR), a first promoter, a nucleic acid comprising a gRNA targeting a sequence of the DMD gene, such as a sequence of Exon 43, 44, 45, 50, or 53 of the DMD gene, and a second ITR. In some embodiments, the AAV expression cassette further comprises a polyadenosine (polyA) sequence.

In some embodiments, one or both of the first ITR and the second ITR are isolated or derived from any one of AAV1, AAV2, AAV3, AAV4, AAV5, AAV6, AAV7, AAV8, AAV9, AAV10, AAV11, AAV12, AAVRh74, AAV2i8, AAVRh10, AAV39, AAV43, AAVRh8, avian AAV, bovine AAV, canine AAV, equine AAV, and ovine AAV. In some embodiments, the expression cassette comprises multiple copies of the gRNA, such as 2, 3, 4, or 5 copies of the gRNA.

In some embodiments, an AAV expression cassette comprises a sequence to make the AAV vector less immunogenic (e.g., a “cloaking” sequence). In some embodiments, the sequence is isolated or derived from a telomere sequence. In some embodiments, the nucleotide sequence binds to a toll-like receptor, such as TLR9.

In some embodiments, an AAV expression cassette comprises sequences encoding a first ITR, a first promoter, a first gRNA comprising a first spacer sequence, a second promoter, a second gRNA comprising the first spacer sequence, a third promoter, a third gRNA comprising the first spacer sequence, and a second ITR.

In some embodiments, an AAV expression cassette comprises sequences encoding a first ITR, a first promoter, a first gRNA comprising a first spacer sequence, a second promoter, a second gRNA comprising the first spacer sequence, a third promoter, a third gRNA comprising the first spacer sequence, (optionally) a first stuffer sequence, and a second ITR. The first spacer sequence may target the DMD gene, for example it may target exon 43, exon 44, exon 46, exon 50 or exon 53 of the DMD gene. In some embodiments, the first spacer sequence is selected from any one of the gRNA sequences in Table 2, or a sequence at least 95% identical thereto.

The AAV expression cassettes described herein may be incorporated into an AAV vector. In some embodiments, an AAV vector comprises an AAV expression cassette encapsidated by an AAV capsid protein.

In some embodiments, the AAV vector is based on one or more of AAV1, AAV2, AAV3, AAV4, AAV5, AAV6, AAV7, AAV8, AAV9, AAV10, AAV11, AAV12, AAVRh74, AAV2i8, AAVRh10, AAV39, AAV43, AAVRh8, avian AAV, bovine AAV, canine AAV, equine AAV, or ovine AAV. In some embodiments, the AAV vector is based on a modified AAV, comprising one or more non-naturally occurring sequences. In some embodiments, the AAV vector is based on a chimeric AAV. The AAV vector may be replication-defective or conditionally replication defective.

Adenoviruses. “Adenovirus expression vector” is meant to include those constructs containing adenovirus sequences sufficient to (a) support packaging of the construct and (b) to express an antisense polynucleotide that has been cloned therein. In this context, expression does not require that the gene product be synthesized.

The expression vector comprises a genetically engineered form of adenovirus. Knowledge of the genetic organization of adenovirus, a 36 kB, linear, double-stranded DNA virus, allows substitution of large pieces of adenoviral DNA with foreign sequences up to 7 kB. In contrast to retrovirus, the adenoviral infection of host cells does not result in chromosomal integration because adenoviral DNA can replicate in an episomal manner without potential genotoxicity. Also, adenoviruses are structurally stable, and no genome rearrangement has been detected after extensive amplification. Adenovirus can infect virtually all epithelial cells regardless of their cell cycle stage. So far, adenoviral infection appears to be linked only to mild disease such as acute respiratory disease in humans.

Adenovirus is particularly suitable for use as a gene transfer vector because of its mid-sized genome, ease of manipulation, high titer, wide target cell range and high infectivity. Both ends of the viral genome contain 100-200 base pair inverted repeats (ITRs), which are cis elements necessary for viral DNA replication and packaging. The early (E) and late (L) regions of the genome contain different transcription units that are divided by the onset of viral DNA replication. The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression and host cell shut-off. The products of the late genes, including the majority of the viral capsid proteins, are expressed only after significant processing of a single primary transcript issued by the major late promoter (MLP). The MLP, (located at 16.8 m.u.) is particularly efficient during the late phase of infection, and all the mRNAs issued from this promoter possess a 5′-tripartite leader (TPL) sequence which makes them preferred mRNA's for translation.

In one system, recombinant adenovirus is generated from homologous recombination between shuttle vector and provirus vector. Due to the possible recombination between two proviral vectors, wild-type adenovirus may be generated from this process. Therefore, it is critical to isolate a single clone of virus from an individual plaque and examine its genomic structure.

Generation and propagation of the current adenovirus vectors, which are replication deficient, depend on a unique helper cell line, designated 293, which was transformed from human embryonic kidney cells by Ad5 DNA fragments and constitutively expresses E1 proteins. Since the E3 region is dispensable from the adenovirus genome, the current adenovirus vectors, with the help of 293 cells, carry foreign DNA in either the E1, the D3 or both regions. In nature, adenovirus can package approximately 105% of the wild-type genome, providing capacity for about 2 extra kb of DNA. Combined with the approximately 5.5 kb of DNA that is replaceable in the E1 and E3 regions, the maximum capacity of the current adenovirus vector is under 7.5 kb, or about 15% of the total length of the vector. More than 80% of the adenovirus viral genome remains in the vector backbone and is the source of vector-borne cytotoxicity. Also, the replication deficiency of the E1-deleted virus is incomplete.

Helper cell lines may be derived from human cells such as human embryonic kidney cells, muscle cells, hematopoietic cells or other human embryonic mesenchymal or epithelial cells. Alternatively, the helper cells may be derived from the cells of other mammalian species that are permissive for human adenovirus. Such cells include, e.g., Vero cells or other monkey embryonic mesenchymal or epithelial cells. As stated above, the preferred helper cell line is 293.

The adenovirus may be of any of the 42 different known serotypes or subgroups A-F. Adenovirus type 5 of subgroup C is the preferred starting material in order to obtain the conditional replication-defective adenovirus vector for use as described herein. This is because Adenovirus type 5 is a human adenovirus about which a great deal of biochemical and genetic information is known, and it has historically been used for most constructions employing adenovirus as a vector.

As stated above, the typical vector according to the present disclosure is replication defective and will not have an adenovirus E1 region. Thus, it will be most convenient to introduce the polynucleotide encoding the gene of interest at the position from which the E1-coding sequences have been removed. However, the position of insertion of the construct within the adenovirus sequences is not critical. The polynucleotide encoding the gene of interest may also be inserted in lieu of the deleted E3 region in E3 replacement vectors, or in the E4 region where a helper cell line or helper virus complements the E4 defect.

Adenovirus is easy to grow and manipulate and exhibits broad host range in vitro and in vivo. This group of viruses can be obtained in high titers, e.g., 10⁹-10¹² plaque-forming units per ml, and they are highly infective. The life cycle of adenovirus does not require integration into the host cell genome. The foreign genes delivered by adenovirus vectors are episomal and, therefore, have low genotoxicity to host cells. No side effects have been reported in studies of vaccination with wild-type adenovirus, demonstrating their safety and therapeutic potential as in vivo gene transfer vectors.

Retroviruses. The retroviruses are a group of single-stranded RNA viruses characterized by an ability to convert their RNA to double-stranded DNA in infected cells by a process of reverse-transcription. The resulting DNA then stably integrates into cellular chromosomes as a provirus and directs synthesis of viral proteins. The integration results in the retention of the viral gene sequences in the recipient cell and its descendants. The retroviral genome contains three genes, gag, pol, and env that code for capsid proteins, polymerase enzyme, and envelope components, respectively. A sequence found upstream from the gag gene contains a signal for packaging of the genome into virions. Two long terminal repeat (LTR) sequences are present at the 5′ and 3′ ends of the viral genome.

In order to construct a retroviral vector, a nucleic acid encoding a gene of interest is inserted into the viral genome in the place of certain viral sequences to produce a virus that is replication-defective. In order to produce virions, a packaging cell line containing the gag, pol, and env genes but without the LTR and packaging components is constructed. When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media. The media containing the recombinant retroviruses is then collected, optionally concentrated, and used for gene transfer. Retroviral vectors are able to infect a broad variety of cell types. However, integration and stable expression require the division of host cells.

A novel approach designed to allow specific targeting of retrovirus vectors was recently developed based on the chemical modification of a retrovirus by the chemical addition of lactose residues to the viral envelope. This modification could permit the specific infection of hepatocytes via sialoglycoprotein receptors.

A different approach to targeting of recombinant retroviruses was designed in which biotinylated antibodies against a retroviral envelope protein and against a specific cell receptor were used. The antibodies are coupled via the biotin components by using streptavidin. Using antibodies against major histocompatibility complex class I and class II antigens, a variety of human cells that bear those surface antigens may be infected with an ecotropic virus in vitro.

Other viral vectors. Other viral vectors may be employed as expression constructs. For example, vectors derived from viruses such as vaccinia virus and herpesviruses may be employed. They offer several attractive features for various mammalian cells.

Non-viral methods. Several non-viral methods for the transfer of expression constructs into cultured mammalian cells also are contemplated. These include calcium phosphate precipitation, DEAE-dextran, electroporation, direct microinjection, DNA-loaded liposomes and lipofectamine-DNA complexes, cell sonication, gene bombardment using high velocity microprojectiles, and receptor-mediated transfection. Some of these techniques may be successfully adapted for in vivo or ex vivo use.

Once the expression construct has been delivered into the cell the nucleic acid encoding the gene of interest may be positioned and expressed at different sites. In certain embodiments, the nucleic acid encoding the gene may be stably integrated into the genome of the cell. This integration may be in the cognate location and orientation via homologous recombination (gene replacement) or it may be integrated in a random, non-specific location (gene augmentation). In yet further embodiments, the nucleic acid may be stably maintained in the cell as a separate, episomal segment of DNA. Such nucleic acid segments or “episomes” encode sequences sufficient to permit maintenance and replication independent of or in synchronization with the host cell cycle. How the expression construct is delivered to a cell and where in the cell the nucleic acid remains is dependent on the type of expression construct employed.

In yet another embodiment, the expression construct may simply consist of naked recombinant DNA or plasmids. Transfer of the construct may be performed by any of the methods mentioned above which physically or chemically permeabilize the cell membrane. This is particularly applicable for transfer in vitro but it may be applied to in vivo use as well. Polyomavirus DNA has been successfully injected in the form of calcium phosphate precipitates into liver and spleen of adult and newborn mice demonstrating active viral replication and acute infection. Direct intraperitoneal injection of calcium phosphate-precipitated plasmids, resulting in expression of the transfected genes, may also be used. It is envisioned that DNA encoding a gene of interest may also be transferred in a similar manner in vivo and express the gene product.

In still another embodiment for transferring a naked DNA expression construct into cells may involve particle bombardment. This method depends on the ability to accelerate DNA-coated microprojectiles to a high velocity allowing them to pierce cell membranes and enter cells without killing them. Several devices for accelerating small particles have been developed. One such device relies on a high voltage discharge to generate an electrical current, which in turn provides the motive force. The microprojectiles used have consisted of biologically inert substances such as tungsten or gold beads.

Selected organs including the liver, skin, and muscle tissue of rats and mice have been bombarded in vivo. This may require surgical exposure of the tissue or cells, to eliminate any intervening tissue between the gun and the target organ, i.e., ex vivo treatment. Again, DNA encoding a particular gene may be delivered via this method and still be incorporated by the instant disclosure.

In a further embodiment, the expression construct may be entrapped in a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers. Also contemplated are lipofectamine-DNA complexes.

Liposome-mediated nucleic acid delivery and expression of foreign DNA in vitro has been very successful. A reagent known as Lipofectamine 2000™ is widely used and commercially available.

In certain embodiments, the liposome may be complexed with a hemagglutinating virus (HVJ). This has been shown to facilitate fusion with the cell membrane and promote cell entry of liposome-encapsulated DNA. In other embodiments, the liposome may be complexed or employed in conjunction with nuclear non-histone chromosomal proteins (HMG-1). In yet further embodiments, the liposome may be complexed or employed in conjunction with both HVJ and HMG-1. In that such expression constructs have been successfully employed in transfer and expression of nucleic acid in vitro and in vivo, then they are applicable. Where a bacterial promoter is employed in the DNA construct, it also will be desirable to include within the liposome an appropriate bacterial polymerase.

Other expression constructs which can be employed to deliver a nucleic acid encoding a particular gene into cells are receptor-mediated delivery vehicles. These take advantage of the selective uptake of macromolecules by receptor-mediated endocytosis in almost all eukaryotic cells. Because of the cell type-specific distribution of various receptors, the delivery can be highly specific.

Receptor-mediated gene targeting vehicles generally consist of two components: a cell receptor-specific ligand and a DNA-binding agent. Several ligands have been used for receptor-mediated gene transfer. The most extensively characterized ligands are asialoorosomucoid (ASOR) and transferrin. A synthetic neoglycoprotein, which recognizes the same receptor as ASOR, may be used as a gene delivery vehicle. In some embodiments, epidermal growth factor (EGF) may be used to deliver genes.

### III. METHODS OF MAKING TRANSGENIC MICE

A particular embodiment provides transgenic animals that contain mutations in the dystrophin gene. Also, transgenic animals may express a marker that reflects the production of mutant or normal dystrophin gene product.

In a general aspect, a transgenic animal is produced by the integration of a given construct into the genome in a manner that permits the expression of the transgene using methods discussed above. Methods for producing transgenic animals are generally described by Wagner and Hoppe (U.S. Pat. No. 4,873,191; incorporated herein by reference), and Brinster et al. (1985; incorporated herein by reference).

Typically, the construct is transferred by microinjection into a fertilized egg. The microinjected eggs are implanted into a host female, and the progeny are screened for the expression of the transgene. Transgenic animals may be produced from the fertilized eggs from a number of animals including, but not limited to reptiles, amphibians, birds, mammals, and fish.

RNA for microinjection can be prepared by any means known in the art. For example, RNA for microinjection can be cleaved with enzymes appropriate for removing the bacterial plasmid sequences, and the RNA fragments electrophoresed on 1% agarose gels in TBE buffer, using standard techniques. The RNA bands are visualized by staining with ethidium bromide, and the band containing the expression sequences is excised. The excised band is then placed in dialysis bags containing 0.3 M sodium acetate, pH 7.0. RNA is electroeluted into the dialysis bags, extracted with a 1:1 phenol:chloroform solution and precipitated by two volumes of ethanol. The RNA is redissolved in 1 ml of low salt buffer (0.2 M NaCl, 20 mM Tris, pH 7.4, and 1 mM EDTA) and purified on an Elutip-D® column. The column is first primed with 3 ml of high salt buffer (1 M NaCl, 20 mM Tris, pH 7.4, and 1 mM EDTA) followed by washing with 5 ml of low salt buffer. The DNA solutions are passed through the column three times to bind RNA to the column matrix. After one wash with 3 ml of low salt buffer, the RNA is eluted with 0.4 ml high salt buffer and precipitated by two volumes of ethanol. RNA concentrations are measured by absorption at 260 nm in a UV spectrophotometer. For microinjection, DNA concentrations are adjusted to 3 μg/ml in 5 mM Tris, pH 7.4 and 0.1 mM EDTA.

In an exemplary microinjection procedure, female mice six weeks of age are induced to superovulate with a 5 IU injection (0.1 cc, ip) of pregnant mare serum gonadotropin (PMSG; Sigma) followed 48 hours later by a 5 IU injection (0.1 cc, ip) of human chorionic gonadotropin (hCG; Sigma). Females are placed with males immediately after hCG injection. Twenty-one hours after hCG injection, the mated females are sacrificed by CO2 asphyxiation or cervical dislocation and embryos are recovered from excised oviducts and placed in Dulbecco's phosphate buffered saline with 0.5% bovine serum albumin (BSA; Sigma). Surrounding cumulus cells are removed with hyaluronidase (1 mg/ml). Pronuclear embryos are then washed and placed in Earle's balanced salt solution containing 0.5% BSA (EBSS) in a 37.5° C. incubator with a humidified atmosphere at 5% CO₂, 95% air until the time of injection. Embryos can be implanted at the two-cell stage.

Randomly cycling adult female mice are paired with vasectomized males. C57BL/6 or Swiss mice or other comparable strains can be used for this purpose. Recipient females are mated at the same time as donor females. At the time of embryo transfer, the recipient females are anesthetized with an intraperitoneal injection of 0.015 ml of 2.5% avertin per gram of body weight. The oviducts are exposed by a single midline dorsal incision. An incision is then made through the body wall directly over the oviduct. The ovarian bursa is then torn with watchmakers forceps. Embryos to be transferred are placed in DPBS (Dulbecco's phosphate buffered saline) and in the tip of a transfer pipet (about 10 to 12 embryos). The pipet tip is inserted into the infundibulum and the embryos transferred. After the transfer, the incision is closed by two sutures.

### IV. PHARMACEUTICAL COMPOSITIONS AND DELIVERY METHODS

Where clinical applications are contemplated, pharmaceutical compositions will be prepared in a form appropriate for the intended application. Generally, this will entail preparing compositions that are essentially free of pyrogens, as well as other impurities that could be harmful to humans or animals.

One will generally desire to employ appropriate salts and buffers to render drugs, proteins or delivery vectors stable and allow for uptake by target cells. Aqueous compositions of the disclosure may comprise an effective amount of the drug, vector or proteins, dissolved or dispersed in a pharmaceutically acceptable carrier or aqueous medium. The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes solvents, buffers, solutions, dispersion media, coatings, antibacterial and antifungal agents, isotonic and absorption delaying agents and the like acceptable for use in formulating pharmaceuticals, such as pharmaceuticals suitable for administration to humans. The use of such media and agents for pharmaceutically active substances is well known in the art. Except insofar as any conventional media or agent is incompatible with the active ingredients of the present disclosure, its use in therapeutic compositions is contemplated. Supplementary active ingredients also can be incorporated into the compositions, provided they do not inactivate the vectors or cells of the compositions.

The active compositions of the present disclosure may include classic pharmaceutical preparations. Administration of these compositions according to the present disclosure may be via any common route so long as the target tissue is available via that route, but generally including systemic administration. This includes oral, nasal, or buccal. Alternatively, administration may be by intradermal, subcutaneous, intramuscular, intraperitoneal or intravenous injection, or by direct injection into muscle tissue. Such compositions would normally be administered as pharmaceutically acceptable compositions, as described supra.

The active compounds may also be administered parenterally or intraperitoneally. By way of illustration, solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations generally contain a preservative to prevent the growth of microorganisms.

The pharmaceutical forms suitable for injectable use include, for example, sterile aqueous solutions or dispersions and sterile powders for the extemporaneous preparation of sterile injectable solutions or dispersions. Generally, these preparations are sterile and fluid to the extent that easy injectability exists. Preparations should be stable under the conditions of manufacture and storage and should be preserved against the contaminating action of microorganisms, such as bacteria and fungi. Appropriate solvents or dispersion media may contain, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions may be prepared by incorporating the active compounds in an appropriate amount into a solvent along with any other ingredients (for example as enumerated above) as desired, followed by filtered sterilization. Generally, dispersions are prepared by incorporating the various sterilized active ingredients into a sterile vehicle which contains the basic dispersion medium and the desired other ingredients, e.g., as enumerated above. In the case of sterile powders for the preparation of sterile injectable solutions, the preferred methods of preparation include vacuum-drying and freeze-drying techniques which yield a powder of the active ingredient(s) plus any additional desired ingredient from a previously sterile-filtered solution thereof.

The compositions generally may be formulated in a neutral or salt form. Pharmaceutically-acceptable salts include, for example, acid addition salts (formed with the free amino groups of the protein) derived from inorganic acids (e.g., hydrochloric or phosphoric acids, or from organic acids (e.g., acetic, oxalic, tartaric, mandelic, and the like). Salts formed with the free carboxyl groups of the protein can also be derived from inorganic bases (e.g., sodium, potassium, ammonium, calcium, or ferric hydroxides) or from organic bases (e.g., isopropylamine, trimethylamine, histidine, procaine and the like.

Upon formulation, solutions are preferably administered in a manner compatible with the dosage formulation and in such amount as is therapeutically effective. The formulations may easily be administered in a variety of dosage forms such as injectable solutions, drug release capsules and the like. For parenteral administration in an aqueous solution, for example, the solution generally is suitably buffered, and the liquid diluent first rendered isotonic for example with sufficient saline or glucose. Such aqueous solutions may be used, for example, for intravenous, intramuscular, subcutaneous and intraperitoneal administration. Preferably, sterile aqueous media are employed as is known to those of skill in the art, particularly in light of the present disclosure. By way of illustration, a single dose may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

### V. DMD SUBJECT CHARACTERISTICS AND CLINICAL PRESENTATION

Duchenne muscular dystrophy (DMD) is a recessive X-linked form of muscular dystrophy, affecting around 1 in 5000 boys, which results in muscle degeneration and premature death. The disorder is caused by a mutation in the gene dystrophin, located on the human X chromosome, which codes for the protein dystrophin. Dystrophin is an important component within muscle tissue that provides structural stability to the dystroglycan complex (DGC) of the cell membrane. While both sexes can carry the mutation, females are rarely affected with the skeletal muscle form of the disease.

Mutations vary in nature and frequency. Large genetic deletions are found in about 60-70% of cases, large duplications are found in about 10% of cases, and point mutants or other small changes account for about 15-30% of cases. An examination of some 7000 mutations catalogued a total of 5,682 large mutations (80% of total mutations), of which 4,894 (86%) were deletions (1 exon or larger) and 784 (14%) were duplications (1 exon or larger). There were 1,445 small mutations (smaller than 1 exon, 20% of all mutations), of which 358 (25%) were small deletions and 132 (9%) small insertions, while 199 (14%) affected the splice sites. Point mutations totaled 756 (52% of small mutations) with 726 (50%) nonsense mutations and 30 (2%) missense mutations. Finally, 22 (0.3%) mid-intronic mutations were observed. In addition, mutations were identified within the database that would potentially benefit from novel genetic therapies for DMD including stop codon read-through therapies (10% of total mutations) and exon skipping therapy (80% of deletions and 55% of total mutations).

A. Symptoms

Symptoms usually appear in boys between the ages of 2 and 3 and may be visible in early infancy. Even though symptoms do not appear until early infancy, laboratory testing can identify children who carry the active mutation at birth. Progressive proximal muscle weakness of the legs and pelvis associated with loss of muscle mass is observed first. Eventually this weakness spreads to the arms, neck, and other areas. Early signs may include pseudohypertrophy (enlargement of calf and deltoid muscles), low endurance, and difficulties in standing unaided or inability to ascend staircases. As the condition progresses, muscle tissue experiences wasting and is eventually replaced by fat and fibrotic tissue (fibrosis). By age 10, braces may be required to aid in walking but most patients are wheelchair dependent by age 12. Later symptoms may include abnormal bone development that lead to skeletal deformities, including curvature of the spine. Due to progressive deterioration of muscle, loss of movement occurs, eventually leading to paralysis. Intellectual impairment may or may not be present but if present, does not progressively worsen as the child ages. The average life expectancy for males afflicted with DMD is around 25.

The main symptom of Duchenne muscular dystrophy, a progressive neuromuscular disorder, is muscle weakness associated with muscle wasting with the voluntary muscles being first affected, especially those of the hips, pelvic area, thighs, shoulders, and calves. Muscle weakness also occurs later, in the arms, neck, and other areas. Calves are often enlarged. Symptoms usually appear before age 6 and may appear in early infancy. Other physical symptoms are:
Awkward manner of walking, stepping, or running—(patients tend to walk on their forefeet, because of an increased calf muscle tone. Also, toe walking is a compensatory adaptation to knee extensor weakness.)
Frequent falls
Fatigue
Difficulty with motor skills (running, hopping, jumping)
Lumbar hyperlordosis, possibly leading to shortening of the hip-flexor muscles. This has an effect on overall posture and a manner of walking, stepping, or running.
Muscle contractures of Achilles tendon and hamstrings impair functionality because the muscle fibers shorten and fibrose in connective tissue
Progressive difficulty walking
Muscle fiber deformities
Pseudohypertrophy (enlarging) of tongue and calf muscles. The muscle tissue is eventually replaced by fat and connective tissue, hence the term pseudohypertrophy.
Higher risk of neurobehavioral disorders (e.g., ADHD), learning disorders (dyslexia), and non-progressive weaknesses in specific cognitive skills (in particular short-term verbal memory), which are believed to be the result of absent or dysfunctional dystrophin in the brain.
Eventual loss of ability to walk (usually by the age of 12)
Skeletal deformities (including scoliosis in some cases)
Trouble getting up from lying or sitting position
   The condition can often be observed clinically from the moment the patient takes his first steps, and the ability to walk usually completely disintegrates between the time the boy is 9 to 12 years of age. Most men affected with DMD become essentially “paralyzed from the neck down” by the age of 21. Muscle wasting begins in the legs and pelvis, then progresses to the muscles of the shoulders and neck, followed by loss of arm muscles and respiratory muscles. Calf muscle enlargement (pseudohypertrophy) is quite obvious. Cardiomyopathy particularly (dilated cardiomyopathy) is common, but the development of congestive heart failure or arrhythmia (irregular heartbeat) is only occasional.

A positive Gowers' sign reflects the more severe impairment of the lower extremities muscles. The child helps himself to get up with upper extremities: first by rising to stand on his arms and knees, and then “walking” his hands up his legs to stand upright. Affected children usually tire more easily and have less overall strength than their peers. Creatine kinase (CPK-MM) levels in the bloodstream are extremely high. An electromyography (EMG) shows that weakness is caused by destruction of muscle tissue rather than by damage to nerves. Genetic testing can reveal genetic errors in the Xp21 gene. A muscle biopsy (immunohistochemistry or immunoblotting) or genetic test (blood test) confirms the absence of dystrophin, although improvements in genetic testing often make this unnecessary.

Additional symptoms may include:
Abnormal heart muscle (cardiomyopathy)
Congestive heart failure or irregular heart rhythm (arrhythmia)
Deformities of the chest and back (scoliosis)
Enlarged muscles of the calves, buttocks, and shoulders (around age 4 or 5). These muscles are eventually replaced by fat and connective tissue (pseudohypertrophy).
Loss of muscle mass (atrophy)
Muscle contractures in the heels, legs
Muscle deformities
Respiratory disorders, including pneumonia and swallowing with food or fluid passing into the lungs (in late stages of the disease)

B. Causes

Duchenne muscular dystrophy (DMD) is caused by a mutation of the dystrophin gene at locus Xp21, located on the short arm of the X chromosome. Dystrophin is responsible for connecting the cytoskeleton of each muscle fiber to the underlying basal lamina (extracellular matrix), through a protein complex containing many subunits. The absence of dystrophin permits excess calcium to penetrate the sarcolemma (the cell membrane). Alterations in calcium and signaling pathways cause water to enter into the mitochondria, which then burst.

In skeletal muscle dystrophy, mitochondrial dysfunction gives rise to an amplification of stress-induced cytosolic calcium signals and an amplification of stress-induced reactive-oxygen species (ROS) production. In a complex cascading process that involves several pathways and is not clearly understood, increased oxidative stress within the cell damages the sarcolemma and eventually results in the death of the cell. Muscle fibers undergo necrosis and are ultimately replaced with adipose and connective tissue.

DMD is inherited in an X-linked recessive pattern. Females will typically be carriers for the disease while males will be affected. Typically, a female carrier will be unaware they carry a mutation until they have an affected son. The son of a carrier mother has a 50% chance of inheriting the defective gene from his mother. The daughter of a carrier mother has a 50% chance of being a carrier and a 50% chance of having two normal copies of the gene. In all cases, an unaffected father will either pass a normal Y to his son or a normal X to his daughter. Female carriers of an X-linked recessive condition, such as DMD, can show symptoms depending on their pattern of X-inactivation.

Duchenne muscular dystrophy has an incidence of 1 in 5,000 male infants. Mutations within the dystrophin gene can either be inherited or occur spontaneously during germline transmission.

C. Diagnosis

Genetic counseling is advised for people with a family history of the disorder. Duchenne muscular dystrophy can be detected with about 95% accuracy by genetic studies.

DNA test. The muscle-specific isoform of the dystrophin gene is composed of 79 exons, and DNA testing and analysis can usually identify the specific type of mutation of the exon or exons that are affected. DNA testing confirms the diagnosis in most cases.

Muscle biopsy. If DNA testing fails to find the mutation, a muscle biopsy test may be performed. A small sample of muscle tissue is extracted (usually with a scalpel instead of a needle) and a dye is applied that reveals the presence of dystrophin. Complete absence of the protein indicates the condition.

Over the past several years DNA tests have been developed that detect more of the many mutations that cause the condition, and muscle biopsy is not required as often to confirm the presence of Duchenne's.

Prenatal tests. DMD is carried by an X-linked recessive gene. Males have only one X chromosome, so one copy of the mutated gene will cause DMD. Fathers cannot pass X-linked traits on to their sons, so the mutation is transmitted by the mother.

If the mother is a carrier, and therefore one of her two X chromosomes has a DMD mutation, there is a 50% chance that a female child will inherit that mutation as one of her two X chromosomes, and be a carrier. There is a 50% chance that a male child will inherit that mutation as his one X chromosome, and therefore have DMD.

Prenatal tests can tell whether their unborn child has the most common mutations. There are many mutations responsible for DMD, and some have not been identified, so genetic testing only works when family members with DMD have a mutation that has been identified.

Prior to invasive testing, determination of the fetal sex is important; while males are sometimes affected by this X-linked disease, female DMD is extremely rare. This can be achieved by ultrasound scan at 16 weeks or more recently by free fetal DNA testing. Chorion villus sampling (CVS) can be done at 11-14 weeks, and has a 1% risk of miscarriage. Amniocentesis can be done after 15 weeks, and has a 0.5% risk of miscarriage. Fetal blood sampling can be done at about 18 weeks. Another option in the case of unclear genetic test results is fetal muscle biopsy.

D. Treatment

There is no current cure for DMD, and an ongoing medical need has been recognized by regulatory authorities. Phase 1-2a trials with exon skipping treatment for certain mutations have halted decline and produced clinical improvements in walking. Sarepta's drug Exondys 51 (eteplirsen) has recently received FDA approval. However, treatment is generally aimed at controlling the onset of symptoms to maximize the quality of life, and include the following:
Corticosteroids such as prednisolone and deflazacort increase energy and strength and defer severity of some symptoms.
Randomized control trials have shown that beta-2-agonists increase muscle strength but do not modify disease progression. Follow-up time for most RCTs on beta2-agonists is only around 12 months and hence results cannot be extrapolated beyond that time frame.
Mild, non jarring physical activity such as swimming is encouraged. Inactivity (such as bed rest) can worsen the muscle disease.
Physical therapy is helpful to maintain muscle strength, flexibility, and function.
Orthopedic appliances (such as braces and wheelchairs) may improve mobility and the ability for self-care. Form-fitting removable leg braces that hold the ankle in place during sleep can defer the onset of contractures.
Appropriate respiratory support as the disease progresses is important.
   Comprehensive multi-disciplinary care standards/guidelines for DMD have been developed by the Centers for Disease Control and Prevention (CDC), and and are available at treat-nmd.eu/dmd/care/diagnosis-management-DMD.

1. Physical Therapy

Physical therapists are concerned with enabling patients to reach their maximum physical potential. Their aim is to:
minimize the development of contractures and deformity by developing a programme of stretches and exercises where appropriate
anticipate and minimize other secondary complications of a physical nature by recommending bracing and durable medical equipment
monitor respiratory function and advise on techniques to assist with breathing exercises and methods of clearing secretions

2. Respiration Assistance

Modern “volume ventilators/respirators,” which deliver an adjustable volume (amount) of air to the person with each breath, are valuable in the treatment of people with muscular dystrophy related respiratory problems. The ventilator may require an invasive endotracheal or tracheotomy tube through which air is directly delivered, but, for some people non-invasive delivery through a face mask or mouthpiece is sufficient. Positive airway pressure machines, particularly bi-level ones, are sometimes used in this latter way. The respiratory equipment may easily fit on a ventilator tray on the bottom or back of a power wheelchair with an external battery for portability.

Ventilator treatment may start in the mid to late teens when the respiratory muscles can begin to collapse. If the vital capacity has dropped below 40% of normal, a volume ventilator/respirator may be used during sleeping hours, a time when the person is most likely to be under ventilating (“hypoventilating”). Hypoventilation during sleep is determined by a thorough history of sleep disorder with an oximetry study and a capillary blood gas (See Pulmonary Function Testing). A cough assist device can help with excess mucus in lungs by hyperinflation of the lungs with positive air pressure, then negative pressure to get the mucus up. If the vital capacity continues to decline to less than 30 percent of normal, a volume ventilator/respirator may also be needed during the day for more assistance. The person gradually will increase the amount of time using the ventilator/respirator during the day as needed.

E. Prognosis

Duchenne muscular dystrophy is a progressive disease which eventually affects all voluntary muscles and involves the heart and breathing muscles in later stages. The life expectancy is currently estimated to be around 25, but this varies from patient to patient. Recent advancements in medicine are extending the lives of those afflicted. The Muscular Dystrophy Campaign, which is a leading UK charity focusing on all muscle disease, states that “with high standards of medical care young men with Duchenne muscular dystrophy are often living well into their 30s.”

In rare cases, persons with DMD have been seen to survive into the forties or early fifties, with the use of proper positioning in wheelchairs and beds, ventilator support (via tracheostomy or mouthpiece), airway clearance, and heart medications, if required. Early planning of the required supports for later-life care has shown greater longevity in people living with DMD.

Curiously, in the mdx mouse model of Duchenne muscular dystrophy, the lack of dystrophin is associated with increased calcium levels and skeletal muscle myonecrosis. The intrinsic laryngeal muscles (ILM) are protected and do not undergo myonecrosis. ILM have a calcium regulation system profile suggestive of a better ability to handle calcium changes in comparison to other muscles, and this may provide a mechanistic insight for their unique pathophysiological properties. The ILM may facilitate the development of novel strategies for the prevention and treatment of muscle wasting in a variety of clinical scenarios.

### VI. SEQUENCE TABLES

TABLE 8
Sequence of primers for generating sgRNA targeting
human Dmd exon 44, exon 46, and exon 53

| ID | Sequence (5′-3′) | SEQ ID NO. |
|---|---|---|
| hDMD-E43g1-top | CACCGTTTTAAAATTTTTATATTA | 340 |
| hDMD-E43g1-bottom | aaacTAATATAAAAATTTTAAAAC | 341 |
| hDMD-E43g2-top | CACCGTTTTTATATTACAGAATATAA | 342 |
| hDMD-E43g2-bottom | aaacTTATATTCTGTAATATAAAAA | 343 |
| hDMD-E43g3-top | CACCGATATTACAGAATATAAAAGA | 344 |
| hDMD-E43g3-bottom | aaacTCTTTTATATTCTGTAATAT | 345 |
| hDMD-E43g5-top | CACCGAAATGTACAAGGACCGACAA | 346 |
| hDMD-E43g5-bottom | aaacTTGTCGGTCCTTGTACATTTC | 347 |
| hDMD-E43g4-top | CACCGTATGTGTTACCTACCCTTGT | 348 |
| hDMD-E43g4-bottom | aaacACAAGGGTAGGTAACACATA | 349 |
| hDMD-E43g6-top | CACCGTACAAGGACCGACAAGGGT | 350 |
| hDMD-E43g6-bottom | aaacACCCTTGTCGGTCCTTGTAC | 351 |
| hDMD-E44g1-top | CACCGATCCATATGCTTTTACCTGC | 352 |
| hDMD-E44g1-bottom | aaacGCAGGTAAAAGCATATGGAT | 353 |
| hDMD-E44g2-top | CACCgatccatatgcttttACCTG | 354 |
| hDMD-E44g2-bottom | aaacCAGGTAAAAGCATATGGATC | 355 |
| hDMD-E44g3-top | CACCGCAGATCTGTCAAATCGCCTG | 356 |
| hDMD-E44g3-bottom | aaacCAGGCGATTTGACAGATCTG | 357 |
| hDMD-E44g4-top | CACCGTAAATACAAATGGTATCTTA | 358 |
| hDMD-E44g4-bottom | aaacTAAGATACCATTTGTATTTA | 359 |
| hDMD-E44g5-bottom | aaacGCAGGCGATTTGACAGATCTC | 1 |
| hDMD-E44g5-top | CACCGAGATCTGTCAAATCGCCTGC | 2 |
| hDMD-E44g6-top | CACCGACAGATCTGTTGAGAAATGG | 3 |
| hDMD-E44g6-bottom | aaacCCATTTCTCAACAGATCTGTC | 4 |
| hDMD-E44g7-bottom | aaacGAGAATTGGGAACATGCTAAC | 5 |
| hDMD-E44g7-top | CACCGTTAGCATGTTCCCAATTCTC | 6 |
| hDMD-E44g8-bottom | aaacTTTGTATTTAGCATGTTCCC | 7 |
| hDMD-E44g8-top | CACCGGGAACATGCTAAATACAAA | 8 |
| hDMD-E44g9-top | CACCGTTGACAGATCTGTTGAGAAA | 9 |
| hDMD-E44g9-bottom | aaacTTTCTCAACAGATCTGTCAAC | 10 |
| hDMD-E44g10-bottom | aaacATTCTCAGGAATTTGTGTCTC | 11 |
| hDMD-E44g10-top | CACCGAGACACAAATTCCTGAGAAT | 12 |
| hDMD-E44g11-bottom | aaacAATTCTCAGGAATTTGTGTC | 13 |
| hDMD-E44g11-top | CACCGACACAAATTCCTGAGAATT | 14 |
| hDMD-E44g12-bottom | aaactatgcttttACCTGCAGGCGC | 360 |
| hDMD-E44g12-top | CACCGCGCCTGCAGGTaaaagcata | 361 |
| hDMD-E44g13-top | CACCGtttACCTGCAGGCGATTTGA | 362 |
| hDMD-E44g13-bottom | aaacTCAAATCGCCTGCAGGTaaaC | 363 |
| hDMD-E44g14-bottom | aaacAACAGATCTGTCAAATCGCC | 364 |
| hDMD-E44g14-top | CACCGGCGATTTGACAGATCTGTT | 365 |
| hDMD-E44g15-bottom | aaacCTGTTAGCCACTGATTAAATC | 366 |
| hDMD-E44g15-top | CACCGATTTAATCAGTGGCTAACAG | 367 |
| hDMD-E44g16-bottom | aaacACAGAAGCTGAACAGTTTCTC | 368 |
| hDMD-E44g16-top | CACCGAGAAACTGTTCAGCTTCTGT | 369 |
| hDMD-E44g17-bottom | aaacTTCAGCTTCTGTTAGCCACTC | 370 |
| hDMD-E44g17-top | CACCGAGTGGCTAACAGAAGCTGAA | 371 |
| hDMD-E44g18-bottom | aaacTCTGAGAAACTGTTCAGCTTC | 372 |
| hDMD-E44g18-top | CACCGAAGCTGAACAGTTTCTCAGA | 373 |
| hDMD-E44g19-bottom | aaacAGAATTGGGAACATGCTAAAC | 374 |
| hDMD-E44g19-top | CACCGTTTAGCATGTTCCCAATTCT | 375 |
| hDMD-E44g20-bottom | aaacAATACAAATGGTATCTTAAGC | 376 |
| hDMD-E44g20-top | CACCGCTTAAGATACCATTTGTATT | 377 |
| hDMD-E44g21-bottom | aaacAAGATACCATTTGTATTTAGC | 378 |
| hDMD-E44g21-top | CACCGCTAAATACAAATGGTATCTT | 379 |
| hDMD-E44g22-bottom | aaacACCTTAAGATACCATTTGTAC | 380 |
| hDMD-E44g22-top | CACCGTACAAATGGTATCTTAAGGT | 381 |
| hDMD-E44g23-bottom | aaacAAGGTAAGTCTTTGATTTGTC | 382 |
| hDMD-E44g23-top | CACCGACAAATCAAAGACTTACCTT | 383 |
| hDMD-E46g1-top | CACCGttattcttctttctccagGC | 384 |
| hDMD-E46g1-bottom | aaacGCCTGGAGAAAGAAGAATAA | 385 |
| hDMD-E46g2-top | CACCGAATTTTATTCTTCTTTCTCC | 386 |
| hDMD-E46g2-bottom | aaacGGAGAAAGAAGAATAAAATT | 387 |
| hDMD-E46g5-bottom | aaacGGCTAGAAGAACAAAAGAATC | 29 |
| hDMD-E46g5-top | CACCGATTCTTTTGTTCTTCTAGCC | 30 |
| hDMD-E46g6-bottom | aaacACCATAAAACAAATTCATTTC | 31 |
| hDMD-E46g6-top | CACCGAAATGAATTTGTTTTATGGT | 32 |
| hDMD-E46g7-top | CACCGTGAATTTGTTTTATGGTTGG | 33 |
| hDMD-E46g7-bottom | aaacCCAACCATAAAACAAATTCAC | 34 |
| hDMD-E46g8-bottom | aaacTGACTTGCTCAAGCTTTTCTC | 35 |
| hDMD-E46g8-top | CACCGAGAAAAGCTTGAGCAAGTCA | 36 |
| hDMD-E46g9-top | CACCGTTCTTCTAGCCTGGAGAAAG | 388 |
| hDMD-E46g9-bottom | aaacCTTTCTCCAGGCTAGAAGAAC | 389 |
| hDMD-E46g10-bottom | aaacGAGAAAGAAGAATAAAATTGC | 390 |
| hDMD-E46g10-top | CACCGCAATTTTATTCTTCTTTCTC | 391 |
| hDMD-E46g11-bottom | aaacTCTCCAGGCTAGAAGAACAAC | 392 |
| hDMD-E46g11-top | CACCGTTGTTCTTCTAGCCTGGAGA | 393 |
| hDMD-E46g12-bottom | aaacCAGGCTAGAAGAACAAAAGAC | 394 |
| hDMD-E46g12-top | CACCGTCTTTTGTTCTTCTAGCCTG | 395 |
| hDMD-E46g13-bottom | aaacCTAGCCTGGAGAAAGAAGAAC | 396 |
| hDMD-E46g13-top | CACCGTTCTTCTTTCTCCAGGCTAG | 397 |
| hDMD-E46g14-bottom | aaacAAAGAAAAGCTTGAGCAAGTC | 398 |
| hDMD-E46g14-top | CACCGACTTGCTCAAGCTTTTCTTT | 399 |
| hDMD-E46g15-bottom | aaacGCTCAAGCTTTTCTTTTAGTC | 400 |
| hDMD-E46g15-top | CACCGACTAAAAGAAAAGCTTGAGC | 401 |
| hDMD-E46g16-bottom | aaacGAGCAAGTCAAGGTAATTTTC | 402 |
| hDMD-E46g16-top | CACCGAAAATTACCTTGACTTGCTC | 403 |
| hDMD-E46g17-bottom | aaacGACTTGCTCAAGCTTTTCTTC | 404 |
| hDMD-E46g17-top | CACCGAAGAAAAGCTTGAGCAAGTC | 405 |
| hDMD-E46g18-top | CACCGTCTCCAGGCTAGAAGAACAA | 406 |
| hDMD-E46g18-bottom | aaacTTGTTCTTCTAGCCTGGAGA | 407 |
| hDMD-E46g19-top | CACCGAGAACAAAAGAATATCTTGT | 408 |
| hDMD-E46g19-bottom | aaacACAAGATATTCTTTTGTTCT | 409 |
| hDMD-E46g20-top | CACCGTATCTTGTCAGAATTTCAAA | 410 |
| hDMD-E46g20-bottom | aaacTTTGAAATTCTGACAAGATA | 411 |
| hDMD-E50g1-top | CACCGTGTATGCTTTTCTGTTAAAG | 412 |
| hDMD-E50g1-bottom | aaacCTTTAACAGAAAAGCATACA | 413 |
| hDMD-E50g2-top | CACCGATGTGTAT GCTTTT CT GTTA | 414 |
| hDMD-E50g2-bottom | aaacTAACAGAAAAGCATACACAT | 415 |
| hDMD-E50g3-top | CACCGTGTATGCTTTTCTGTTAAA | 416 |
| hDMD-E50g3-bottom | aaacTTTAACAGAAAAGCATACAC | 417 |
| hDMD-E50g4-top | CACCGATGCTTTTCTGTTAAAGAGG | 418 |
| hDMD-E50g4-bottom | aaacCCTCTTTAACAGAAAAGCAT | 419 |
| hDMD-E50g5-top | CACCGTCTTCTAACTTCCTCTTTAA | 420 |
| hDMD-E50g5-bottom | aaacTTAAAGAGGAAGTTAGAAGA | 421 |
| hDMD-E50g6-top | CACCGTAACTTCCTCTTTAACAGAA | 422 |
| hDMD-E50g6-bottom | aaacTTCTGTTAAAGAGGAAGTTA | 423 |
| hDMD-E50g7-top | CACCGTTTTCTGTTAAAGAGGAAGT | 424 |
| hDMD-E50g7-bottom | aaacACTTCCTCTTTAACAGAAAA | 425 |
| hDMD-E50g8-top | CACCGTCTGTTAAAGAGGAAGTTAG | 426 |
| hDMD-E50g8-bottom | aaacCTAACTTCCTCTTTAACAGA | 427 |
| hDMD-E50g9-top | CACCGAAGAGGAAGTTAGAAGATCT | 428 |
| hDMD-E50g9-bottom | aaacAGATCTTCTAACTTCCTCTT | 429 |
| hDMD-E50g10-top | CACCGAGTTAGAAGATCTGAGCTCT | 430 |
| hDMD-E50g10-bottom | aaacAGAGCTCAGATCTTCTAACT | 431 |
| hDMD-E50g11-top | CACCGTAGAAGATCTGAGCTCTGAG | 432 |
| hDMD-E50g11-bottom | aaacCTCAGAGCTCAGATCTTCTA | 433 |
| hDMD-E50g12-top | CACCGAGATCTGAGCTCTGAGTGGA | 434 |
| hDMD-E50g12-bottom | aaacTCCACTCAGAGCTCAGATCT | 435 |
| hDMD-E50g13-top | CACCGACCGCCTTCCACTCAGAGCT | 436 |
| hDMD-E50g13-bottom | aaacAGCTCTGAGTGGAAGGCGGT | 437 |
| hDMD-E50g14-top | CACCGGTTTACCGCCTTCCACTCA | 438 |
| hDMD-E50g14-bottom | aaacTGAGTGGAAGGCGGTAAACC | 439 |
| hDMD-E50g15-top | CACCGAAGCAGCCTGACCTAGCTCC | 440 |
| hDMD-E50g15-bottom | aaacGGAGCTAGGTCAGGCTGCTT | 441 |
| hDMD-E50g16-top | CACCGTCAGTCCAGGAGCTAGGTC | 442 |
| hDMD-E50g16-bottom | aaacGACCTAGCTCCTGGACTGA | 443 |
| hDMD-E50g17-top | CACCGGTCAGTCCAGGAGCTAGGT | 444 |
| hDMD-E50g17-bottom | aaacACCTAGCTCCTGGACTGACC | 445 |
| hDMD-E50g18-top | CACCGTAGTGGTCAGTCCAGGAGCT | 446 |
| hDMD-E50g18-bottom | aaacAGCTCCTGGACTGACCACTA | 447 |
| hDMD-E50g19-top | CACCGATAGTGGTCAGTCCAGGAGC | 448 |
| hDMD-E50g19-bottom | aaacGCTCCTGGACTGACCACTAT | 449 |
| hDMD-E50g20-top | CACCGTCCAATAGTGGTCAGTCCAG | 450 |
| hDMD-E50g20-bottom | aaacCTGGACTGACCACTATTGGA | 451 |
| hDMD-E50g21-top | CACCGCTCCAATAGTGGTCAGTCC | 452 |
| hDMD-E50g21-bottom | aaacGGACTGACCACTATTGGAGC | 453 |
| hDMD-E50g22-top | CACCGTTACAGGCTCCAATAGTGGT | 454 |
| hDMD-E50g22-bottom | aaacACCACTATTGGAGCCTGTAA | 455 |
| hDMD-E50g23-top | CACCGATACTTACAGGCTCCAATAG | 456 |
| hDMD-E50g23-bottom | aaacCTATTGGAGCCTGTAAGTAT | 457 |
| hDMD-E50g24-top | CACCGAGTATACTTACAGGCTCCAA | 458 |
| hDMD-E50g24-bottom | aaacTTGGAGCCTGTAAGTATACT | 459 |
| hDMD-E50g25-top | CACCGCTCCTGGACTGACCACTAT | 460 |
| hDMD-E50g25-bottom | aaacATAGTGGTCAGTCCAGGAGC | 461 |
| hDMD-E50g26-top | CACCGTCCTGGACTGACCACTATTG | 462 |
| hDMD-E50g26-bottom | aaacCAATAGTGGTCAGTCCAGGA | 463 |
| hDMD-E50g27-top | CACCGTGACCACTATTGGAGCCTGT | 464 |
| hDMD-E50g27-bottom | aaacACAGGCTCCAATAGTGGTCA | 465 |
| hDMD-E50g28-top | CACCGATGGGATCCAGTATACTTAC | 466 |
| hDMD-E50g28-bottom | aaacGTAAGTATACTGGATCCCAT | 467 |
| hDMD-E50g29-top | CACCGAATGGGATCCAGTATACTTA | 468 |
| hDMD-E50g29-bottom | aaacTAAGTATACTGGATCCCATT | 469 |
| hDMD-E50g30-top | CACCGATTGGAGCCTGTAAGTATAC | 470 |
| hDMD-E50g30-bottom | aaacATTGGAGCCTGTAAGTATAC | 471 |
| hDMD-E51g4-top | CACCGTCATCTCGTTGATATCCTCA | 472 |
| hDMD-E51g4-bottom | aaacTGAGGATATCAACGAGATGA | 473 |
| hDMD-E51g5-top | CACCGCGAGATGATCATCAAGCAGA | 474 |
| hDMD-E51g5-bottom | aaacTCTGCTTGATGATCATCTCG | 475 |
| hDMD-E51g6-top | CACCGTGACCTTGAGGATATCAAC | 476 |
| hDMD-E51g6-bottom | aaacGTTGATATCCTCAAGGTCAC | 477 |
| hDMD-E51g7-top | CACCGTCAACGAGATGATCATCAAG | 478 |
| hDMD-E51g7-bottom | aaacCTTGATGATCATCTCGTTGA | 479 |
| hDMD-E51g8-top | CACCGACGAGATGATCATCAAGCAG | 480 |
| hDMD-E51g8-bottom | aaacCTGCTTGATGATCATCTCGT | 481 |
| hDMD-E53g1-top | CACCGATTTATTTTTCCTTTTATTC | 482 |
| hDMD-E53g1-bottom | aaacGAATAAAAGGAAAAATAAAT | 483 |
| hDMD-E53g2-top | CACCGTTTCCTTTTATTCTAGTTGA | 484 |
| hDMD-E53g2-bottom | aaacTCAACTAGAATAAAAGGAAA | 485 |
| hDMD-E53g3-top | CACCGTGATTCTGAATTCTTTCAAC | 486 |
| hDMD-E53g3-bottom | aaacGTTGAAAGAATTCAGAATCA | 487 |
| hDMD-E53g4-top | CACCGAAAGAAAATCACAGAAACCA | 488 |
| hDMD-E53g4-bottom | aaacTGGTTTCTGTGATTTTCTTT | 489 |
| hDMD-E53g5-top | CACCGAAAATCACAGAAACCAAGGT | 490 |
| hDMD-E53g5-bottom | aaacACCTTGGTTTCTGTGATTTT | 491 |
| hDMD-E53g6-top | CACCGGTATCTTTGATACTAACCT | 492 |
| hDMD-E53g6-bottom | aaacAGGTTAGTATCAAAGATACC | 493 |
| hDMD-E53g7-bottom | aaacACTGATTCTGAATTCTTTCAC | 47 |
| hDMD-E53g7-top | CACCGTGAAAGAATTCAGAATCAGT | 48 |
| hDMD-E53g8-bottom | aaacTCAGAACCGGAGGCAACAGTC | 49 |
| hDMD-E53g8-top | CACCGACTGTTGCCTCCGGTTCTGA | 50 |
| hDMD-E53g9-top | CACCGTACAAGAACACCTTCAGAAC | 51 |
| hDMD-E53g9-bottom | aaacGTTCTGAAGGTGTTCTTGTAC | 52 |
| hDMD-E53g10-bottom | aaacCCGGTTCTGAAGGTGTTCTTC | 53 |
| hDMD-E53g10-top | CACCGAAGAACACCTTCAGAACCGG | 54 |
| hDMD-E53g11-bottom | aaacGAGGCAACAGTTGAATGAAAC | 55 |
| hDMD-E53g11-top | CACCGTTTCATTCAACTGTTGCCTC | 56 |
| hDMD-E53g12-top | CACCGTGTTAAAGGATTCAACACAA | 57 |
| hDMD-E53g12-bottom | aaacTTGTGTTGAATCCTTTAACAC | 58 |
| hDMD-E53g13-bottom | aaacGCCATTGTGTTGAATCCTTTC | 59 |
| hDMD-E53g13-top | CACCGAAAGGATTCAACACAATGGC | 60 |
| hDMD-E53g14-top | CACCGAATTCAGAATCAGTGGGATG | 494 |
| hDMD-E53g14-bottom | aaacCATCCCACTGATTCTGAATT | 495 |
| hDMD-E53g15-bottom | aaacCTGATTCTGAATTCTTTCAAC | 496 |
| hDMD-E53g15-top | CACCGTTGAAAGAATTCAGAATCAG | 497 |
| hDMD-E53g16-top | CACCGACAGTTGAATGAAATGTTAA | 498 |
| hDMD-E53g16-bottom | aaacTTAACATTTCATTCAACTGTC | 499 |
| hDMD-E53g17-top | CACCGACCTTCAGAACCGGAGGCAA | 500 |
| hDMD-E53g17-bottom | aaacTTGCCTCCGGTTCTGAAGGTC | 501 |
| hDMD-E53g18-top | CACCGAATTCTTTCAACTAGAATAA | 502 |
| hDMD-E53g18-bottom | aaacTTATTCTAGTTGAAAGAATTC | 503 |
| hDMD-E53g19-top | CACCGTTATTCTAGTTGAAAGAATT | 504 |
| hDMD-E53g19-bottom | aaacAATTCTTTCAACTAGAATAAC | 505 |
| hDMD-E53g20-top | CACCGTAGTTGAAAGAATTCAGAAT | 506 |
| hDMD-E53g20-bottom | aaacATTCTGAATTCTTTCAACTAC | 507 |
| hDMD-E53g21-top | CACCGATGAAGTACAAGAACACCTT | 508 |
| hDMD-E53g21-bottom | aaacAAGGTGTTCTTGTACTTCATC | 509 |
| hDMD-E53g22-top | CACCGAACTGTTGCCTCCGGTTCTG | 510 |
| hDMD-E53g22-bottom | aaacCAGAACCGGAGGCAACAGTTC | 511 |
| hDMD-E53g23-top | CACCGCAAGAACACCTTCAGAACCG | 512 |
| hDMD-E53g23-bottom | aaacCGGTTCTGAAGGTGTTCTTGC | 513 |
| hDMD-E53g24-top | CACCGCAAGAACACCTTCAGAACCG | 514 |
| hDMD-E53g24-bottom | aaacCGGTTCTGAAGGTGTTCTTGC | 515 |

TABLE 9
Sequence of primers for generating sgRNA targeting mouse
Dmd exon 44, exon 46, and exon 53

| ID | Sequence (5′-3′) | SEQ ID NO. |
|---|---|---|
| mDmd-E43g1-top | CACCGATTTGCAACAAATCTCAGGT | 516 |
| mDmd-E43g1-bottom | aaacACCTGAGATTTGTTGCAAAT | 517 |
| mDmd-E43g2-top | CACCGAGAATGTACAAGGAACGACA | 518 |
| mDmd-E43g2-bottom | aaacTGTCGTTCCTTGTACATTCT | 519 |
| mDmd-E43g3-top | CACCGAATGTACAAGGAACGACAA | 520 |
| mDmd-E43g3-bottom | aaacTTGTCGTTCCTTGTACATTC | 521 |
| mDmd-E43g4-bottom | aaacACCCTTGTCGTTCCTTGTAC | 522 |
| mDmd-E43g4-top | CACCGTACAAGGAACGACAAGGGT | 523 |
| mDmd-E44g1-bottom | aaacATAATTTGAAAACATGGATGC | 15 |
| mDmd-E44g1-top | CACCGCATCCATGTTTTCAAATTAT | 16 |
| mDmd-E44g2-bottom | aaacTTTTTCAACTGATCTGTCGAC | 17 |
| mDmd-E44g2-top | CACCGTCGACAGATCAGTTGAAAAA | 18 |
| mDmd-E44g3-bottom | aaacGTTTTCAGGATTTTGTGTCTC | 19 |
| mDmd-E44g3-top | CACCGAGACACAAAATCCTGAAAAC | 20 |
| mDmd-E44g4-bottom | aaacGAAAACTGGGAACATGCTAAC | 21 |
| mDmd-E44g4-top | CACCGTTAGCATGTTCCCAGTTTTC | 22 |
| mDmd-E44g5-bottom | aaacAGTTTTCAGGATTTTGTGTC | 23 |
| mDmd-E44g5-top | CACCGACACAAAATCCTGAAAACT | 24 |
| mDmd-E44g6-bottom | aaacTTTGTATTTAGCATGTTCCC | 25 |
| mDmd-E44g6-top | CACCGGGAACATGCTAAATACAAA | 26 |
| mDmd-E44g7-bottom | aaacTAAGATACCATTTGTATTTAC | 27 |
| mDmd-E44g7-top | CACCGTAAATACAAATGGTATCTTA | 28 |
| mDmd-E44g8-bottom | aaacTAATTTGAAAACATGGATGAC | 524 |
| mDmd-E44g8-top | CACCGTCATCCATGTTTTCAAATTA | 525 |
| mDmd-E44g9-bottom | aaacTCGAATCGCCTATAATTTGAC | 526 |
| mDmd-E44g9-top | CACCGTCAAATTATAGGCGATTCGA | 527 |
| mDmd-E44g10-bottom | aaacAATGAAGTTGAACAGTTTTTC | 528 |
| mDmd-E44g10-top | CACCGAAAAACTGTTCAACTTCATT | 529 |
| mDmd-E44g11-bottom | aaacTTCAACTTCATTCAGCCATTC | 530 |
| mDmd-E44g11-top | CACCGAATGGCTGAATGAAGTTGAA | 531 |
| mDmd-E44g12-top | CACCGAAGTTGAACAGTTTTTCAAA | 532 |
| mDmd-E44g12-bottom | aaacTTTGAAAAACTGTTCAACTTC | 533 |
| mDmd-E44g13-bottom | aaacAAAACTGGGAACATGCTAAAC | 534 |
| mDmd-E44g13-top | CACCGTTTAGCATGTTCCCAGTTTT | 535 |
| mDmd-E44g14-bottom | aaacAATACAAATGGTATCTTAAGC | 536 |
| mDmd-E44g14-top | CACCGCTTAAGATACCATTTGTATT | 537 |
| mDmd-E44g15-bottom | aaacAAGATACCATTTGTATTTAGC | 538 |
| mDmd-E44g15-top | CACCGCTAAATACAAATGGTATCTT | 539 |
| mDmd-E44g16-bottom | aaacACCTTAAGATACCATTTGTAC | 540 |
| mDmd-E44g16-top | CACCGTACAAATGGTATCTTAAGGT | 541 |
| mDmd-E44g17-bottom | aaacAAGGTAAGACTTTGAGATTTC | 542 |
| mDmd-E44g17-top | CACCGAAATCTCAAAGTCTTACCTT | 543 |
| mDmd-E44g18-top | CACCGTTATAGGCGATTCGACAGAT | 544 |
| mDmd-E44g18-bottom | aaacATCTGTCGAATCGCCTATAA | 545 |
| mDmd-E44g19-top | CACCGcatggatgaaataaggtaag | 546 |
| mDmd-E44g19-bottom | aaacCTTACCTTATTTCATCCATG | 547 |
| mDmd-E44g20-top | CACCGctgaaaaaatgaagccagca | 548 |
| mDmd-E44g20-bottom | aaacTGCTGGCTTCATTTTTTCAG | 549 |
| mDmd-E44g21-top | CACCGATTTAATCAATGGCTGAATG | 550 |
| mDmd-E44g21-bottom | aaacCATTCAGCCATTGATTAAAT | 551 |
| mDmd-E46g1-top | CACCGAATTTTGTTATTCTTAATAC | 37 |
| mDmd-E46g1-bottom | aaacGTATTAAGAATAACAAAATTC | 38 |
| mDmd-E46g2-bottom | aaacGCCACAAAACAAATTCATTTC | 39 |
| mDmd-E46g2-top | CACCGAAATGAATTTGTTTTGTGGC | 40 |
| mDmd-E46g3-bottom | aaacAGTGGAGTAATAGCAATGTTC | 41 |
| mDmd-E46g3-top | CACCGAACATTGCTATTACTCCACT | 42 |
| mDmd-E46g4-top | CACCGAGCTGCTGCTCATCTCCAAG | 43 |
| mDmd-E46g4-bottom | aaacCTTGGAGATGAGCAGCAGCTC | 44 |
| mDmd-E46g5-top | CACCGAGAACAACTTGAACAAGTCA | 45 |
| mDmd-E46g5-bottom | aaacTGACTTGTTCAAGTTGTTCTC | 46 |
| mDmd-E46g6-bottom | aaacTATTAAGAATAACAAAATTC | 552 |
| mDmd-E46g6-top | CACCGAATTTTGTTATTCTTAATA | 553 |
| mDmd-E46g7-top | CACCGTTGTTCTTCAATCCTGTATT | 554 |
| mDmd-E46g7-bottom | aaacAATACAGGATTGAAGAACAAC | 555 |
| mDmd-E46g8-bottom | aaacCAATCCTGTATTAAGAATAAC | 556 |
| mDmd-E46g8-top | CACCGTTATTCTTAATACAGGATTG | 557 |
| mDmd-E46g9-bottom | aaacTTGTTCTTCAATCCTGTATTC | 558 |
| mDmd-E46g9-top | CACCGAATACAGGATTGAAGAACAA | 559 |
| mDmd-E46g10-bottom | aaacCTCATCTCCAAGTGGAGTAAC | 560 |
| mDmd-E46g10-top | CACCGTTACTCCACTTGGAGATGAG | 561 |
| mDmd-E46g11-bottom | aaacGTTCAAGTTGTTCTTTTAGC | 562 |
| mDmd-E46g11-top | CACCGCTAAAAGAACAACTTGAAC | 563 |
| mDmd-E46g12-top | CACCGAAATTACCTTGACTTGTTC | 564 |
| mDmd-E46g12-bottom | aaacGAACAAGTCAAGGTAATTTC | 565 |
| mDmd-E46g13-top | CACCGAAGAACAACTTGAACAAGTC | 566 |
| mDmd-E46g13-bottom | aaacGACTTGTTCAAGTTGTTCTTC | 567 |
| mDmd-E46g14-top | CACCGACTTGTTCAAGTTGTTCTTT | 568 |
| mDmd-E46g14-bottom | aaacAAAGAACAACTTGAACAAGT | 569 |
| mDmd-E46g15-top | CACCGacacctctcagggatttagg | 570 |
| mDmd-E46g15-bottom | aaacCCTAAATCCCTGAGAGGTGT | 571 |
| mDmd-E46g16-top | CACCGttcccttattaaaatcctca | 572 |
| mDmd-E46g16-bottom | aaacTGAGGATTTTAATAAGGGAA | 573 |
| mDmd-E46g17-top | CACCGctttatacaaataggccctg | 574 |
| mDmd-E46g17-bottom | aaacCAGGGCCTATTTGTATAAAG | 575 |
| mDmd-E51g4-top | CACCGTGAAATGATCATCAAACAGA | 576 |
| mDmd-E51g4-bottom | aaacTCTGTTTGATGATCATTTCA | 577 |
| mDmd-E51g5-top | CACCGTCAATGAAATGATCATCAAA | 578 |
| mDmd-E51g5-bottom | aaacTTTGATGATCATTTCATTGA | 579 |
| mDmd-E51g6-top | CACCGATGAAATGATCATCAAACAG | 580 |
| mDmd-E51g6-bottom | aaacCTGTTTGATGATCATTTCAT | 581 |
| mDmd-E51g7-top | CACCGTGATCATCAAACAGAAGGTA | 582 |
| mDmd-E51g7-bottom | aaacTACCTTCTGTTTGATGATCA | 583 |
| mDmd-E53g1-top | CACCGTGAAAGAATTCAGATTCAGT | 61 |
| mDmd-E53g1-bottom | aaacACTGAATCTGAATTCTTTCAC | 62 |
| mDmd-E53g2-bottom | aaacCATCCCACTGAATCTGAATTC | 63 |
| mDmd-E53g2-top | CACCGAATTCAGATTCAGTGGGATG | 64 |
| mDmd-E53g3-bottom | aaacGTTCTGCAGCTGTTCTTGAAC | 65 |
| mDmd-E53g3-top | CACCGTTCAAGAACAGCTGCAGAAC | 66 |
| mDmd-E53g4-bottom | aaacTTAACATTTCATTCAACTGTC | 67 |
| mDmd-E53g4-top | CACCGACAGTTGAATGAAATGTTAA | 68 |
| mDmd-E53g5-bottom | aaacTTGTGTTGAATCCTTTAACAC | 69 |
| mDmd-E53g5-top | CACCGTGTTAAAGGATTCAACACAA | 70 |
| mDmd-E53g6-bottom | aaacGCCATTGTGTTGAATCCTTTC | 71 |
| mDmd-E53g6-top | CACCGAAAGGATTCAACACAATGGC | 72 |
| mDmd-E53g7-top | CACCGAAAGAAGATCACAGAAACCA | 584 |
| mDmd-E53g7-bottom | aaacTGGTTTCTGTGATCTTCTTT | 585 |
| mDmd-E53g8-top | CACCGTTGAAAGAATTCAGATTCAG | 586 |
| mDmd-E53g8-bottom | aaacCTGAATCTGAATTCTTTCAA | 587 |
| mDmd-E53g9-top | CACCGAGTGGGATGAGGTTCAAGAA | 588 |
| mDmd-E53g9-bottom | aaacTTCTTGAACCTCATCCCACTC | 589 |
| mDmd-E53g10-top | CACCGAGCTGCAGAACAGGAGACAA | 590 |
| mDmd-E53g10-bottom | aaacTTGTCTCCTGTTCTGCAGCTC | 591 |
| mDmd-E53g11-top | CACCGTGAATCTGAATTCTTTCAAC | 592 |
| mDmd-E53g11-bottom | aaacGTTGAAAGAATTCAGATTCAC | 593 |
| mDmd-E53g12-top | CACCGCTTTCAACTGGAATAAAAAT | 594 |
| mDmd-E53g12-bottom | aaacATTTTTATTCCAGTTGAAAGC | 595 |
| mDmd-E53g13-top | CACCGCTTATTTTTATTCCAGTTGA | 596 |
| mDmd-E53g13-bottom | aaacTCAACTGGAATAAAAATAAGC | 597 |
| mDmd-E53g14-top | CACCGTTATTCCAGTTGAAAGAATT | 598 |
| mDmd-E53g14-bottom | aaacAATTCTTTCAACTGGAATAAC | 599 |
| mDmd-E53g15-top | CACCGCAGTTGAAAGAATTCAGATT | 600 |
| mDmd-E53g15-bottom | aaacAATCTGAATTCTTTCAACTGC | 601 |
| mDmd-E53g16-top | CACCGAATTCAGATTCAGTGGGAT | 602 |
| mDmd-E53g16-bottom | aaacATCCCACTGAATCTGAATTC | 603 |
| mDmd-E53g17-top | CACCGATTCAGTGGGATGAGGTTC | 604 |
| mDmd-E53g17-bottom | aaacGAACCTCATCCCACTGAATC | 605 |
| mDmd-E53g18-top | CACCGATGAGGTTCAAGAACAGCTG | 606 |
| mDmd-E53g18-bottom | aaacCAGCTGTTCTTGAACCTCATC | 607 |
| mDmd-E53g19-top | CACCGTTCAAGAACAGCTGCAGAA | 608 |
| mDmd-E53g19-bottom | aaacTTCTGCAGCTGTTCTTGAAC | 609 |
| mDmd-E53g20-top | CACCGAACTGTTGTCTCCTGTTCTG | 610 |
| mDmd-E53g20-bottom | aaacCAGAACAGGAGACAACAGTTC | 611 |
| mDmd-E53g21-top | CACCGCAAGAACAGCTGCAGAACAG | 612 |
| mDmd-E53g21-bottom | aaacCTGTTCTGCAGCTGTTCTTGC | 613 |
| mDmd-E53g22-top | CACCGAAGATCACAGAAACCAAGGT | 614 |
| mDmd-E53g22-bottom | aaacACCTTGGTTTCTGTGATCTTC | 615 |
| mDmd-E53g23-top | CACCGCAGAAACCAAGGTTAGTGTC | 616 |
| mDmd-E53g23-bottom | aaacGACACTAACCTTGGTTTCTGC | 2261 |

TABLE 10
Sequence of primers for generating sgRNA targeting Dmd exon 43,
exon 45, and exon 52 to generate the mouse models

| | Mouse | | SEQ |
|---|---|---|---|
| ID | Model | Sequence (5′-3′) | ID NO. |
| mDmd-Ex43-N1-Top | Δ43 DMD | caccgaagtgagttccaggacagcc | 73 |
| mDmd-Ex43-N1-Bottom | Δ43 DMD | aaacGGCTGTCCTGGAACTCACTTc | 74 |
| mDmd-Ex43-N2-Top | Δ43 DMD | caccgTTATTAGTACTAACTCAGAA | 75 |
| mDmd-Ex43-N2-Bottom | Δ43 DMD | aaacTTCTGAGTTAGTACTAATAAc | 76 |
| mDmd-Ex43-N3-Top | Δ43 DMD | caccgtagtactaataaaagtagtc | 77 |
| mDmd-Ex43-N3-Bottom | Δ43 DMD | aaacGACTACTTTTATTAGTACTAc | 78 |
| mDmd-Ex43-C1-Top | Δ43 DMD | caccgATGTTGAAATACACTGCTCT | 79 |
| mDmd-Ex43-C1-Bottom | Δ43 DMD | aaacAGAGCAGTGTATTTCAACATc | 80 |
| mDmd-Ex43-C2-Top | Δ43 DMD | caccgGTAAATATCAACTTCTAAAT | 81 |
| mDmd-Ex43-C2-Bottom | Δ43 DMD | aaacATTTAGAAGTTGATATTTACc | 82 |
| mDmd-Ex43-C3-Top | Δ43 DMD | caccggtatttccctatttttaatg | 83 |
| mDmd-Ex43-C3-Bottom | Δ43 DMD | aaacCATTAAAAATAGGGAAATACc | 84 |
| mDmd-Exon45-5-G1-top | Δ45 DMD | CACCGaactaatatatccaaatact | 85 |
| mDmd-Exon45-5-G1-bot | Δ45 DMD | AAACAGTATTTGGATATATTAGTT C | 86 |
| mDmd-Exon45-5-G2-top | Δ45 DMD | CACCGttaaacatagaacatccttg | 87 |
| mDmd-Exon45-5-G2-bot | Δ45 DMD | AAACCAAGGATGTTCTATGTTTAA C | 88 |
| mDmd-Exon45-5-G3-top | Δ45 DMD | CACCGaatcgaatttgctcttgaga | 89 |
| mDmd-Exon45-5-G3-bot | Δ45 DMD | AAACTCTCAAGAGCAAATTCGATT C | 90 |
| mDmd-Exon45-3-G4-top | Δ45 DMD | CACCGagtttgtgctaaaaatcatg | 91 |
| mDmd-Exon45-3-G4-bot | Δ45 DMD | AAACCATGATTTTTAGCACAAACT C | 92 |
| mDmd-Exon45-3-G5-top | Δ45 DMD | CACCGgctttacccagctgaatcac | 93 |
| mDmd-Exon45-3-G5-bot | Δ45 DMD | AAACGTGATTCAGCTGGGTAAAGC C | 94 |
| mDmd-Exon45-3-G6-top | Δ45 DMD | CACCGaattttcacagcattgctta | 95 |
| mDmd-Exon45-3-G6-bot | Δ45 DMD | AAACTAAGCAATGCTGTGAAAATT C | 96 |
| mDmd-Ex52-N1-Top | Δ52 DMD | CACCGATATATCTTAAATGATGTAT | 97 |
| mDmd-Ex52-N1-bottom | Δ52 DMD | AAACATACATCATTTAAGATATATC | 98 |
| mDmd-Ex52-N2-Top | Δ52 DMD | CACCGAATAATAATGCTGTTTGATG | 99 |
| mDmd-Ex52-N2-bottom | Δ52 DMD | aaacCATCAAACAGCATTATTATTc | 100 |
| mDmd-Ex52-N3-Top | Δ52 DMD | CACCGGATAGTTAGAAATGACTCCA | 101 |
| mDmd-Ex52-N3-bottom | Δ52 DMD | AAACTGGAGTCATTTCTAACTATCC | 102 |
| mDmd-Ex52-C1-Top | Δ52 DMD | CACCGtctttaatgtctgtctacta | 103 |
| mDmd-Ex52-C1-Bottom | Δ52 DMD | aaacTAGTAGACAGACATTAAAGAc | 104 |
| mDmd-Ex52-C2-Top | Δ52 DMD | caccgtgcaatttcatagtatattt | 105 |
| mDmd-Ex52-C2-Bottom | Δ52 DMD | aaacAAATATACTATGAAATTGCAc | 106 |
| mDmd-Ex52-C3-Top | Δ52 DMD | caccgccaagttaatcaaattgttc | 107 |
| mDmd-Ex52-C3-Bottom | Δ52 DMD | aaacGAACAATTTGATTAACTTGGc | 108 |

TABLE 11
Sequence of primers
for in vitro transcription of sgRNA

| | Mouse | | SEQ ID |
|---|---|---|---|
| ID | Model | Sequence (5′-3′) | NO. |
| IVTT7-mDmd- | Δ43 | gaattgTAATACGACTCACTATA | 109 |
| E43-N1-F | DMD | GGGaagtgagttccaggacagcc | |
| IVTT7-mDmd- | Δ43 | gaattgTAATACGACTCACTATA | 110 |
| E43-N2-F | DMD | GGGTTATTAGTACTAACTCAGAA | |
| IVTT7-mDmd- | Δ43 | gaattgTAATACGACTCACTATA | 111 |
| E43-N3-F | DMD | GGGtagtactaataaaagtagtc | |
| IVTT7-mDmd- | Δ43 | gaattgTAATACGACTCACTATA | 112 |
| E43-C1-F | DMD | GGGATGTTGAAATACACTGCTCT | |
| IVTT7-mDmd- | Δ43 | gaattgTAATACGACTCACTATA | 113 |
| E43-C2-F | DMD | GGGGTAAATATCAACTTCTAAAT | |
| IVTT7-mDmd- | Δ43 | gaattgTAATACGACTCACTATA | 114 |
| E43-C3-F | DMD | GGGgtatttccctatttttaatg | |
| IVTT7-mDmd- | Δ52 | gaattgTAATACGACTCACTATA | 115 |
| E52-N1-F | DMD | GGGatatatcttaaatgatgtat | |
| IVTT7-mDmd- | Δ52 | gaattgTAATACGACTCACTATA | 116 |
| E52-N2-F | DMD | GGGaataataatgctgtttgatg | |
| IVTT7-mDmd- | Δ52 | gaattgTAATACGACTCACTATA | 117 |
| E52-N3-F | DMD | GGGGATAGTTAGAAATGACTCCA | |
| IVTT7-mDmd- | Δ52 | gaattgTAATACGACTCACTATA | 118 |
| E52-C1-F | DMD | GGGtctttaatgtctgtctacta | |
| IVTT7-mDmd- | Δ52 | gaattgTAATACGACTCACTATA | 119 |
| E52-C2-F | DMD | GGGtgcaatttcatagtatattt | |
| IVTT7-mDmd- | Δ52 | gaattgTAATACGACTCACTATA | 120 |
| E52-C3-F | DMD | GGGccaagttaatcaaattgttc | |
| T7-Rv | All | AAAAGCACCGACTCGGTGCCAC | 121 |

TABLE 12
Sequence of primers for genotyping

| | Mouse | | SEQ ID |
|---|---|---|---|
| ID | Model | Sequence (5′-3′) | NO. |
| mDmd-Ex43- | Δ43 DMD | gcacacctttaatcccagca | 122 |
| geno-F1 | | | |
| mDmd-Ex43- | Δ43 DMD | tctgcagccgaataccttca | 123 |
| geno-R1 | | | |
| mDmd-Ex45- | Δ45 DMD | tctcattctgtgcattcttggt | 124 |
| geno-F1 | | | |
| mDmd-Ex45- | Δ45 DMD | gctttccaattaccatagcatgc | 125 |
| geno-R2 | | | |
| mDmd-Ex52- | Δ52 DMD | agggaatctgctgtccttga | 126 |
| geno-F1 | | | |
| mDmd-Ex52- | Δ52 DMD | tggaggttagatttcacaactgt | 127 |
| geno-R1 | | | |

### VII. EXAMPLES

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1

### Materials and Methods

Study Approval. All experimental procedures involving animals in this study were reviewed and approved by the University of Texas Southwestern Medical Center's Institutional Animal Care and Use Committee.

Plasmids. The pSpCas9(BB)-2A-GFP (PX458) plasmid containing the human codon optimized SpCas9 gene with 2A-EGFP and the backbone of sgRNA was purchased from Addgene (Plasmid #48138). Cloning of sgRNA was done using Bbs I sites. The AAV TRISPR-CK8-GFP plasmid containing three sgRNAs driven by U6, H1 or 7SK promoter and GFP driven by CK8 promoter.

Human iPSCs maintenance and nucleofection. Human iPSCs were cultured in mTeSR™ 1 media (STEMCELL Technologies) and passaged approximately every 4 days (1:14 split ratio). One hour before nucleofection, iPSCs were treated with 10 μM ROCK inhibitor (Y-27632) and dissociated into single cells using Accutase (Innovative Cell Technologies, Inc.). 1×10⁶ iPSCs were mixed with 5 μg of pX458-gRNA-2A-GFP plasmid and nucleofected using the P3 Primary Cell 4D-Nucleofector X kit (Lonza) according to manufacturer's protocol. After nucleofection, iPSCs were cultured in mTeSR™ 1 media supplemented with 10 μM ROCK inhibitor, penicillin-streptomycin (1:100) (ThermoFisher Scientific) and 100 μg/ml Primosin (InvivoGen). Three days post-nucleofection, GFP(+) and (−) cells were sorted by FACS and subjected to T7E1 assay. Single clones derived from GFP(+) iPSCs were picked and sequenced.

Genomic DNA isolation. Genomic DNA of mouse 10T1/2 fibroblasts, mouse N2a cells, human 293 and human iPSCs was isolated using DirectPCR (cell) lysis reagent (VIAGEN) according to manufacturer's protocol.

PCR amplification of genomic DNA. Genomic DNA was PCR-amplified using GoTaq DNA polymerase (Promega) with primers. PCR products were gel purified and subcloned into pCRII-TOPO vector (Invitrogen) according to the manufacturer's protocol. Individual clones were picked and the DNA was sequenced.

T7E1 analysis of PCR products. Mismatched duplex DNA was obtained by denaturing/renaturing of 25 μl of the genomic PCR product using the following conditions: 95° C. for 5 mins, 95° C. to 85° C. (−2.0° C./seconds), 85° C. to 25° C. (−0.1° C./seconds), hold at 4° C. Then 25 μl of the mismatched duplex DNA was incubated with 2.7 μl of 10× NEB buffer 2 and 0.3 μl of T7E1 (New England BioLabs) at 37° C. for 90 minutes. The T7E1 digested PCR product was analyzed by 2% agarose gel electrophoresis.

Human cardiomyocyte differentiation. Human iPSCs were cultured in mTeSR™ 1 media for 3 days until they reached 90% confluence. To differentiate the iPSCs to cardiomyocytes, the iPSCs were cultured in CDM3-C media for 2 days, followed by CDM3-59 media for 2 days, followed by CDM3 media for 6 days, followed by selective media for 10 days and lastly by basal media for 2 days. Then, the cardiomyocytes were dissociated using TrypLE media and re-plated at 2×10⁶ per well in a 6-well dish.

Dystrophin Western blot analysis. After 30 days post-differentiation, 2×10⁶ cardiomyocytes were harvested and lysed with lysis buffer (10% SDS, 62.5 mM Tris pH=6.8, 1 mM EDTA, and protease inhibitor). Cell lysates were passed through a 22 G syringe and then a 27 G syringe, 10 times each. Protein concentration was determined by BCA assay and 50 ug of total protein was loaded onto an acrylamide gel. After running at 100V (20 mA) for 5 hours and followed by 1 hour 20 min transfer to PVDF membrane at 35V (200 mA) at 4° C. The blot was incubated with mouse anti-dystrophin antibody (MANDYS8, Sigma-Aldrich, D8168) at 4° C. overnight and with goat anti-mouse HRP antibody (Bio-Rad Laboratories) at RT for 1 hour. The blot was developed using Western Blotting Luminol Reagent (Santa Cruz, sc-2048). The loading control was determined by blotting with mouse anti-vinculin antibody (Sigma-Aldrich, V9131).

CRISPR/Cas9-mediated exon deletion in mice. Two single-guide RNA (sgRNA) specific intronic regions surrounding exon 43, exon 45, or exon 52 sequence of the mouse Dmd locus were cloned into vector pX458 using the primers from Table 10. For the in vitro transcription of sgRNA, T7 promoter sequence was added to the sgRNA template by PCR using the primers from Table 11. The gel purified PCR products were used as template for in vitro transcription using the MEGAshortscript T7 Kit (Life Technologies). sgRNA were purified by MEGAclear kit (Life Technologies) and eluted with nuclease-free water (Ambion). The concentration of guide RNA was measured by a NanoDrop instrument (Thermo Scientific).

Genotyping of 443, 445, and 452 DMD Mice. 443, 445, and 452 DMD mice were genotyped using primers encompassing the targeted region from Table 12. Tail biopsies were digested in 100 μL of 25 mM NaOH, 0.2 mM EDTA (pH 12) for 20 min at 95° C. Tails were briefly centrifuged followed by addition of 100 μL of 40 mM Tris.HCl (pH 5) and mixed to homogenize. Two microliters of this reaction was used for subsequent PCR reactions with the primers below, followed by gel electrophoresis.

Histological analysis of muscles. Skeletal muscles from WT, Δ43 DMD, Δ45 DMD, and Δ52 DMD mice were individually dissected and cryoembedded in a 1:2 volume mixture of Gum Tragacanth powder (Sigma-Aldrich) to Tissue Freezing Medium (TFM) (Triangle Bioscience). All embeds were snap frozen in isopentane heat extractant supercooled to −155° C. Resulting blocks were stored overnight at −80° C. prior to sectioning. Eight-micron transverse sections of skeletal muscle, and frontal sections of heart were prepared on a Leica CM3050 cryostat and air-dried prior to same day staining. H&E staining was performed according to established staining protocols and dystrophin immunohistochemistry was performed using MANDYS8 monoclonal antibody (Sigma-Aldrich) with modifications to manufacturer's instructions. In brief, cryostat sections were thawed and rehydrated/delipidated in 1% triton/phosphate-buffered-saline, pH 7.4 (PBS). Following delipidation, sections were washed free of Triton, incubated with mouse IgG blocking reagent (M.O.M. Kit, Vector Laboratories), washed, and sequentially equilibrated with MOM protein concentrate/PBS, and MANDYS8 diluted 1:1800 in MOM protein concentrate/PBS. Following overnight primary antibody incubation at 4° C., sections were washed, incubated with MOM biotinylated anti-mouse IgG, washed, and detection completed with incubation of Vector fluorescein-avidin DCS. Nuclei were counterstained with propidium iodide (Molecular Probes) prior to cover slipping with Vectashield.

### Example 2

### Results

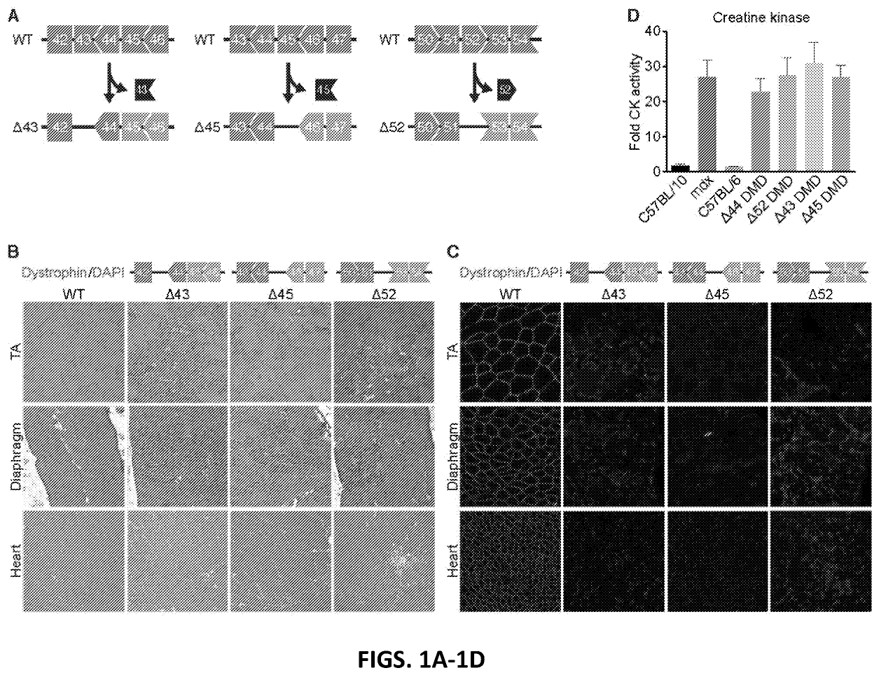

Δ43, Δ45, and Δ52 DMD mouse models recapitulate muscle dystrophy phenotype. To investigate CRISPR/Cas9-mediated exon skipping and reframing in vivo, three mimics of the human “hot spot” regions were generated in three mouse models by deleting the exon 43, exon 45, and exon 52, respectively, using CRISPR/Cas9 system directed by 2 single guide RNAs (sgRNA) (FIG. 1A and Table 9). The inventors designed and validated sgRNAs targeting 5′ end and 3′ end of Dmd exon 43, exon 45, and exon 52. C57BL/6 zygotes were co-injected with in vitro transcribed Cas9 mRNA and in vitro transcribed sgRNAs, and then re-implanted into pseudo-pregnant females.

The deletion of Dmd exon 43, exon 45, and exon 52 was confirmed by DNA genotyping. 1-month old mice lacking exon 43, exon 45, or exon 52 showed pronounced dystrophic muscle changes. (FIG. 1B). The deletion of these exons placed the dystrophin gene out of frame leading to the absence of dystrophin protein in skeletal muscle and heart (FIG. 1C). Serum analysis of the Δ43, Δ45, and Δ52 DMD mice shows a significant increase of creatine kinase (CK) level, which is a sign of muscle damage (FIG. 1D). Taken together, dystrophin protein expression, muscle histology, and serum creatine kinase level validated dystrophic phenotype of the Δ43, Δ45, and Δ52 DMD mouse models.

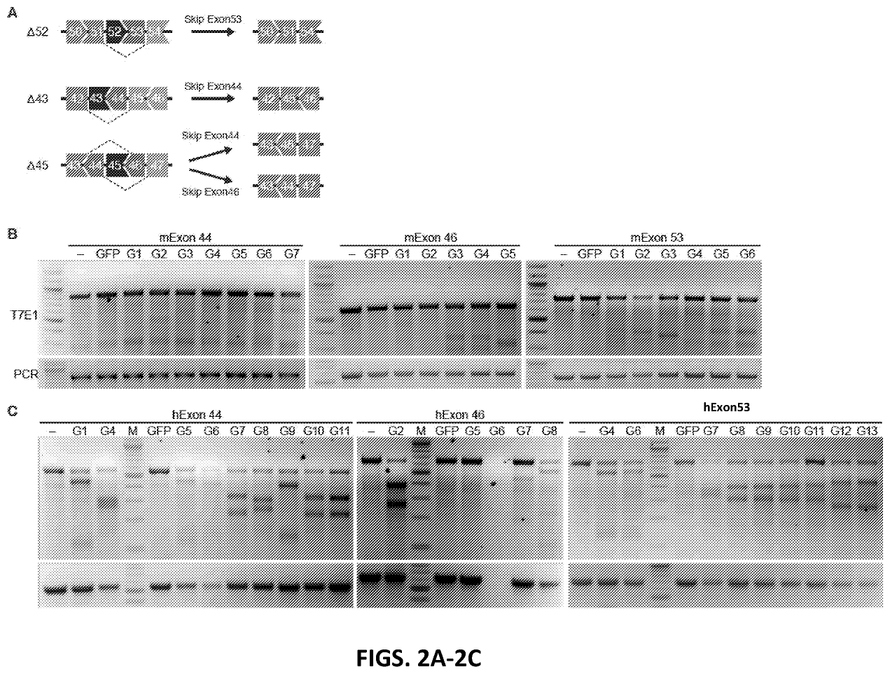

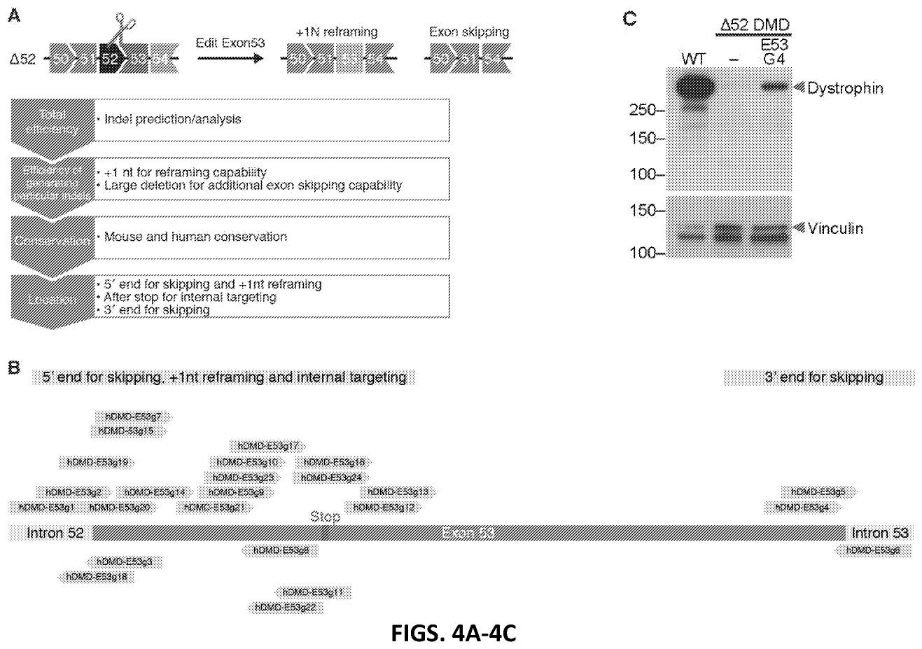

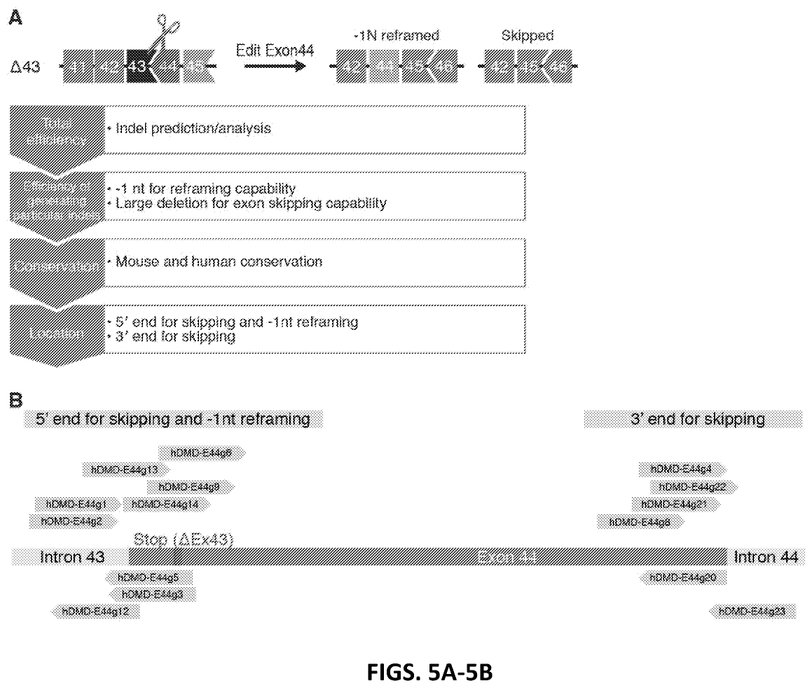

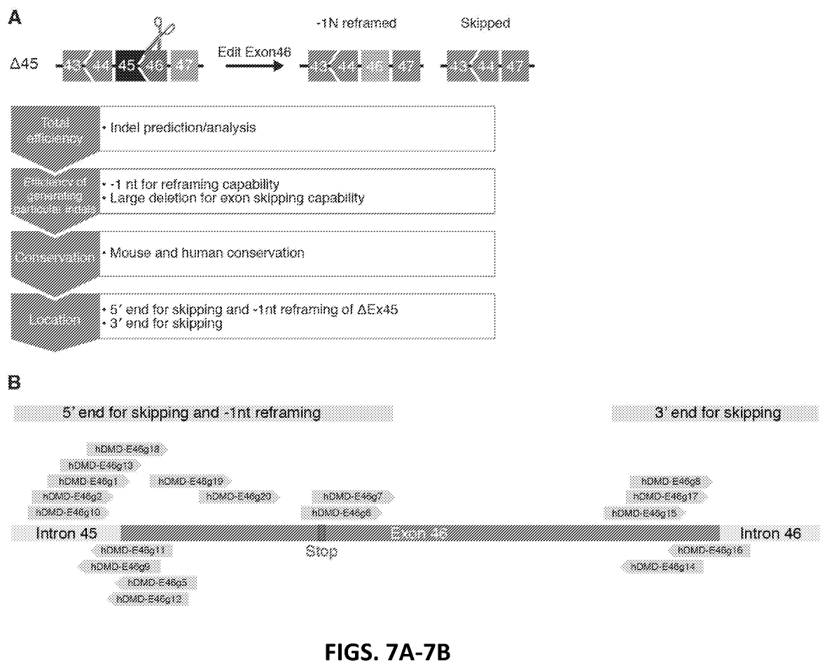

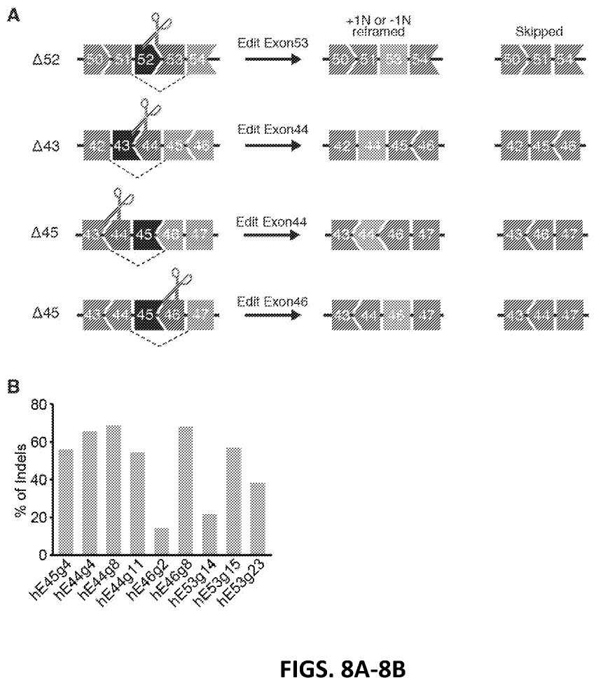

Identification of optimal sgRNAs for CRISPR/Cas9 correction of DMD exon 43, exon 45, and exon 52 deletions. Skipping or reframing of exon 44, exon 46, and exon 53 would apply to about 18% of DMD patients. To restore the ORF of the DMD patient with exon 43, exon 45, or exon 53 deletion, the inventors applied single guide RNA to disrupt the splicing junction of exon 44, exon 46, and exon 53 respectively, which results in reframing of the exon downstream of the deleted exon and restoration of the protein reading frame (FIGS. 2A, 4A, 5A, 6A, 7A, 8A). To test sgRNA efficiency within these regions, the inventors designed sgRNAs to target the region flanking splicing junctions of exon 44, exon 46 or exon 53 to reframe or skip the targeting exon (Table 2, Table 3, Table 8, Table 9, FIGS. 4B, 5B, 6B, 7B). The inventors validated the cleavage efficiency of these gRNAs in both mouse 10T1/2 or mouse N2a cells or human 293 cells. By T7E1 assay, the inventors demonstrated that exon 44 sgRNAs, exon 46 sgRNAs, and exon 53 sgRNAs efficiently cause DNA cleavage at Dmd exon target locus in mouse cells (FIG. 2B). By T7E1 assay, the inventors also demonstrated that exon 44 sgRNAs, exon 46 sgRNAs, and exon 53 sgRNAs efficiently cause DNA cleavage at DMD exon target locus in human cells (FIG. 2C). Additionally, by TIDE assay, the inventors demonstrated that 3 exon 44 sgRNAs (hDMD-E44g4, hDMD-E44g8, hDMD-E44g11), 2 exon 46 sgRNAs (hDMD-E46g2, hDMD-E46g8), and 3 exon 53 sgRNAs (hDMD-E53g14, hDMD-e53g15, hDMD-E53g23) efficiently cause DNA cleavage at a DMD exon target locus in human cells (FIG. 8B). hDMD-E45g4 was included as a positive control.

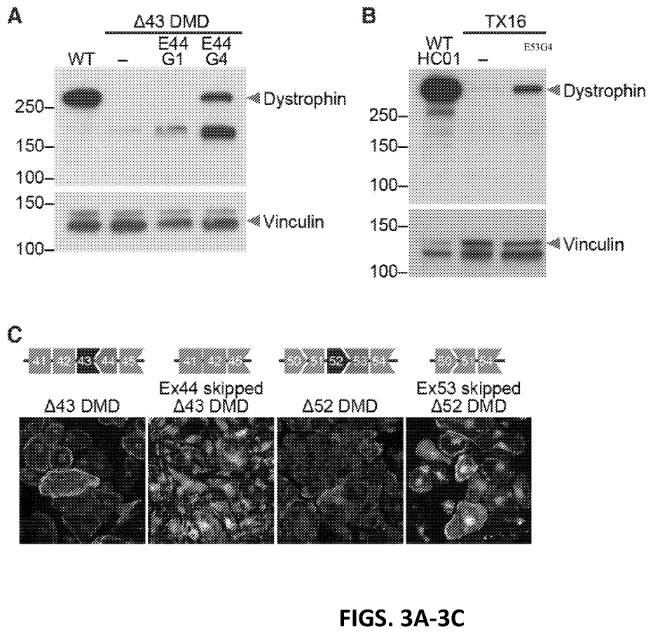

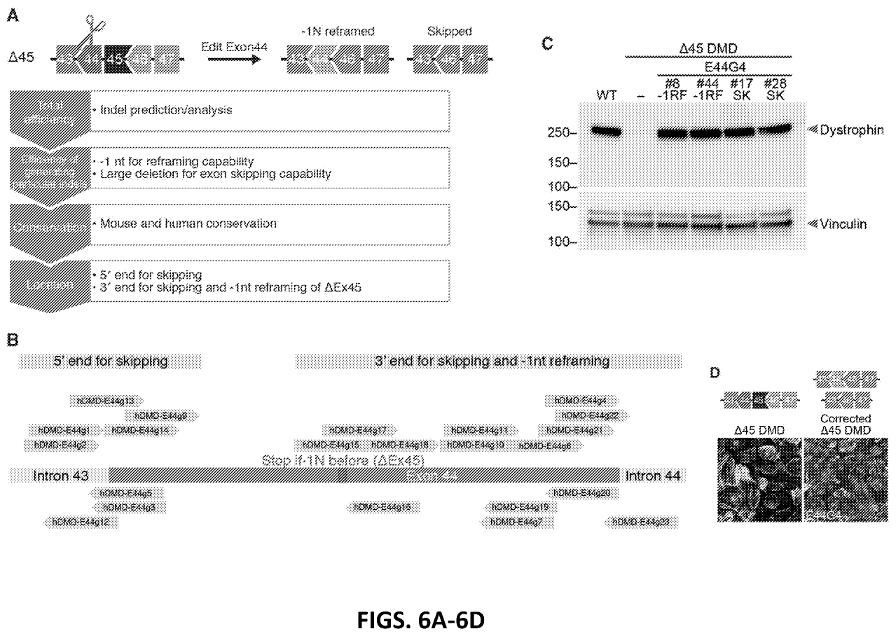

DMD iPSC-derived cardiomyocytes express dystrophin after CRISPR/Cas9 mediated genome editing by exon skipping and exon reframing. The inventors then generated iPSCs from DMD patients (TX16) that have deletion of exon 52 and an isogenic iPSC line with deletion of exon 43 (Δ43 DMD) and a deletion of exon 45 (Δ45 DMD). The inventors then tested exon 44 editing sgRNAs on Δ43 and Δ45 DMD iPSCs and showed restoration of dystrophin protein expression by Western blot analysis and immunostaining of iPSC-derived cardiomyocytes (FIGS. 3A, 3C, 6C, 6D). The inventors also tested exon 53 editing sgRNAs on TX16 patient derived iPSCs. The restoration of dystrophin in these TX16 DMD patient iPSCs was confirmed by Western blot analysis and immunostaining of iPSC-derived cardiomyocytes (FIG. 3B and FIG. 3C).

All of the compositions and/or methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and/or methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### VIII. REFERENCES

The following references, to the extent that they provide exemplary procedural or other details supplementary to those set forth herein, are specifically incorporated herein by reference.
Angel et al., Mol. Cell. Biol., 7:2256, 1987a.
Angel et al., Cell, 49:729, 1987b.
Baichwal and Sugden, In: Gene Transfer, Kucherlapati (Ed), NY, Plenum Press, 117-148, 1986.
Banerji et al., Cell, 27(2 Pt 1):299-308, 1981.
Banerji et al., Cell, 33(3):729-740, 1983.
Barnes et al., J. Biol. Chem., 272(17):11510-7, 1997.
Benvenisty and Neshif, Proc. Natl. Acad. Sci. USA, 83:9551-9555, 1986.
Berkhout et al., Cell, 59:273-282, 1989.
Bhaysar et al., Genomics, 35(1):11-23, 1996.
Bikard et al., Nucleic Acids Res. 41(15): 7429-7437, 2013.
Blanar et al., EMBO J., 8:1139, 1989.
Bodine and Ley, EMBO J., 6:2997, 1987.
Boshart et al., Cell, 41:521, 1985.
Bosze et al., EMBO J., 5(7):1615-1623, 1986.
Braddock et al., Cell, 58:269, 1989.
Brinster et al., Proc. Natl. Acad. Sci. USA, 82(13):4438-4442, 1985.
Bulla and Siddiqui, J. Virol., 62:1437, 1986.
Calcedo et al., J Infect Dis, 199(3): p. 381-90, 2009.
Campbell and Villarreal, Mol. Cell. Biol., 8:1993, 1988.
Campere and Tilghman, Genes and Dev., 3:537, 1989.
Campo et al., Nature, 303:77, 1983.
Celander and Haseltine, J. Virology, 61:269, 1987.
Celander et al., J. Virology, 62:1314, 1988.
Chandler et al., Cell, 33:489, 1983.
Chang et al., Mol. Cell. Biol., 9:2153, 1989.
Chang et al., Stem Cells., 27:1042-1049, 2009.
Chatterjee et al., Proc. Natl. Acad. Sci. USA, 86:9114, 1989.
Chen and Okayama, Mol. Cell Biol., 7:2745-2752, 1987.
Cho et al., Nat. Biotechnol. 31(3): 230-232, 2013.
Choi et al., Cell, 53:519, 1988.
Choi et al., Curr Gene Ther. 5(3); 299-310, 2005.
Coffin, In: Virology, Fields et al. (Eds.), Raven Press, NY, 1437-1500, 1990.
Cohen et al., J. Cell. Physiol., 5:75, 1987.
Costa et al., Mol. Cell. Biol., 8:81, 1988.
Couch et al., Am. Rev. Resp. Dis., 88:394-403, 1963.
Coupar et al., Gene, 68:1-10, 1988.
Cripe et al., EMBO J., 6:3745, 1987.
Culotta and Hamer, Mol. Cell. Biol., 9:1376, 1989.
Dandolo et al., J. Virology, 47:55-64, 1983.
De Villiers et al., Nature, 312(5991):242-246, 1984.
Deschamps et al., Science, 230:1174-1177, 1985.
Donnelly et al., J. Gen. Virol. 82, 1027-1041, 2001.
Dubensky et al., Proc. Natl. Acad. Sci. USA, 81:7529-7533, 1984.
Edbrooke et al., Mol. Cell. Biol., 9:1908, 1989.
Edlund et al., Science, 230:912-916, 1985.
EP 0273085
Fechheimer et al., Proc Natl. Acad. Sci. USA, 84:8463-8467, 1987.
Feng and Holland, Nature, 334:6178, 1988.
Ferkol et al., FASEB J., 7:1081-1091, 1993.
Firak et al., Mol. Cell. Biol., 6:3667, 1986.
Foecking et al., Gene, 45(1):101-105, 1986.
Fraley et al., Proc Natl. Acad. Sci. USA, 76:3348-3352, 1979
Franz et al., Cardoscience, 5(4):235-43, 1994.
Friedmann, Science, 244:1275-1281, 1989.
Fujita et al., Cell, 49:357, 1987.
Gao et al., J Virol, 78(12): p. 6381-8, 2004.
Ghosh and Bachhawat, In: Liver Diseases, Targeted Diagnosis and Therapy Using Specific Receptors and Ligands, Wu et al. (Eds.), Marcel Dekker, NY, 87-104, 1991.
Ghosh-Choudhury et al., EMBO J., 6:1733-1739, 1987.
Gilles et al., Cell, 33:717, 1983.
Gloss et al., EMBO J., 6:3735, 1987.
Godbout et al., Mol. Cell. Biol., 8:1169, 1988.
Gomez-Foix et al., J. Biol. Chem., 267:25129-25134, 1992.
Goodbourn and Maniatis, Proc. Natl. Acad. Sci. USA, 85:1447, 1988.
Goodbourn et al., Cell, 45:601, 1986.
Gopal, Mol. Cell Biol., 5:1188-1190, 1985.
Gopal-Srivastava et al., J. Mol. Cell. Biol., 15(12):7081-90, 1995.
Graham and Prevec, In: Methods in Molecular Biology: Gene Transfer and Expression Protocol, Murray (Ed.), Humana Press, Clifton, N.J., 7:109-128, 1991.
Graham and van der Eb, Virology, 52:456-467, 1973.
Graham et al., J. Gen. Virol., 36:59-72, 1977.
Greene et al., Immunology Today, 10:272, 1989
Grosschedl and Baltimore, Cell, 41:885, 1985.
Grunhaus and Horwitz, Seminar in Virology, 3:237-252, 1992.
Harland and Weintraub, J. Cell Biol., 101:1094-1099, 1985.
Haslinger and Karin, Proc. Natl. Acad. Sci. USA, 82:8572, 1985.
Hauber and Cullen, J. Virology, 62:673, 1988.
Hen et al., Nature, 321:249, 1986.
Hensel et al., Lymphokine Res., 8:347, 1989.
Hermonat and Muzycska, Proc. Nat'l Acad. Sci. USA, 81:6466-6470, 1984.
Herr and Clarke, Cell, 45:461, 1986.
Hersdorffer et al., DNA Cell Biol., 9:713-723, 1990.
Herz and Gerard, Proc. Nat'l. Acad. Sci. USA 90:2812-2816, 1993.
Hirochika et al., J. Virol., 61:2599, 1987.
Holbrook et al., Virology, 157:211, 1987.
Horlick and Benfield, Mol. Cell. Biol., 9:2396, 1989.
Horwich et al., J. Virol., 64:642-650, 1990.
Hsu et al., Natl Biotechnol. 31:827-832, 2013
Huang et al., Cell, 27:245, 1981.
Hug et al., Mol. Cell. Biol., 8:3065, 1988.
Hwang et al., Mol. Cell. Biol., 10:585, 1990.
Imagawa et al., Cell, 51:251, 1987.
Imbra and Karin, Nature, 323:555, 1986.
Imler et al., Mol. Cell. Biol., 7:2558, 1987.
Imperiale and Nevins, Mol. Cell. Biol., 4:875, 1984.
Jakobovits et al., Mol. Cell. Biol., 8:2555, 1988.
Jameel and Siddiqui, Mol. Cell. Biol., 6:710, 1986.
Jaynes et al., Mol. Cell. Biol., 8:62, 1988.
Jinek et al., Science 337, 816-821, 2012.
Johnson et al., Mol. Cell. Biol., 9:3393, 1989.
Jones and Shenk, Cell, 13:181-188, 1978.
Kadesch and Berg, Mol. Cell. Biol., 6:2593, 1986.
Kaneda et al., Science, 243:375-378, 1989.
Karin et al., Mol. Cell. Biol., 7:606, 1987.
Karlsson et al., EMBO J., 5:2377-2385, 1986.
Katinka et al., Cell, 20:393, 1980.
Kato et al., J Biol Chem., 266(6):3361-3364, 1991.
Kawamoto et al., Mol. Cell. Biol., 8:267, 1988.
Kelly et al., J. Cell Biol., 129(2):383-96, 1995.
Kiledjian et al., Mol. Cell. Biol., 8:145, 1988.
Kimura et al., Dev. Growth Differ., 39(3):257-65, 1997.
Klamut et al., Mol. Cell. Biol., 10:193, 1990.
Klein et al., Nature, 327:70-73, 1987.
Koch et al., Mol. Cell. Biol., 9:303, 1989.
Kriegler and Botchan, In: Eukaryotic Viral Vectors, Gluzman (Ed.), Cold Spring Harbor: Cold Spring Harbor Laboratory, NY, 1982.
Kriegler and Botchan, Mol. Cell. Biol., 3:325, 1983.
Kriegler et al., Cell, 38:483, 1984.
Kriegler et al., Cell, 53:45, 1988.
Kuhl et al., Cell, 50:1057, 1987.
Kunz et al., Nucl. Acids Res., 17:1121, 1989.
LaPointe et al., Hypertension, 27(3):715-22, 1996
LaPointe et al., J. Biol. Chem., 263(19):9075-8, 1988.
Larsen et al., Proc. Natl. Acad. Sci. USA., 83:8283, 1986.
Laspia et al., Cell, 59:283, 1989.
Latimer et al., Mol. Cell. Biol., 10:760, 1990.
Le Gal La Salle et al., Science, 259:988-990, 1993.
Lee et al., Nature, 294:228, 1981.
Levinson et al., Nature, 295:79, 1982.
Levrero et al., Gene, 101:195-202, 1991.
Lin et al., Mol. Cell. Biol., 10:850, 1990.
Luria et al., EMBO J., 6:3307, 1987.
Lusky and Botchan, Proc. Natl. Acad. Sci. USA, 83:3609, 1986.
Lusky et al., Mol. Cell. Biol., 3:1108, 1983.
Majors and Varmus, Proc. Natl. Acad. Sci. USA, 80:5866, 1983.
Mali et al., Science 339, 823-826, 2013a.
Mali et al., Nat Methods 10, 957-963, 2013b.
Mali et al., Nat. Biotechnol. 31:833-838, 2013c.
Mann et al., Cell, 33:153-159, 1983.
Markowitz et al., J. Virol., 62:1120-1124, 1988.
McNeall et al., Gene, 76:81, 1989.
Melnick et al., J Bacteriol, 90(1): p. 271-4, 1965.
Miksicek et al., Cell, 46:203, 1986.
Mordacq and Linzer, Genes and Dev., 3:760, 1989.
Moreau et al., Nucl. Acids Res., 9:6047, 1981.
Moss et al., J. Gen. Physiol., 108(6):473-84, 1996.
Muesing et al., Cell, 48:691, 1987.
Nathwani et al., New Engl. J. Med. 365:2357-2365, 2011.
Nicolas and Rubinstein, In: Vectors: A survey of molecular cloning vectors and their uses, Rodriguez and Denhardt (Eds.), Stoneham: Butterworth, 494-513, 1988.
Nicolau and Sene, Biochim. Biophys. Acta, 721:185-190, 1982.
Nicolau et al., Methods Enzymol., 149:157-176, 1987.
Ondek et al., EMBO J., 6:1017, 1987.
Ornitz et al., Mol. Cell. Biol., 7:3466, 1987.
Palmiter et al., Cell, 29:701, 1982a.
Palmiter et al., Nature, 300:611, 1982b.
Paskind et al., Virology, 67:242-248, 1975.
Pech et al., Mol. Cell. Biol., 9:396, 1989.
Perales et al., Proc. Natl. Acad. Sci. USA, 91(9):4086-4090, 1994.
Perez-Stable and Constantin, Mol. Cell. Biol., 10:1116, 1990.
Picard et al., Nature, 307:83, 1984.
Pinkert et al., Genes and Dev., 1:268, 1987.
Ponta et al., Proc. Natl. Acad. Sci. USA, 82:1020, 1985.
Porton et al., Mol. Cell. Biol., 10:1076, 1990.
Potter et al., Proc. Natl. Acad. Sci. USA, 81:7161-7165, 1984.
Queen and Baltimore, Cell, 35:741, 1983.
Quinn et al., Mol. Cell. Biol., 9:4713, 1989.
Racher et al., Biotech. Techniques, 9:169-174, 1995.
Ragot et al., Nature, 361:647-650, 1993.
Redondo et al., Science, 247:1225, 1990.
Reisman and Rotter, Mol. Cell. Biol., 9:3571, 1989.
Renan, Radiother. Oncol., 19:197-218, 1990.
Resendez Jr. et al., Mol. Cell. Biol., 8:4579, 1988.
Rich et al., Hum. Gene Ther., 4:461-476, 1993.
Ridgeway, In: Vectors: A survey of molecular cloning vectors and their uses, Stoneham: Butterworth, 467-492, 1988.
Ripe et al., Mol. Cell. Biol., 9:2224, 1989.
Rippe et al., Mol. Cell Biol., 10:689-695, 1990.
Riffling et al., Nuc. Acids Res., 17:1619, 1989.
Rosen et al., Cell, 41:813, 1988.
Rosenfeld et al., Cell, 68:143-155, 1992.
Rosenfeld et al., Science, 252:431-434, 1991.
Roux et al., Proc. Natl. Acad. Sci. USA, 86:9079-9083, 1989.
Sambrook and Russell, Molecular Cloning: A Laboratory Manual 3^{rd} Ed., Cold Spring Harbor Laboratory Press, 2001.
Sakai et al., Genes and Dev., 2:1144, 1988.
Satake et al., J. Virology, 62:970, 1988.
Schaffner et al., J. Mol. Biol., 201:81, 1988.
Searle et al., Mol. Cell. Biol., 5:1480, 1985.
Sharp et al., Cell, 59:229, 1989.
Shaul and Ben-Levy, EMBO J., 6:1913, 1987.
Sherman et al., Mol. Cell. Biol., 9:50, 1989.
Sleigh et al., J. EMBO, 4:3831, 1985.
Spalholz et al., Cell, 42:183, 1985.
Spandau and Lee, J. Virology, 62:427, 1988.
Spandidos and Wilkie, EMBO J., 2:1193, 1983.
Stephens and Hentschel, Biochem. 1, 248:1, 1987.
Stratford-Perricaudet and Perricaudet, In: Human Gene Transfer, Cohen-Haguenauer and Boiron (Eds.), John Libbey Eurotext, France, 51-61, 1991.
Stratford-Perricaudet et al., Hum. Gene. Ther., 1:241-256, 1990.
Stuart et al., Nature, 317:828, 1985.
Sullivan and Peterlin, Mol. Cell. Biol., 7:3315, 1987.
Swartzendruber and Lehman, J. Cell. Physiology, 85:179, 1975.
Takebe et al., Mol. Cell. Biol., 8:466, 1988.
Tavernier et al., Nature, 301:634, 1983.
Taylor and Kingston, Mol. Cell. Biol., 10:165, 1990a.
Taylor and Kingston, Mol. Cell. Biol., 10:176, 1990b.
Taylor et al., J. Biol. Chem., 264:15160, 1989.
Temin, In: Gene Transfer, Kucherlapati (Ed.), NY, Plenum Press, 149-188, 1986.
Thiesen et al., J. Virology, 62:614, 1988.
Top et al., J. Infect. Dis., 124:155-160, 1971.
Tronche et al., Mol. Biol. Med., 7:173, 1990.
Trudel and Constantini, Genes and Dev. 6:954, 1987.
Tur-Kaspa et al., Mol. Cell Biol., 6:716-718, 1986.
Tyndell et al., Nuc. Acids. Res., 9:6231, 1981.
Vannice and Levinson, J. Virology, 62:1305, 1988.
Varmus et al., Cell, 25:23-36, 1981.
Vasseur et al., Proc Natl. Acad. Sci. USA., 77:1068, 1980.
Wagner et al., Proc. Natl. Acad. Sci. USA, 87(9):3410-3414, 1990.
Wang and Calame, Cell, 47:241, 1986.
Wang et al., Cell, 153:910-910, 2013.
Weber et al., Cell, 36:983, 1984.
Weinberger et al. Mol. Cell. Biol., 8:988, 1984.
Winoto et al., Cell, 59:649, 1989.
Wong et al., Gene, 10:87-94, 1980.
Wu and Wu, Adv. Drug Delivery Rev., 12:159-167, 1993.
Wu and Wu, Biochemistry, 27:887-892, 1988.
Wu and Wu, J. Biol. Chem., 262:4429-4432, 1987.
Wu et al., Molecular Therapy 14(3), 316-327, 2006.
Wu et al., Cell Stem Cell 13, 659-662, 2013.
Wu et al., Nat Biotechnol 32, 670-676, 2014.
Yamauchi-Takihara et al., Proc. Natl. Acad. Sci. USA, 86(10):3504-3508, 1989.
Yang et al., Proc. Natl. Acad. Sci. USA, 87:9568-9572, 1990.
Yutzey et al. Mol. Cell. Biol., 9:1397, 1989.
Zelenin et al., FEBS Lett., 280:94-96, 1991.
Ziober and Kramer, J. Bio. Chem., 271(37):22915-22922, 1996.

1. A nucleic acid comprising:
a sequence encoding a single guide RNA (sgRNA) comprising a spacer sequence and a scaffold sequence;
wherein the spacer sequence comprises the sequence of any one of SEQ ID NOs: 135-146, 170-260, 337-339, or 617.

2. The nucleic acid of claim 1, wherein the scaffold sequence comprises the sequence of any one of SEQ ID NO: 147-153.

3. The nucleic acid of claim 1, wherein the nucleic acid comprises one copy of the sequence encoding the sgRNA.

4. The nucleic acid of claim 1, wherein the nucleic acid comprises two, three, four, or five copies of the sequence encoding the sgRNA.

5. The nucleic acid of claim 1, wherein the nucleic acid comprises a sequence encoding a promoter, wherein the promoter drives expression of the sgRNA.

6. The nucleic acid of claim 5, wherein the nucleic acid comprises three copies of the sequence encoding the sgRNA, wherein the nucleic acid comprises a sequence encoding a first promoter and expression of the first copy of the sgRNA is driven by the first promoter, wherein the nucleic acid comprises a sequence encoding a second promoter and expression of the second copy of the sgRNA is driven by the second promoter, and wherein the nucleic acid comprises a sequence encoding a third promoter and expression of the third copy of the sgRNA is driven by the third promoter.

7. The nucleic acid of claim 1, wherein the nucleic acid further comprises a sequence encoding a nuclease.

8. The nucleic acid of claim 7, wherein the nuclease is a Type II, Type V-A, Type V-B, Type V-C, Type V-U, or Type VI-B nuclease.

9. The nucleic acid of claim 7, wherein the nuclease is a TAL nuclease, a meganuclease, or a zinc-finger nuclease.

10. The nucleic acid of claim 7, wherein the nuclease is a Cas9, Cas12a, Cas12b, Cas12c, Tnp-B like, Cas13a (C2c2), Cas13b, or Cas14 nuclease.

11. The nucleic acid of claim 10, wherein the nuclease is a Cas9 nuclease.

12. The nucleic acid of claim 11, wherein the Cas9 is a Streptococcus pyogenes or Streptococcus aureus Cas9.

13. The nucleic acid of claim 11, wherein the nuclease is a modified Cas9 nuclease.

14. The nucleic acid of claim 12, wherein the nuclease is a modified Streptococcus pyogenes Cas9 or a modified Streptococcus aureus Cas9.

15. A recombinant vector comprising the nucleic acid of claim 1.

16. The recombinant vector of claim 15, wherein the recombinant vector is a plasmid.

17. The recombinant vector of claim 15, wherein the recombinant vector is an expression vector.

18. The recombinant vector of claim 15, wherein the recombinant vector is a viral vector.

19. The recombinant vector of claim 18, wherein the viral vector is a lentiviral vector, a retroviral vector, an adenoviral vector, or an adeno-associated virus (AAV) vector.

20. The recombinant vector of claim 19, wherein the viral vector is an adeno-associated virus (AAV) vector.

21. The recombinant vector of claim 20, wherein the serotype of the AAV vector is selected from AAV1, AAV2, AAV3, AAV4, AAV5, AAV6, AAV7, AAV8, AAV9, AAV10, AAV11, AAV12, AAVRh74, AAV2i8, AAVRh10, AAV39, AAV43, AAVRh8, avian AAV, bovine AAV, canine AAV, equine AAV, and ovine AAV.

22. The recombinant vector of claim 20, wherein the AAV vector is replication-defective or conditionally replication defective.

23. The recombinant vector of claim 21, wherein the serotype of the AAV vector is AAV9.

24. A non-viral vector comprising the nucleic acid of claim 1, wherein the non-viral vector comprises calcium phosphate, liposomes, nanoparticles, and/or lipid emulsions.

25. An AAV expression cassette comprising:
a first inverted terminal repeat (ITR);
a first promoter;
the nucleic acid of claim 1; and
a second ITR.

26. The AAV expression cassette of claim 25, wherein the AAV expression cassette further comprises a polyadenosine (polyA) sequence.

27. The AAV expression cassette of claim 25, wherein one or both of the first ITR and the second ITR are isolated or derived from any one of AAV1, AAV2, AAV3, AAV4, AAV5, AAV6, AAV7, AAV8, AAV9, AAV10, AAV11, AAV12, AAVRh74, AAV2i8, AAVRh10, AAV39, AAV43, AAVRh8, avian AAV, bovine AAV, canine AAV, equine AAV, or ovine AAV.

28. The AAV expression cassette of claim 25.

29. An AAV vector comprising the nucleic acid of claim 1.

30. The AAV vector of claim 28, wherein the AAV vector has the serotype of AAV1, AAV2, AAV3, AAV4, AAV5, AAV6, AAV7, AAV8, AAV9, AAV10, AAV11, AAV12, AAVRh74, AAV2i8, AAVRh10, AAV39, AAV43, AAVRh8, avian AAV, bovine AAV, canine AAV, equine AAV, or ovine AAV.

31. The AAV vector of claim 29, wherein the AAV vector is replication-defective or conditionally replication defective.

32. The AAV vector of claim 30, wherein the serotype of the AAV vector is AAV9.

33. A composition comprising the nucleic acid of claim 1.

34. The composition of claim 33, further comprising a pharmaceutically acceptable carrier.

35. A cell comprising the nucleic acid of claim 1.

36. The cell of claim 35, wherein the cell is a stem cell.

37. The cell of claim 35, wherein the cell is a mammalian cell.

38. The cell of claim 37, wherein the cell is a human cell.

39. A composition comprising the AAV vector of claim 29.

40. The composition of claim 39, further comprising a pharmaceutically acceptable carrier.

41. A method of correcting a gene defect in a cell, the method comprising contacting the cell with:
the nucleic acid of claim 1.

42. The method of claim 41, wherein the cell is a stem cell.

43. The method of claim 41, wherein the cell is a mammalian cell.

44. The method of claim 43, wherein the cell is a human cell.

45. A method of treating a subject suffering from Duchenne muscular dystrophy, the method comprising administering to the subject a therapeutically effective amount of:
the nucleic acid of claim 1.

46. A method of treating a subject suffering from Duchenne muscular dystrophy, the method comprising administering to the subject:
a first vector, wherein the first vector is the recombinant vector of claim 1, and
a second vector, wherein the second vector encodes a nuclease.

47. The method of claim 46, wherein the nuclease is a Type II, Type V-A, Type V-B, Type V-C, Type V-U, or Type VI-B nuclease.

48. The method of claim 46, wherein the nuclease is a TAL nuclease, a meganuclease, or a zinc-finger nuclease.

49. The method of claim 46, wherein the nuclease is a Cas9, Cas12a, Cas12b, Cas12c, Tnp-B like, Cas13a (C2c2), Cas13b, or Cas14 nuclease.

50. The method of claim 49, wherein the nuclease is a Cas9 nuclease.

51. The method of claim 50, wherein the Cas9 is a Streptococcus pyogenes or Streptococcus aureus Cas9.

52. The method of claim 50, wherein the nuclease is a modified Cas9 nuclease.

53. The method of claim 52, wherein the nuclease is a modified Streptococcus pyogenes Cas9 or a modified Streptococcus aureus Cas9.

54. The method of claim 46, wherein the second vector is a plasmid.

55. The method of claim 46, wherein the second vector is an expression vector.

56. The method of claim 46, wherein the second vector is a viral vector.

57. The method of claim 56, wherein the viral vector is a lentiviral vector, a retroviral vector, an adenoviral vector, or an adeno-associated virus (AAV) vector.

58. The method of claim 57, wherein the viral vector is an adeno-associated virus (AAV) vector.

59. The method of claim 58, wherein the serotype of the AAV vector is selected from AAV1, AAV2, AAV3, AAV4, AAV5, AAV6, AAV7, AAV8, AAV9, AAV10, AAV11, AAV12, AAVRh74, AAV2i8, AAVRh10, AAV39, AAV43, AAVRh8, avian AAV, bovine AAV, canine AAV, equine AAV, and ovine AAV.

60. The method of claim 46, wherein the second vector is a non-viral vector, wherein the non-viral vector comprises calcium phosphate, liposomes, nanoparticles, and/or lipid emulsions.

61. The method of claim 46, wherein the administering induces a frameshift mutation in a target nucleic acid sequence in a cell of the patient.

62. The method of claim 61, wherein the frameshift mutation comprises a deletion of at least one nucleotide, wherein the number of nucleotides deleted is not a multiple of 3.

63. The method of claim 62, wherein the frameshift mutation comprises a deletion of 1, 2, 4, 5, 7, 8, 10, 11, 13, 14, 16, 17, 19 or 20 nucleotides.

64. The method of claim 61, wherein the frameshift mutation comprises an insertion of at least one nucleotide, wherein the number of nucleotides inserted is not a multiple of 3.

65. The method of claim 64, wherein the frameshift mutation comprises an insertion of 1, 2, 4, 5, 7, 8, 10, 11, 13, 14, 16, 17, 19 or 20 nucleotides.

66. The method of claim 65, wherein the frameshift mutation comprises an insertion of 1 nucleotide.

67. The method of claim 46, wherein the first vector and the second vector are administered simultaneously.

68. The method of claim 46, wherein the first vector and the second vector are administered sequentially.

69. The method of claim 46, wherein the first vector and the second vector are administered locally.

70. The method of claim 46, wherein the first vector and the second vector are administered systemically.

71. The method of claim 46, wherein the first vector and the second vector are administered by an oral, rectal, transmucosal, topical, transdermal, inhalation, intravenous, subcutaneous, intradermal, intramuscular, intra-articular, intrathecal, intraventricular, intravenous, intraperitoneal, intranasal, or intraocular route of administration.

72. The method of claim 46, wherein the subject is greater than or equal to 18 years old.

73. The method of claim 46, wherein the subject is less than 18 years old.

74. The method of claim 73, wherein the subject is less than 2 years old.

75. The method of claim 46, wherein the subject is a human.

76. The method of claim 46, wherein the ratio of the first vector to the second vector is 1:1 to 1:100.

77. The method of claim 46, wherein the ratio of the second vector to the first vector is 1:1 to 1:100.

78. A combination therapy comprising;
a first composition comprising a first vector comprising the nucleic acid of claim 1; and
a second composition comprising a second vector comprising a nucleic acid that encodes a nuclease.

79. The combination therapy of claim 78, wherein at least one of the first and the second composition comprises a pharmaceutically acceptable carrier.

80. The combination therapy of claim 78, wherein the nuclease is a Type II, Type V-A, Type V-B, Type V-C, Type V-U, or Type VI-B nuclease.

81. The combination therapy of claim 78, wherein the nuclease is a TAL nuclease, a meganuclease, or a zinc-finger nuclease.

82. The combination therapy of claim 78, wherein the nuclease is a Cas9, Cas12a, Cas12b, Cas12c, Tnp-B like, Cas13a (C2c2), Cas13b, or Cas14 nuclease.

83. The combination therapy of claim 82, wherein the nuclease is a Cas9 nuclease.

84. The combination therapy of claim 83, wherein the Cas9 is a Streptococcus pyogenes or Streptococcus aureus Cas9.

85. The combination therapy of claim 83, wherein the nuclease is a modified Cas9 nuclease.

86. The combination therapy of claim 85, wherein the nuclease is a modified Streptococcus pyogenes Cas9 or a modified Streptococcus aureus Cas9.

87. The composition of claim 33.

88. The composition of claim 33.

89. A mouse whose genome comprises (a) a deletion of exon 43 of the dystrophin gene resulting in an out of frame shift and a premature stop codon in exon 44, (b) a deletion of exon 45 resulting in an out of frame shift and premature stop codon in exon 46, or (c) a deletion of exon 52 resulting in an out of frame shift and premature stop codon in exon 53.

90. The mouse of claim 89, further comprising a reporter gene located downstream of and in frame with exon 79 of the dystrophin gene, and upstream of a dystrophin 3′-UTR, wherein said reporter gene is expressed when exon 79 is translated in frame with exon 45, exon 47, or exon 54.

91. The mouse of claim 90, wherein the reporter gene is luciferase.

92. The mouse of claim 90, further comprising a protease coding sequence upstream of and in frame with said reporter gene, and downstream of and in frame with exon 79.

93. The mouse of claim 92, wherein said protease is autocatalytic.

94. The mouse of claim 93, wherein said protease is 2A protease.

95. The mouse of claim 89, wherein the mouse is heterozygous for said deletion.

96. The mouse of claim 89, wherein the mouse is homozygous for said deletion.

97. The mouse of claim 89, wherein the mouse exhibits increased creatine kinase levels.

98. The mouse of claim 89, wherein the mouse does not exhibit detectable dystrophin protein in heart or skeletal muscle.

99. A method of producing the mouse of claim 89 comprising:
(a1) contacting a fertilized oocyte with CRISPR/Cas9 elements and two single guide RNA (sgRNA) targeting sequences flanking exon 43, thereby creating a modified oocyte, wherein deletion of exon 43 by CRISPR/Ca9 results in an out of frame shift and a premature stop codon in exon 44;
(a2) contacting a fertilized oocyte with CRISPR/Cas9 elements and two single guide RNA (sgRNA) targeting sequences flanking exon 45, thereby creating a modified oocyte, wherein deletion of exon 45 by CRISPR/Ca9 results in an out of frame shift and a premature stop codon in exon 46; or
(a3) contacting a fertilized oocyte with CRISPR/Cas9 elements and two single guide RNA (sgRNA) targeting sequences flanking exon 52, thereby creating a modified oocyte, wherein deletion of exon 52 by CRISPR/Ca9 results in an out of frame shift and a premature stop codon in exon 53; and
(b) transferring said modified oocyte into a recipient female.

100. The method of claim 99, wherein said oocyte comprises a dystrophin gene having a reporter gene located downstream of and in frame with exon 79 of said dystrophin gene, and upstream of a dystrophin 3′-UTR, wherein said reporter gene is expressed when exon 79 is translated in frame with exon 45, exon 47, or exon 54.

101. The method of claim 100, wherein the reporter gene is luciferase.

102. The method of claim 100, further comprising a protease coding sequence upstream of and in frame with said reporter gene, and downstream of and in frame with exon 79.

103. The method of claim 102, wherein said protease is autocatalytic.

104. The method of claim 103, wherein said protease is 2A protease.

105. The method of claim 99, wherein the mouse is heterozygous for said deletion.

106. The method of claim 99, wherein the mouse is homozygous for said deletion.

107. The method of claim 99, wherein the mouse exhibits increased creatine kinase levels.

108. The method of claim 99, wherein the mouse does not exhibit detectable dystrophin protein in heart or skeletal muscle.

109. An isolated cell obtained from the mouse of claim 89.

110. The isolated cell of claim 109, further comprising a reporter gene located downstream of and in frame with exon 79 of the dystrophin gene, and upstream of a dystrophin 3′-UTR, wherein said reporter gene is expressed when exon 79 is translated in frame with exon 45, exon 47, or exon 54.

111. The isolated cell of claim 110, wherein the reporter gene is luciferase.

112. The mouse of claim 110, further comprising a protease coding sequence upstream of and in frame with said reporter gene, and downstream of and in frame with exon 79.

113. The cell of claim 112, wherein said protease is autocatalytic.

114. The cell of claim 113, wherein said protease is 2A protease.

115. The cell of claim 109, wherein the cell is heterozygous for said deletion.

116. The cell of claim 109, wherein the cell is homozygous for said deletion.

117. A mouse produced by a method comprising the steps of:
(a1) contacting a fertilized oocyte with CRISPR/Cas9 elements and two single guide RNA (sgRNA) targeting sequences flanking exon 43, thereby creating a modified oocyte, wherein deletion of exon 43 by CRISPR/Ca9 results in an out of frame shift and a premature stop codon in exon 44;
(a2) contacting a fertilized oocyte with CRISPR/Cas9 elements and two single guide RNA (sgRNA) targeting sequences flanking exon 45, thereby creating a modified oocyte, wherein deletion of exon 45 by CRISPR/Ca9 results in an out of frame shift and a premature stop codon in exon 46; or
(a3) contacting a fertilized oocyte with CRISPR/Cas9 elements and two single guide RNA (sgRNA) targeting sequences flanking exon 52, thereby creating a modified oocyte, wherein deletion of exon 52 by CRISPR/Ca9 results in an out of frame shift and a premature stop codon in exon 53; and
(b) transferring said modified oocyte into a recipient female.

118. A method of screening a candidate substance for DMD exon-skipping activity comprising:
(a) contacting a mouse according to claim 1 with a candidate substance; and
(b) assessing in frame transcription and/or translation of exon 79,
wherein the presence of in frame transcription and/or translation of exon 79 indicates said candidate substance exhibits exon-skipping activity.

119. The method of claim 118, wherein the mouse does not exhibit detectable dystrophin protein in heart or skeletal muscle.

120. The method of claim 118, wherein the genome of the mouse further comprises a reporter gene located downstream of and in frame with exon 79 of the dystrophin gene, and upstream of a dystrophin 3′-UTR, wherein said reporter gene is expressed when exon 79 is translated in frame with exon 45, exon 47, or exon 54.

121. The method of claim 120, wherein the reporter gene is luciferase.

122. The method of claim 120, wherein the genome of the mouse further comprises a protease coding sequence upstream of and in frame with said reporter gene, and downstream of and in frame with exon 79.

123. The method of claim 122, wherein said protease is autocatalytic.

124. The method of claim 123, wherein said protease is 2A protease.

125. The method of claim 118, wherein the mouse is heterozygous for said deletion.

126. The method of claim 118, wherein the mouse is homozygous for said deletion.

127. The method of claim 118, wherein the mouse exhibits increased creatine kinase levels.

128. An isolated nucleic acid comprising a sequence of any one of SEQ ID NO: 1-72, 340-359, or 360-515.

129. A double-stranded nucleic acid formed by hybridization of SEQ ID NO: 1 and 2, SEQ ID NO: 3 and 4, SEQ ID NO: 5 and 6, SEQ ID NO: 7 and 8, SEQ ID NO: 9 and 10, SEQ ID NO: 11 and 12, SEQ ID NO: 13 and 14, SEQ ID NO: 15 and 16, SEQ ID NO: 17 and 18, SEQ ID NO: 19 and 20, SEQ ID NO: 21 and 22, SEQ ID NO: 23 and 24, SEQ ID NO: 25 and 26, SEQ ID NO: 27 and 28, SEQ ID NO: 29 and 30, SEQ ID NO: 31 and 32, SEQ ID NO: 33 and 34, SEQ ID NO: 35 and 36, SEQ ID NO: 37 and 38, SEQ ID NO: 39 and 40, SEQ ID NO: 41 and 42, SEQ ID NO: 43 and 44, SEQ ID NO: 45 and 46, SEQ ID NO: 47 and 48, SEQ ID NO: 49 and 50, SEQ ID NO: 51 and 52, SEQ ID NO: 53 and 54, SEQ ID NO: 55 and 56, SEQ ID NO: 57 and 58, SEQ ID NO: 59 and 60, SEQ ID NO: 61 and 62, SEQ ID NO: 63 and 64, SEQ ID NO: 65 and 66, SEQ ID NO: 67 and 68, SEQ ID NO: 69 and 70, and SEQ ID NO: 71 and 72.

130. An expression construct comprising a nucleic acid formed by hybridization of SEQ ID NO: 1 and 2, SEQ ID NO: 3 and 4, SEQ ID NO: 5 and 6, SEQ ID NO: 7 and 8, SEQ ID NO: 9 and 10, SEQ ID NO: 11 and 12, SEQ ID NO: 13 and 14, SEQ ID NO: 15 and 16, SEQ ID NO: 17 and 18, SEQ ID NO: 19 and 20, SEQ ID NO: 21 and 22, SEQ ID NO: 23 and 24, SEQ ID NO: 25 and 26, SEQ ID NO: 27 and 28, SEQ ID NO: 29 and 30, SEQ ID NO: 31 and 32, SEQ ID NO: 33 and 34, SEQ ID NO: 35 and 36, SEQ ID NO: 37 and 38, SEQ ID NO: 39 and 40, SEQ ID NO: 41 and 42, SEQ ID NO: 43 and 44, SEQ ID NO: 45 and 46, SEQ ID NO: 47 and 48, SEQ ID NO: 49 and 50, SEQ ID NO: 51 and 52, SEQ ID NO: 53 and 54, SEQ ID NO: 55 and 56, SEQ ID NO: 57 and 58, SEQ ID NO: 59 and 60, SEQ ID NO: 61 and 62, SEQ ID NO: 63 and 64, SEQ ID NO: 65 and 66, SEQ ID NO: 67 and 68, SEQ ID NO: 69 and 70, and SEQ ID NO: 71 and 72.

131. The expression construct of claim 130, wherein said expression construct is a viral vector.

132. A kit comprising one or more isolated nucleic acids of any one of SEQ ID NO: 1-72, 340-359, or 360-515.

133. A method of correcting a dystrophin gene defect in exon 44, exon 46 or exon 53 of the DMD gene in a subject comprising contacting a cell in said subject with Cpf1 or Cas9 and a DMD guide RNA as defined in claim 42, resulting in selective skipping of a mutant DMD exon.

134. The method of claim 133, wherein said cell is a muscle cell, a satellite cell, or an iPSC or iPSC-CM.

135. The method of claim 133, wherein Cas9, Cpf1 and/or DMD guide RNA are provided to said cell through expression from one or more expression vectors coding therefor.

136. The method of claim 135, wherein said expression vector is a viral vector.

137. The method of claim 136, wherein said viral vector is an adeno-associated viral vector.

138. The method of claim 135, wherein said expression vector is a non-viral vector.

139. The method of claim 133, wherein Cas9, Cpf1 or Cas9 is provided to said cell as naked plasmid DNA or chemically-modified mRNA.

140. The method of claim 133, further comprising contacting said cell with a single-stranded DMD oligonucleotide to effect homology directed repair.

141. The method of claim 133, wherein Cpf1 or Cas9, DMD guide RNA and/or single-stranded DMD oligonucleotide, or expression vectors coding therefor, are provided to said cell in one or more nanoparticles.

142. The method of claim 133, wherein said Cpf1 or Cas9, DMD guide RNA and/or single-stranded DMD oligonucleotide are delivered directly to a muscle tissue.

143. The method of claim 142, wherein said muscle tissue is tibialis anterior, quadricep, soleus, diaphragm or heart.

144. The method of claim 133, wherein said Cpf1 or Cas9, DMD guide RNA and/or single-stranded DMD oligonucleotide are delivered systemically.

145. The method of claim 133, wherein said subject exhibits normal dystrophin-positive myofibers and/or mosaic dystrophin-positive myofibers containing centralized nuclei.

146. The method of claim 133, wherein said subject exhibits a decreased serum CK level as compared to a serum CK level prior to contacting.

147. The method of claim 133, wherein said subject exhibits improved grip strength as compared to a serum CK level prior to contacting.

148. The method of claim 133, wherein the correction is permanent skipping of said mutant DMD exon.

149. The method of claim 133, wherein the correction is permanent skipping of more than one mutant DMD exon.

150. The method of claim 133, wherein the Cpf1 or Cas9 and/or DMD guide RNA are delivered to a human iPSC with an adeno-associated viral vector.
